SEQUENCE LISTING (87 sequences)

SEQ ID NO: 1 - DNA, 1176 bp, Saccharomyces cerevisiae
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

SEQ ID NO: 2 - protein, 391 aa, Saccharomyces cerevisiae
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

SEQ ID NO: 3 - DNA, 1323 bp, Saccharomyces cerevisiae
atgcttgctgtcagaagattaacaagatacacattccttaagcgaacgcatccggtgttatatactcgtcgtgcatataaaattttgccttcaagatctactttcctaagaagatcattattacaaacacaactgcactcaaagatgactgctcatactaatatcaaacagcacaaacactgtcatgaggaccatcctatcagaagatcggactctgccgtgtcaattgtacatttgaaacgtgcgcccttcaaggttacagtgattggttctggtaactgggggaccaccatcgccaaagtcattgcggaaaacacagaattgcattcccatatcttcgagccagaggtgagaatgtgggtttttgatgaaaagatcggcgacgaaaatctgacggatatcataaatacaagacaccagaacgttaaatatctacccaatattgacctgccccataatctagtggccgatcctgatcttttacactccatcaagggtgctgacatccttgttttcaacatccctcatcaatttttaccaaacatagtcaaacaattgcaaggccacgtggcccctcatgtaagggccatctcgtgtctaaaagggttcgagttgggctccaagggtgtgcaattgctatcctcctatgttactgatgagttaggaatccaatgtggcgcactatctggtgcaaacttggcaccggaagtggccaaggagcattggtccgaaaccaccgtggcttaccaactaccaaaggattatcaaggtgatggcaaggatgtagatcataagattttgaaattgctgttccacagaccttacttccacgtcaatgtcatcgatgatgttgctggtatatccattgccggtgccttgaagaacgtcgtggcacttgcatgtggtttcgtagaaggtatgggatggggtaacaatgcctccgcagccattcaaaggctgggtttaggtgaaattatcaagttcggtagaatgtttttcccagaatccaaagtcgagacctactatcaagaatccgctggtgttgcagatctgatcaccacctgctcaggcggtagaaacgtcaaggttgccacatacatggccaagaccggtaagtcagccttggaagcagaaaaggaattgcttaacggtcaatccgcccaagggataatcacatgcagagaagttcacgagtggctacaaacatgtgagttgacccaagaattcccattattcgaggcagtctaccagatagtctacaacaacgtccgcatggaagacctaccggagatgattgaagagctagacatcgatgacgaatag

SEQ ID NO: 4 - protein, 440 aa, Saccharomyces cerevisiae
MLAVRRLTRYTFLKRTHPVLYTRRAYKILPSRSTFLRRSLLQTQLHSKMTAHTNIKQHKHCHEDHPIRRSDSAVSIVHLKRAPFKVTVIGSGNWGTTIAKVIAENTELHSHIFEPEVRMWVFDEKIGDENLTDIINTRHQNVKYLPNIDLPHNLVADPDLLHSIKGADILVFNIPHQFLPNIVKQLQGHVAPHVRAISCLKGFELGSKGVQLLSSYVTDELGIQCGALSGANLAPEVAKEHWSETTVAYQLPKDYQGDGKDVDHKILKLLFHRPYFHVNVIDDVAGISIAGALKNVVALACGFVEGMGWGNNASAAIQRLGLGEIIKFGRMFFPESKVETYYQESAGVADLITTCSGGRNVKVATYMAKTGKSALEAEKELLNGQSAQGIITCREVHEWLQTCELTQEFPLFEAVYQIVYNNVRMEDLPEMIEELDIDDE

SEQ ID NO: 5 - DNA, 816 bp, Saccharomyces cerevisiae
atgaaacgtttcaatgttttaaaatatatcagaacaacaaaagcaaatatacaaaccatcgcaatgcctttgaccacaaaacctttatctttgaaaatcaacgccgctctattcgatgttgacggtaccatcatcatctctcaaccagccattgctgctttctggagagatttcggtaaagacaagccttacttcgatgccgaacacgttattcacatctctcacggttggagaacttacgatgccattgccaagttcgctccagactttgctgatgaagaatacgttaacaagctagaaggtgaaatcccagaaaagtacggtgaacactccatcgaagttccaggtgctgtcaagttgtgtaatgctttgaacgccttgccaaaggaaaaatgggctgtcgccacctctggtacccgtgacatggccaagaaatggttcgacattttgaagatcaagagaccagaatacttcatcaccgccaatgatgtcaagcaaggtaagcctcacccagaaccatacttaaagggtagaaacggtttgggtttcccaattaatgaacaagacccatccaaatctaaggttgttgtctttgaagacgcaccagctggtattgctgctggtaaggctgctggctgtaaaatcgttggtattgctaccactttcgatttggacttcttgaaggaaaagggttgtgacatcattgtcaagaaccacgaatctatcagagtcggtgaatacaacgctgaaaccgatgaagtcgaattgatctttgatgactacttatacgctaaggatgacttgttgaaatggtaa

SEQ ID NO: 6 - protein, 271 aa, Saccharomyces cerevisiae
MKRFNVLKYIRTTKANIQTIAMPLTTKPLSLKINAALFDVDGTIIISQPAIAAFWRDFGKDKPYFDAEHVIHISHGWRTYDAIAKFAPDFADEEYVNKLEGEIPEKYGEHSIEVPGAVKLCNALNALPKEKWAVATSGTRDMAKKWFDILKIKRPEYFITANDVKQGKPHPEPYLKGRNGLGFPINEQDPSKSKVVVFEDAPAGIAAGKAAGCKIVGIATTFDLDFLKEKGCDIIVKNHESIRVGEYNAETDEVELIFDDYLYAKDDLLKW

SEQ ID NO: 7 - DNA, 753 bp, Saccharomyces cerevisiae
atgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaa

SEQ ID NO: 8 - protein, 250 aa, Saccharomyces cerevisiae
MGLTTKPLSLKVNAALFDVDGTIIISQPAIAAFWRDFGKDKPYFDAEHVIQVSHGWRTFDAIAKFAPDFANEEYVNKLEAEIPVKYGEKSIEVPGAVKLCNALNALPKEKWAVATSGTRDMAQKWFEHLGIRRPKYFITANDVKQGKPHPEPYLKGRNGLGYPINEQDPSKSKVVVFEDAPAGIAAGKAAGCKIIGIATTFDLDFLKEKGCDIIVKNHESIRVGGYNAETDEVEFIFDDYLYAKDDLLKW

SEQ ID NO: 9 - DNA, 1668 bp, Klebsiella pneumoniae
  CDS (1)..(1668)
atgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgaactggacggcaaacgccgggaccagtttgacatgatcgaccgatttatcgccgattacgcgatcaacgttgagcgcacagagcaggcaatgcgcctggaggcggtggaaatagcccgtatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctacgcgccgtttaacgccctggcgctgttggtcggttcgcagtgcggccgccccggcgtgttgacgcagtgctcggtggaagaggccaccgagctggagctgggcatgcgtggcttaaccagctacgccgagacggtgtcggtctacggcaccgaagcggtatttaccgacggcgatgatacgccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattactaaaggcgccggggttcagggactgcaaaacggcgcggtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacatcctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcggcagttcgaggtggtgagtgcggtcaacgacatcaatgactatcaggggccgggcaccggctatcgcatctctgccgaacgctgggcggagatcaaaaatattccgggcgtggttcagcccgacaccattgaataa

SEQ ID NO: 10 - protein, 555 aa, Klebsiella pneumoniae
MKRSKRFAVLAQRPVNQDGLIGEWPEEGLIAMDSPFDPVSSVKVDNGLIVELDGKRRDQFDMIDRFIADYAINVERTEQAMRLEAVEIARMLVDIHVSREEIIAITTAITPAKAVEVMAQMNVVEMMMALQKMRARRTPSNQCHVTNLKDNPVQIAADAAEAGIRGFSEQETTVGIARYAPFNALALLVGSQCGRPGVLTQCSVEEATELELGMRGLTSYAETVSVYGTEAVFTDGDDTPWSKAFLASAYASRGLKMRYTSGTGSEALMGYSESKSMLYLESRCIFITKGAGVQGLQNGAVSCIGMTGAVPSGIRAVLAENLIASMLDLEVASANDQTFSHSDIRRTARTLMQMLPGTDFIFSGYSAVPNYDNMFAGSNFDAEDFDDYNILQRDLMVDGGLRPVTEAETIAIRQKAARAIQAVFRELGLPPIADEEVEAATYAHGSNEMPPRNVVEDLSAVEEMMKRNITGLDIVGALSRSGFEDIASNILNMLRQRVTGDYLQTSAILDRQFEVVSAVNDINDYQGPGTGYRISAERWAEIKNIPGVVQPDTIE

SEQ ID NO: 11 - DNA, 585 bp, Klebsiella pneumoniae
  CDS (1)..(585)
gtgcaacagacaacccaaattcagccctcttttaccctgaaaacccgcgagggcggggtagcttctgccgatgaacgcgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgctgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacatcgacttagtaagggagtga

SEQ ID NO: 12 - protein, 194 aa, Klebsiella pneumoniae
VQQTTQIQPSFTLKTREGGVASADERADEVVIGVGPAFDKHQHHTLIDMPHGAILKELIAGVEEEGLHARVVRILRTSDVSFMAWDAANLSGSGIGIGIQSKGTTVIHQRDLLPLSNLELFSQAPLLTLETYRQIGKNAARYARKESPSPVPVVNDQMVRPKFMAKAALFHIKETKHVVQDAEPVTLHIDLVRE

SEQ ID NO: 13 - DNA, 426 bp, Klebsiella pneumoniae
  CDS (1)..(426)
atgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaa

SEQ ID NO: 14 - protein, 141 aa, Klebsiella pneumoniae
MSEKTMRVQDYPLATRCPEHILTPTGKPLTDITLEKVLSGEVGPQDVRISRQTLEYQAQIAEQMQRHAVARNFRRAAELIAIPDERILAIYNALRPFRSSQAELLAIADELEHTWHATVNAAFVRESAEVYQQRHKLRKGS

SEQ ID NO: 15 - DNA, 1539 bp, Escherichia coli
  CDS (1)..(1539)
atgaccaataatcccccttcagcacagattaagcccggcgagtatggtttccccctcaagttaaaagcccgctatgacaactttattggcggcgaatgggtagcccctgccgacggcgagtattaccagaatctgacgccggtgaccgggcagctgctgtgcgaagtggcgtcttcgggcaaacgagacatcgatctggcgctggatgctgcgcacaaagtgaaagataaatgggcgcacacctcggtgcaggatcgtgcggcgattctgtttaagattgccgatcgaatggaacaaaacctcgagctgttagcgacagctgaaacctgggataacggcaaacccattcgcgaaaccagtgctgcggatgtaccgctggcgattgaccatttccgctatttcgcctcgtgtattcgggcgcaggaaggtgggatcagtgaagttgatagcgaaaccgtggcctatcatttccatgaaccgttaggcgtggtggggcagattatcccgtggaacttcccgctgctgatggcgagctggaaaatggctcccgcgctggcggcgggcaactgtgtggtgctgaaacccgcacgtcttaccccgctttctgtactgctgctaatggaaattgtcggtgatttactgccgccgggcgtggtgaacgtggtcaatggcgcaggtggggtaattggcgaatatctggcgacctcgaaacgcatcgccaaagtggcgtttaccggctcaacggaagtgggccaacaaattatgcaatacgcaacgcaaaacattattccggtgacgctggagttgggcggtaagtcgccaaatatcttctttgctgatgtgatggatgaagaagatgcctttttcgataaagcgctggaaggctttgcactgtttgcctttaaccagggcgaagtttgcacctgtccgagtcgtgctttagtgcaggaatctatctacgaacgctttatggaacgcgccatccgccgtgtcgaaagcattcgtagcggtaacccgctcgacagcgtgacgcaaatgggcgcgcaggtttctcacgggcaactggaaaccatcctcaactacattgatatcggtaaaaaagagggcgctgacgtgctcacaggcgggcggcgcaagctgctggaaggtgaactgaaagacggctactacctcgaaccgacgattctgtttggtcagaacaatatgcgggtgttccaggaggagatttttggcccggtgctggcggtgaccaccttcaaaacgatggaagaagcgctggagctggcgaacgatacgcaatatggcctgggcgcgggcgtctggagccgcaacggtaatctggcctataagatggggcgcggcatacaggctgggcgcgtgtggaccaactgttatcacgcttacccggcacatgcggcgtttggtggctacaaacaatcaggtatcggtcgcgaaacccacaagatgatgctggagcattaccagcaaaccaagtgcctgctggtgagctactcggataaaccgttggggctgttctga

SEQ ID NO: 16 - protein, 512 aa, Escherichia coli
MTNNPPSAQIKPGEYGFPLKLKARYDNFIGGEWVAPADGEYYQNLTPVTGQLLCEVASSGKRDIDLALDAAHKVKDKWAHTSVQDRAAILFKIADRMEQNLELLATAETWDNGKPIRETSAADVPLAIDHFRYFASCIRAQEGGISEVDSETVAYHFHEPLGVVGQIIPWNFPLLMASWKMAPALAAGNCVVLKPARLTPLSVLLLMEIVGDLLPPGVVNVVNGAGGVIGEYLATSKRIAKVAFTGSTEVGQQIMQYATQNIIPVTLELGGKSPNIFFADVMDEEDAFFDKALEGFALFAFNQGEVCTCPSRALVQESIYERFMERAIRRVESIRSGNPLDSVTQMGAQVSHGQLETILNYIDIGKKEGADVLTGGRRKLLEGELKDGYYLEPTILFGQNNMRVFQEEIFGPVLAVTTFKTMEEALELANDTQYGLGAGVWSRNGNLAYKMGRGIQAGRVWTNCYHAYPAHAAFGGYKQSGIGRETHKMMLEHYQQTKCLLVSYSDKPLGLF

SEQ ID NO: 17 - DNA, 1440 bp, Escherichia coli
  CDS (1)..(1440)
atgtcagtacccgttcaacatcctatgtatatcgatggacagtttgttacctggcgtggagacgcatggattgatgtggtaaaccctgctacagaggctgtcatttcccgcatacccgatggtcaggccgaggatgcccgtaaggcaatcgatgcagcagaacgtgcacaaccagaatgggaagcgttgcctgctattgaacgcgccagttggttgcgcaaaatctccgccgggatccgcgaacgcgccagtgaaatcagtgcgctgattgttgaagaagggggcaagatccagcagctggctgaagtcgaagtggcttttactgccgactatatcgattacatggcggagtgggcacggcgttacgagggcgagattattcaaagcgatcgtccaggagaaaatattcttttgtttaaacgtgcgcttggtgtgactaccggcattctgccgtggaacttcccgttcttcctcattgcccgcaaaatggctcccgctcttttgaccggtaataccatcgtcattaaacctagtgaatttacgccaaacaatgcgattgcattcgccaaaatcgtcgatgaaataggccttccgcgcggcgtgtttaaccttgtactggggcgtggtgaaaccgttgggcaagaactggcgggtaacccaaaggtcgcaatggtcagtatgacaggcagcgtctctgcaggtgagaagatcatggcgactgcggcgaaaaacatcaccaaagtgtgtctggaattggggggtaaagcaccagctatcgtaatggacgatgccgatcttgaactggcagtcaaagccatcgttgattcacgcgtcattaatagtgggcaagtgtgtaactgtgcagaacgtgtttatgtacagaaaggcatttatgatcagttcgtcaatcggctgggtgaagcgatgcaggcggttcaatttggtaaccccgctgaacgcaacgacattgcgatggggccgttgattaacgccgcggcgctggaaagggtcgagcaaaaagtggcgcgcgcagtagaagaaggggcgagagtggcgttcggtggcaaagcggtagaggggaaaggatattattatccgccgacattgctgctggatgttcgccaggaaatgtcgattatgcatgaggaaacctttggcccggtgctgccagttgtcgcatttgacacgctggaagatgctatctcaatggctaatgacagtgattacggcctgacctcatcaatctatacccaaaatctgaacgtcgcgatgaaagccattaaagggctgaagtttggtgaaacttacatcaaccgtgaaaacttcgaagctatgcaaggcttccacgccggatggcgtaaatccggtattggcggcgcagatggtaaacatggcttgcatgaatatctgcagacccaggtggtttatttacagtcttaa

SEQ ID NO: 18 - protein, 479 aa, Escherichia coli
MSVPVQHPMYIDGQFVTWRGDAWIDVVNPATEAVISRIPDGQAEDARKAIDAAERAQPEWEALPAIERASWLRKISAGIRERASEISALIVEEGGKIQQLAEVEVAFTADYIDYMAEWARRYEGEIIQSDRPGENILLFKRALGVTTGILPWNFPFFLIARKMAPALLTGNTIVIKPSEFTPNNAIAFAKIVDEIGLPRGVFNLVLGRGETVGQELAGNPKVAMVSMTGSVSAGEKIMATAAKNITKVCLELGGKAPAIVMDDADLELAVKAIVDSRVINSGQVCNCAERVYVQKGIYDQFVNRLGEAMQAVQFGNPAERNDIAMGPLINAAALERVEQKVARAVEEGARVAFGGKAVEGKGYYYPPTLLLDVRQEMSIMHEETFGPVLPVVAFDTLEDAISMANDSDYGLTSSIYTQNLNVAMKAIKGLKFGETYINRENFEAMQGFHAGWRKSGIGGADGKHGLHEYLQTQVVYLQS

SEQ ID NO: 19 - DNA, 1488 bp, Escherichia coli
  CDS (1)..(1488)
atgaattttcatcatctggcttactggcaggataaagcgttaagtctcgccattgaaaaccgcttatttattaacggtgaatatactgctgcggcggaaaatgaaacctttgaaaccgttgatccggtcacccaggcaccgctggcgaaaattgcccgcggcaagagcgtcgatatcgaccgtgcgatgagcgcagcacgcggcgtatttgaacgcggcgactggtcactctcttctccggctaaacgtaaagcggtactgaataaactcgccgatttaatggaagcccacgccgaagagctggcactgctggaaactctcgacaccggcaaaccgattcgtcacagtctgcgtgatgatattcccggcgcggcgcgcgccattcgctggtacgccgaagcgatcgacaaagtgtatggcgaagtggcgaccaccagtagccatgagctggcgatgatcgtgcgtgaaccggtcggcgtgattgccgccatcgtgccgtggaacttcccgctgttgctgacttgctggaaactcggcccggcgctggcggcgggaaacagcgtgattctaaaaccgtctgaaaaatcaccgctcagtgcgattcgtctcgcggggctggcgaaagaagcaggcttgccggatggtgtgttgaacgtggtgacgggttttggtcatgaagccgggcaggcgctgtcgcgtcataacgatatcgacgccattgcctttaccggttcaacccgtaccgggaaacagctgctgaaagatgcgggcgacagcaacatgaaacgcgtctggctggaagcgggcggcaaaagcgccaacatcgttttcgctgactgcccggatttgcaacaggcggcaagcgccaccgcagcaggcattttctacaaccagggacaggtgtgcatcgccggaacgcgcctgttgctggaagagagcatcgccgatgaattcttagccctgttaaaacagcaggcgcaaaactggcagccgggccatccacttgatcccgcaaccaccatgggcaccttaatcgactgcgcccacgccgactcggtccatagctttattcgggaaggcgaaagcaaagggcaactgttgttggatggccgtaacgccgggctggctgccgccatcggcccgaccatctttgtggatgtggacccgaatgcgtccttaagtcgcgaagagattttcggtccggtgctggtggtcacgcgtttcacatcagaagaacaggcgctacagcttgccaacgacagccagtacggccttggcgcggcggtatggacgcgcgacctctcccgcgcgcaccgcatgagccgacgcctgaaagccggttccgtcttcgtcaataactacaacgacggcgatatgaccgtgccgtttggcggctataagcagagcggcaacggtcgcgacaaatccctgcatgcccttgaaaaattcactgaactgaaaaccatctggataagcctggaggcctga

SEQ ID NO: 20 - protein, 495 aa, Escherichia coli
MNFHHLAYWQDKALSLAIENRLFINGEYTAAAENETFETVDPVTQAPLAKIARGKSVDIDRAMSAARGVFERGDWSLSSPAKRKAVLNKLADLMEAHAEELALLETLDTGKPIRHSLRDDIPGAARAIRWYAEAIDKVYGEVATTSSHELAMIVREPVGVIAAIVPWNFPLLLTCWKLGPALAAGNSVILKPSEKSPLSAIRLAGLAKEAGLPDGVLNVVTGFGHEAGQALSRHNDIDAIAFTGSTRTGKQLLKDAGDSNMKRVWLEAGGKSANIVFADCPDLQQAASATAAGIFYNQGQVCIAGTRLLLEESIADEFLALLKQQAQNWQPGHPLDPATTMGTLIDCAHADSVHSFIREGESKGQLLLDGRNAGLAAAIGPTIFVDVDPNASLSREEIFGPVLVVTRFTSEEQALQLANDSQYGLGAAVWTRDLSRAHRMSRRLKAGSVFVNNYNDGDMTVPFGGYKQSGNGRDKSLHALEKFTELKTIWISLEA

SEQ ID NO: 21 - DNA, 1395 bp, Escherichia coli
atgcctgacgctaaaaaacaggggcggtcaaacaaggcaatgacgtttttcgtctgcttccttgccgctctggcgggattactctttggcctggatatcggtgtaattgctggcgcactgccgtttattgcagatgaattccagattacttcgcacacgcaagaatgggtcgtaagctccatgatgttcggtgcggcagtcggtgcggtgggcagcggctggctctcctttaaactcgggcgcaaaaagagcctgatgatcggcgcaattttgtttgttgccggttcgctgttctctgcggctgcgccaaacgttgaagtactgattctttcccgcgttctactggggctggcggtgggtgtggcctcttataccgcaccgctgtacctctctgaaattgcgccggaaaaaattcgtggcagtatgatctcgatgtatcagttgatgatcactatcgggatcctcggtgcttatctttctgataccgccttcagctacaccggtgcatggcgctggatgctgggtgtgattatcatcccggcaattttgctgctgattggtgtcttcttcctgccagacagcccacgttggtttgccgccaaacgccgttttgttgatgccgaacgcgtgctgctacgcctgcgtgacaccagcgcggaagcgaaacgcgaactggatgaaatccgtgaaagtttgcaggttaaacagagtggctgggcgctgtttaaagagaacagcaacttccgccgcgcggtgttccttggcgtactgttgcaggtaatgcagcaattcaccgggatgaacgtcatcatgtattacgcgccgaaaatcttcgaactggcgggttataccaacactaccgagcaaatgtgggggaccgtgattgtcggcctgaccaacgtacttgccacctttatcgcaatcggccttgttgaccgctggggacgtaaaccaacgctaacgctgggcttcctggtgatggctgctggcatgggcgtactcggtacaatgatgcatatcggtattcactctccgtcggcgcagtatttcgccatcgccatgctgctgatgtttattgtcggttttgccatgagtgccggtccgctgatttgggtactgtgctccgaaattcagccgctgaaaggccgcgattttggcatcacctgctccactgccaccaactggattgccaacatgatcgttggcgcaacgttcctgaccatgctcaacacgctgggtaacgccaacaccttctgggtgtatgcggctctgaacgtactgtttatcctgctgacattgtggctggtaccggaaaccaaacacgtttcgctggaacatattgaacgtaatctgatgaaaggtcgtaaactgcgcgaaataggcgctcacgattaa

SEQ ID NO: 22 - protein, 464 aa, Escherichia coli
MPDAKKQGRSNKAMTFFVCFLAALAGLLFGLDIGVIAGALPFIADEFQITSHTQEWVVSSMMFGAAVGAVGSGWLSFKLGRKKSLMIGAILFVAGSLFSAAAPNVEVLILSRVLLGLAVGVASYTAPLYLSEIAPEKIRGSMISMYQLMITIGILGAYLSDTAFSYTGAWRWMLGVIIIPAILLLIGVFFLPDSPRWFAAKRRFVDAERVLLRLRDTSAEAKRELDEIRESLQVKQSGWALFKENSNFRRAVFLGVLLQVMQQFTGMNVIMYYAPKIFELAGYTNTTEQMWGTVIVGLTNVLATFIAIGLVDRWGRKPTLTLGFLVMAAGMGVLGTMMHIGIHSPSAQYFAIAMLLMFIVGFAMSAGPLIWVLCSEIQPLKGRDFGITCSTATNWIANMIVGATFLTMLNTLGNANTFWVYAALNVLFILLTLWLVPETKHVSLEHIERNLMKGRKLREIGAHD

SEQ ID NO: 23 - DNA, 1248 bp, Escherichia coli
atggcactgaatattccattcagaaatgcgtactatcgttttgcatccagttactcatttctcttttttatttcctggtcgctgtggtggtcgttatacgctatttggctgaaaggacatctaggattaacagggacggaattaggtacactttattcggtcaaccagtttaccagcattctatttatgatgttctacggcatcgttcaggataaactcggtctgaagaaaccgctcatctggtgtatgagtttcattctggtcttgaccggaccgtttatgatttacgtttatgaaccgttactgcaaagcaatttttctgtaggtctaattctgggggcgctcttttttggcctggggtatctggcgggatgcggtttgcttgacagcttcaccgaaaaaatggcgcgaaattttcatttcgaatatggaacagcgcgcgcctggggatcttttggctatgctattggcgcgttctttgccggtatattttttagtatcagtccccatatcaacttctggttggtctcgctatttggcgctgtatttatgatgatcaacatgcgttttaaagataaggatcaccagtgcatagcggcggatgcgggaggggtaaaaaaagaggattttatcgcagttttcaaggatcgaaacttctgggttttcgtcatatttattgtggggacgtggtctttctataacatttttgatcaacaactctttcctgtcttttatgcaggtttattcgaatcacacgatgtaggaacgcgcctgtatggttatctcaactcattccaggtggtactcgaagcgctgtgcatggcgattattcctttctttgtgaatcgggtagggccaaaaaatgcattacttatcggtgttgtgattatggcgttgcgtatcctttcctgcgcgttgttcgttaacccctggattatttcattagtgaagctgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcctgagtaaaaaacgcgagcaaatagttatggaaacgcctgtaccttcagcaatatag

SEQ ID NO: 24 - protein, 415 aa, Escherichia coli
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCIAADAGGVKKEDFIAVFKDRNFWVFVIFIVGTWSFYNIFDQQLFPVFYAGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 25 - DNA, 1248 bp, Escherichia coli
atggcactgaatattccattcagaaatgcgtactatcgttttgcatccagttactcatttctcttttttatttcctggtcgctgtggtggtcgttatacgctatttggctgaaaggacatctagggttgacagggacggaattaggtacactttattcggtcaaccagtttaccagcattctatttatgatgttctacggcatcgttcaggataaactcggtctgaagaaaccgctcatctggtgtatgagtttcatcctggtcttgaccggaccgtttatgatttacgtttatgaaccgttactgcaaagcaatttttctgtaggtctaattctgggggcgctattttttggcttggggtatctggcgggatgcggtttgcttgatagcttcaccgaaaaaatggcgcgaaattttcatttcgaatatggaacagcgcgcgcctggggatcttttggctatgctattggcgcgttctttgccggcatattttttagtatcagtccccatatcaacttctggttggtctcgctatttggcgctgtatttatgatgatcaacatgcgttttaaagataaggatcaccagtgcgtagcggcagatgcgggaggggtaaaaaaagaggattttatcgcagttttcaaggatcgaaacttctgggttttcgtcatatttattgtggggacgtggtctttctataacatttttgatcaacaactttttcctgtcttttattcaggtttattcgaatcacacgatgtaggaacgcgcctgtatggttatctcaactcattccaggtggtactcgaagcgctgtgcatggcgattattcctttctttgtgaatcgggtagggccaaaaaatgcattacttatcggagttgtgattatggcgttgcgtatcctttcctgcgcgctgttcgttaacccctggattatttcattagtgaagttgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcttgagtaaaaaacgcgagcaaatagttatggaaacgcctgtaccttcagcaatatag

SEQ ID NO: 26 - protein, 415 aa, Escherichia coli
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 27 - DNA, 1326 bp, Bifidobacterium lactis
atggcaacaaccacgaaggtgtggaggaacccctcctacctgcaaagctcaaccggcatcttcctgttcttctgctcctggggcatctggtggtcgttcttccagcgctggctcaactcgatgggactcaacggcgcgaaagtgggcacgatctattcgatcaactcgctggccacgctcatcctcatgttcgggtacggcctcatccaggacaatctcggactcaagcgccgtcttgtgctcgtcatctcggcgatcgccgcactcgtcggacccttcgtgcagttcgtgtacgcgccgctgatgaggacgaacatgatggccgccgcactcgtgggctccgtcgttctctccgcgggcttcatggcaggctgctcgctcatagagcccgtgaccgaacggtacagccgccgtttcaacttagagtacggccaatcccgcgcatggggttccttcggatatgccattgtggcgcttgtcgccggcttcgtgttcaacatcaacccgatgatcaacttctggctcggctccgcattcggcgtgggcatgctcatcgtgtacctcacctggtatccggccgagcagcgcgaagcgctcaaggaagccgccgatccgaatgccgcgccaactaacccgaccatcaaagacatgctcggcgtgctcaagatgcccacgctgtgggtgctcatcgtgttcatgctgctcaccaacacgttctacaccgtattcgaccagcagatgttccccacctactacgcctcgctcttcccgaatgaggccaccggcaacgccgtctacggcacgctcaactcggtgcaggtgttctgcgaatccgcgatgatgggcgtcgtgccgatcatcatgcgcaaggtaggtgtgcgcaacgcgttgctgctcggatccacggtgatgttccttcgcatcgggctgtgcggcatcttccacgatccggtgtccatctcgatcgtcaaaatgttccacgccattgaagttccgctgttctgcctgccggcgttccgctacttcacgctccacttcaatccgaagctctccgcgacgctctacatggtcggcttccagattgcctcacagatcggccaggtcgtcttctccaccccgctcggcatgctgcatgaccgcatgggcgaccgcacgacgttcctgacgatctccgccatcgtgcttgctgccaccgtctacggattcttcgtgatcaagcgcgacgacgagcaggtggatggcgatccgttcatccgcgattcgaagaagctgccgtcgctcgccaccgacgaggcgatcctctccgcggattccgaggatatgtaa

SEQ ID NO: 28 - protein, 441 aa, Bifidobacterium lactis
MATTTKVWRNPSYLQSSTGIFLFFCSWGIWWSFFQRWLNSMGLNGAKVGTIYSINSLATLILMFGYGLIQDNLGLKRRLVLVISAIAALVGPFVQFVYAPLMRTNMMAAALVGSVVLSAGFMAGCSLIEPVTERYSRRFNLEYGQSRAWGSFGYAIVALVAGFVFNINPMINFWLGSAFGVGMLIVYLTWYPAEQREALKEAADPNAAPTNPTIKDMLGVLKMPTLWVLIVFMLLTNTFYTVFDQQMFPTYYASLFPNEATGNAVYGTLNSVQVFCESAMMGVVPIIMRKVGVRNALLLGSTVMFLRIGLCGIFHDPVSISIVKMFHAIEVPLFCLPAFRYFTLHFNPKLSATLYMVGFQIASQIGQVVFSTPLGMLHDRMGDRTTFLTISAIVLAATVYGFFVIKRDDEQVDGDPFIRDSKKLPSLATDEAILSADSEDM

SEQ ID NO: 29 - DNA, 858 bp, Streptococcus pneumoniae
ttattgatgactgtccccggtttagttttaacctttatctttaaatacatccctatgtatggggttttaatcgcatttaaagattacaatcctttaaaaggaattttagggagtgattggattggtttttctgagtttacaaaattcatatcctctcccaactttggtatcttgttagccaacacattaaaattaagtatctatggtttattgcttggctttttaccaccaatcattctcgcgattatgctcaatcaactcttgagtgaaaaagtcaaaaaacgaattcagctcattttatacgcaccaaactttatctcagtcgttgttattgtcggtatgattttcctcttcttttcagtgggaggaccaatcaacaattttctttctatgtttggaatgaaggctgacttcttgacaaatccagacttctttagacctttatacatctttagtggtatctggcaaggaatgggctgggcttcaacgctctacacggcaacattggtaaatgtagatccagccttagtagaagcagcccgactggatggagccaatatcttccaacgaatctggcacattgatattccagctcttaagcctattatggttatccaatttgttttagctgcaggtggaattatgaatgtcggatatgaaaaagcattcttgatgcagacatcgttaaatttgccaacttctgaaattatctcgacatatgtctataaagttggtcttgtatcaggagactattcttactcaacagcggttggtttgtttaatgcagtgattaacgtagtattgcttgttgcagttaaccaaatcgttaaacgcatgaataatggtgaaggaatttaa

SEQ ID NO: 30 - protein, 305 aa, Streptococcus pneumoniae
MNSKAKQVSLWERIKKQKLLLLMTVPGLVLTFIFKYIPMYGVLIAFKDYNPLKGILGSDWIGFSEFTKFISSPNFGILLANTLKLSIYGLLLGFLPPIILAIMLNQLLSEKVKKRIQLILYAPNFISVVVIVGMIFLFFSVGGPINNFLSMFGMKADFLTNPDFFRPLYIFSGIWQGMGWASTLYTATLVNVDPALVEAARLDGANIFQRIWHIDIPALKPIMVIQFVLAAGGIMNVGYEKAFLMQTSLNLPTSEIISTYVYKVGLVSGDYSYSTAVGLFNAVINVVLLVAVNQIVKRMNNGEGI

SEQ ID NO: 31 - DNA, 918 bp, Streptococcus pneumoniae
atggtgaaggaatttaaggaggaaagtatgaaaaattcgattatggatacaaaatttgatagacgtatcttactcttaaataaaatcattattgtctttatcgttttgatgactttgcttcctttactttatatcgtcgtagcatcctttatggatcctaaggttctggttagtagagggattagctttaatccagccgattggactgtagaaggttaccagcgtgtattcagtgaccaatctattctaagaggttttatcaattctctactatactcttttggatttgcagctttaacagtcttgctatctgtgtttacagcttatcctctttctaagaaagacttggttggacgtcgttggattaactacttcttgattgtaactatgttctttggtggtggtttagtcccaacttacttgctcgtaaaagaattgggaatgctcaatactccatgggctatcattgttccaggtgctgttaacgtttggaatattattcttgctagggcctatttccaaggattgcctgaagaattagttgaagctgctgtcattgatggtgcaaatgatttacagattttcttcaaaatcatgcttcctcttgcaaaaccaattatgtttgttctcttcctttatgcttttgtaggacagtggaactcatactttgatgcaatgatttatatcaaggatccaaacttggaaccattgcaacttgtacttcgtaaaattctcattcagagccaaccaggtcaagacatgattggagcacaagcggctatgaatgaaatgaaacgtttagctgaattgattaaatacgcaactattgtcatttccagcttgccattgattgttatgtatccattcttccaaaaatactttgataaaggaattatggctggttcacttaaaggataa

SEQ ID NO: 32 - protein, 305 aa, Streptococcus pneumoniae
MVKEFKEESMKNSIMDTKFDRRILLLNKIIIVFIVLMTLLPLLYIVVASFMDPKVLVSRGISFNPADWTVEGYQRVFSDQSILRGFINSLLYSFGFAALTVLLSVFTAYPLSKKDLVGRRWINYFLIVTMFFGGGLVPTYLLVKELGMLNTPWAIIVPGAVNVWNIILARAYFQGLPEELVEAAVIDGANDLQIFFKIMLPLAKPIMFVLFLYAFVGQWNSYFDAMIYIKDPNLEPLQLVLRKILIQSQPGQDMIGAQAAMNEMKRLAELIKYATIVISSLPLIVMYPFFQKYFDKGIMAGSLKG

SEQ ID NO: 33 - DNA, 1617 bp, Streptococcus pneumoniae
atgaaattcaaaacattctcaaaatcagcagttttgttgacagctagtttagcagtacttgcagcctgtggctcaaaaaatacagcttcaagtccagattataagttggaaggtgtaacattcccgcttcaagaaaagaaaacattgaagtttatgacagccagttcaccgttatctcctaaagacccaaatgaaaagttaattttgcaacgtttggagaaggaaactggcgttcatattgactggaccaactaccaatccgactttgcagaaaaacgtaacttggatatttctagtggtgatttaccagatgctatccacaacgacggagcttcagatgtggacttgatgaactgggctaaaaaaggtgttattattccagttgaagatttgattgataaatacatgccaaatcttaagaaaattttggatgagaaaccagagtacaaggccttgatgacagcacctgatgggcacatttactcatttccatggattgaagagcttggagatggtaaagagtctattcacagtgtcaacgatatggcttggattaacaaagattggcttaagaaacttggtcttgaaatgccaaaaactactgatgatttgattaaagtcctagaagctttcaaaaacggggatccaaatggaaatggagaggctgatgaaattccattttcatttattagtggtaacggaaacgaagattttaaattcctatttgctgcatttggtataggggataacgatgatcatttagtagtaggaaatgatggcaaagttgacttcacagcagataacgataactataaagaaggtgtcaaatttatccgtcaattgcaagaaaaaggcctgattgataaagaagctttcgaacatgattggaatagttacattgctaaaggtcatgatcagaaatttggtgtttactttacatgggataagaataatgttactggaagtaacgaaagttatgatgttttaccagtacttgctggaccaagtggtcaaaaacacgtagctcgtacaaacggtatgggatttgcacgtgacaagatggttattaccagtgtaaacaaaaacctagaattgacagctaaatggattgatgcacaatacgctccactccaatctgtgcaaaataactggggaacttacggagatgacaaacaacaaaacatctttgaattggatcaagcgtcaaatagtctaaaacacttaccactaaacggaactgcaccagcagaacttcgtcaaaagactgaagtaggaggaccactagctatcctagattcatactatggtaaagtaacaaccatgcctgatgatgccaaatggcgtttggatcttatcaaagaatattatgttccttacatgagcaatgtcaataactatccaagagtctttatgacacaggaagatttggacaagattgcccatatcgaagcagatatgaatgactatatctaccgtaaacgtgctgaatggattgtaaatggcaatattgatactgagtgggatgattacaagaaagaacttgaaaaatacggactttctgattacctcgctattaaacaaaaatactacgaccaataccaagcaaacaaaaactag

SEQ ID NO: 34 - protein, 538 aa, Streptococcus pneumoniae
MKFKTFSKSAVLLTASLAVLAACGSKNTASSPDYKLEGVTFPLQEKKTLKFMTASSPLSPKDPNEKLILQRLEKETGVHIDWTNYQSDFAEKRNLDISSGDLPDAIHNDGASDVDLMNWAKKGVIIPVEDLIDKYMPNLKKILDEKPEYKALMTAPDGHIYSFPWIEELGDGKESIHSVNDMAWINKDWLKKLGLEMPKTTDDLIKVLEAFKNGDPNGNGEADEIPFSFISGNGNEDFKFLFAAFGIGDNDDHLVVGNDGKVDFTADNDNYKEGVKFIRQLQEKGLIDKEAFEHDWNSYIAKGHDQKFGVYFTWDKNNVTGSNESYDVLPVLAGPSGQKHVARTNGMGFARDKMVITSVNKNLELTAKWIDAQYAPLQSVQNNWGTYGDDKQQNIFELDQASNSLKHLPLNGTAPAELRQKTEVGGPLAILDSYYGKVTTMPDDAKWRLDLIKEYYVPYMSNVNNYPRVFMTQEDLDKIAHIEADMNDYIYRKRAEWIVNGNIDTEWDDYKKELEKYGLSDYLAIKQKYYDQYQANKN

SEQ ID NO: 35 - DNA, 1248 bp, Streptococcus mutans
atgaaaacatggcaaaaaatcgtcgttggcggtgcaggccttatgcttgcaagcagtattcttgttgcctgtggatcaaaggattcaaaatcaagttcatctgatcccaaaaccattaaactttgggttccaacaggagccaagaaatcttatcaaagtattgttcacaaatttgaaaaggattctaactataaagtaaagattattgaatctgaagacccaaaagctcaggaaaagatcaaaaaagatcctagtactgctgcagatgttttctcgctgccgcatgatcagctgggccagttagttgactctggtgttatccaagagattcctcaaaaatattcaaaagaaataaataaaaatgaaacacagcaggctgcaacaggagctatgtacaaaggtaagacttatgcttttccttttggaatcgagtctcaagtactttactataataaatcaaaactctcagctgatgatgtcacatcatatgagactattaccagcaaggcaactttcggagcaaaattcaaacaagttaatgcctatgcgactgcaccacttttctattcagtaggtgatacactctttggtaaaaatggcgaagatgccaaaggaactaactggggaaatgatgctggtgtatctgttttgaaatggattgccagtcaaaaaggtaacgctggctttgtcaatcttgacgataacaatgtcatgtctaaatttggtgatggttctgtagcttcttttgaatcaggtccttgggattatgaagccgcacaaaaggcagttggcaaaaacaacctcggtgttacggtttatccaacaataaatattaatggtcaagaagttcaacagaaagctttcttaggtgttaaactctacgctgttaatcaagctccttctaaaggaaataccaaacgtattgctgctagttataaattagcttcttacttaacaagtgctgaaagccaagaaaatcaatttaagacaaaaggacgcaacatcatcccatctaataagaccgttcaaaactctgatacagtcaaaaatcatgaactcgcacaggctgttatccaaatgggatcttcttcagattatactgttgttatgcctaaactcaaccaaatgtcaacattctggacggaaagcgcagctattcttagtgatacttacaatggtaaaattaaagaaagtgattaccttgctaaattaaaacaatttgataaagatttagcagctgctaaataa

SEQ ID NO: 36 - protein, 415 aa, Streptococcus mutans
MKTWQKIVVGGAGLMLASSILVACGSKDSKSSSSDPKTIKLWVPTGAKKSYQSIVHKFEKDSNYKVKIIESEDPKAQEKIKKDPSTAADVFSLPHDQLGQLVDSGVIQEIPQKYSKEINKNETQQAATGAMYKGKTYAFPFGIESQVLYYNKSKLSADDVTSYETITSKATFGAKFKQVNAYATAPLFYSVGDTLFGKNGEDAKGTNWGNDAGVSVLKWIASQKGNAGFVNLDDNNVMSKFGDGSVASFESGPWDYEAAQKAVGKNNLGVTVYPTININGQEVQQKAFLGVKLYAVNQAPSKGNTKRIAASYKLASYLTSAESQENQFKTKGRNIIPSNKTVQNSDTVKNHELAQAVIQMGSSSDYTVVMPKLNQMSTFWTESAAILSDTYNGKIKESDYLAKLKQFDKDLAAAK

SEQ ID NO: 37 - DNA, 1362 bp, Streptococcus mutans
atgattcagtcatcttctcatgatcagttatctgtacttgaaacttttaaaaagggcgggatagatatcaaattatcgtttgtcatcatgggatttgccaatttgatgaataagcaattcataaaaggcctcctctttctattaagtgagatagcttttctaattgcttttgtcacacaggttattccagctttttcaggcttactcactctcggtactaaaacacaagggatgcaagaaaaaattgtggatggcgttaaattacaggtggcagttgaaggcgataattcgatgctgatgctcatttttggattagcctcactaatcttttgtttggtttttgcctacatttattggtgtaatcttaaaagtgccagaaatctctatatgttaaaaaaagagggacgtcacattccatctttcaaagaagattttatgactttggcaaacggccgattccatatgactttgatgtttattcctttgattggtgttcttctttttaccattttgccactcgtttatatgatttgcctggcctttaccaattatgatcacaatcatcttccgcctaaatccctttttgattgggtagggttggctaattttggtaatgttttgaatggccgcatggctggaaccttcttccctgtcctttcttggacacttatctgggctgttttcgcaactgtgacaaactttctttttggagtcatcttggcacttattatcaatgctaagggattaaaattgaaaaaaatgtggcggactatctttgttattaccattgctgtgccgcagttcatttcacttttgctgatgagaaatttccttaatgatcaaggtccgctcaatgctttcctagaaaaaattggcctgatttctcattctctgccatttctatcagatcctacttgggcaaaattttcaattatcttcgttaatatgtgggttggtattccttttaccatgttagtcgcaacaggaattatcatgaatcttccgagtgagcaaattgaggctgcagaaattgacggcgctagtaagttccaaatttttaaatccatcactttcccgcagattcttttaattatgatgccatctttaatccagcaatttattggaaatatcaataattttaatgtcatctaccttttaaccggtggcggaccaactaattcacaattctatcaagcaggcagcacagacttattggtcacttggctttataaactaacaatgaatgctgcagactataatttagcttctgttattggtatctttatctttgccatttcagctatcttcagtcttttagcttatacgcatacagcatcatacaaggaaggagctgttaaataa

SEQ ID NO: 38 - protein, 453 aa, Streptococcus mutans
MIQSSSHDQLSVLETFKKGGIDIKLSFVIMGFANLMNKQFIKGLLFLLSEIAFLIAFVTQVIPAFSGLLTLGTKTQGMQEKIVDGVKLQVAVEGDNSMLMLIFGLASLIFCLVFAYIYWCNLKSARNLYMLKKEGRHIPSFKEDFMTLANGRFHMTLMFIPLIGVLLFTILPLVYMICLAFTNYDHNHLPPKSLFDWVGLANFGNVLNGRMAGTFFPVLSWTLIWAVFATVTNFLFGVILALIINAKGLKLKKMWRTIFVITIAVPQFISLLLMRNFLNDQGPLNAFLEKIGLISHSLPFLSDPTWAKFSIIFVNMWVGIPFTMLVATGIIMNLPSEQIEAAEIDGASKFQIFKSITFPQILLIMMPSLIQQFIGNINNFNVIYLLTGGGPTNSQFYQAGSTDLLVTWLYKLTMNAADYNLASVIGIFIFAISAIFSLLAYTHTASYKEGAVK

SEQ ID NO: 39 - DNA, 837 bp, Streptococcus mutans
atgaaaagaaaaaaacaacttcagatcggctctatctatgctttactgattctcttatccttcatttggctatttccgatcatttgggttatactgacgagttttcgcggtgaaggcacagcttatgttccttatattattccaaaaacgtggactttagataattatattaaattatttaccaattcttctttcccatttggacgctggtttttaaataccttaatcgtttcaacagccacttgtgttctgtcaacttctatcacagtggcaatggcttattcgcttagccgtattaaatttaaacaccgtaacggctttttaaaattagctcttgttctgaatatgtttccgggatttatgagtatgattgcagtttactacattctaaaagcactcaatctcacccaaacattaacatctcttgttttggtctattcttcaggagctgccttaactttctatatcgctaaaggcttttttgatacgattccttattcattggatgaatcagctatgattgatggggctacgcgtaaagatattttcttaaaaatcactctgccgctatctaagcccatcatcgtttatacggccctgttggcatttattgccccttggattgactttatttttgctcaggttattcttggagatgccaccagcaaatataccgtagcgattggactcttctctatgcttcaagctgataccattaataattggttcatggcctttgcagcaggttctgtactgatcgccattccaatcacgatactttttatcttcatgcaaaagtattacgttgaaggcattactggcggatctgttaaataa

SEQ ID NO: 40 - protein, 278 aa, Streptococcus mutans
MKRKKQLQIGSIYALLILLSFIWLFPIIWVILTSFRGEGTAYVPYIIPKTWTLDNYIKLFTNSSFPFGRWFLNTLIVSTATCVLSTSITVAMAYSLSRIKFKHRNGFLKLALVLNMFPGFMSMIAVYYILKALNLTQTLTSLVLVYSSGAALTFYIAKGFFDTIPYSLDESAMIDGATRKDIFLKITLPLSKPIIVYTALLAFIAPWIDFIFAQVILGDATSKYTVAIGLFSMLQADTINNWFMAFAAGSVLIAIPITILFIFMQKYYVEGITGGSVK

SEQ ID NO: 41 - DNA, 1134 bp, Streptococcus mutans
atgacaactttaaaacttgataacatctacaaaagatatcccaatgcaaagcattattccgttgaaaattttaatcttgacattcatgataaagaatttattgtctttgtcggtccttcaggatgcggaaagtcaaccactcttcgcatgattgctgggctggaagatattacagaaggcaacctttatattgatgataaactcatgaatgatgcctctcctaaagatcgcgatattgctatggtttttcaaaattatgctctttatcctcatatgagcgtttatgaaaatatggcttttggcctaaaacttcgtaaatacaaaaaagatgatattaataaacgtgtacacgaagctgctgaaattcttggactgacagaatttcttgaaagaaagcctgcggacctctctggcggacagcggcagcgggttgctatgggacgtgctattgtccgagatgctaaggtcttcttaatggacgaacctttgtcaaatttagatgccaaacttcgagttgccatgcgagccgaaatcgctaaaattcaccgccgcattggggcaacgactatctatgttacccatgaccaaacagaagccatgaccttagcagatcgtattgttatcatgagcgctactccaaacccagataaaaccggctctatcggtcgtattgagcagattggaacaccacaggaactctacaatgaacctgctaataaatttgttgctggcttcatcggaagccccgctatgaatttctttgaagtgaccgttgaaaaagagcgtttggttaaccaagatggtctaagccttgcgcttcctcagggacaggaaaaaattcttgaggagaaaggttatcttggtaaaaaagtcactttaggtattcgaccagaagacatctcaagtgatcaaattgtccacgagactttcccaaatgccagtgttacagctgacatactagtatcagaacttttaggcagcgaaagcatgttatatgtcaaatttggcagtactgaatttacagctcgcgtcaatgctcgtgactctcacagtcccggagaaaaagtacaattaacctttaatattgctaagggacacttctttgatttagagactgaaaaacgaatcaattaa

SEQ ID NO: 42 - protein, 377 aa, Streptococcus mutans
MTTLKLDNIYKRYPNAKHYSVENFNLDIHDKEFIVFVGPSGCGKSTTLRMIAGLEDITEGNLYIDDKLMNDASPKDRDIAMVFQNYALYPHMSVYENMAFGLKLRKYKKDDINKRVHEAAEILGLTEFLERKPADLSGGQRQRVAMGRAIVRDAKVFLMDEPLSNLDAKLRVAMRAEIAKIHRRIGATTIYVTHDQTEAMTLADRIVIMSATPNPDKTGSIGRIEQIGTPQELYNEPANKFVAGFIGSPAMNFFEVTVEKERLVNQDGLSLALPQGQEKILEEKGYLGKKVTLGIRPEDISSDQIVHETFPNASVTADILVSELLGSESMLYVKFGSTEFTARVNARDSHSPGEKVQLTFNIAKGHFFDLETEKRIN

SEQ ID NO: 43 - DNA, 927 bp, Agrobacterium tumefaciens
atgatcctgtgttgtggtgaagccctgatcgacatgctgccccggcagacgacgctgggtgaggcgggctttgccccttacgcaggcggagcggtcttcaacacggcaattgcgctggggcgtcttggcgtcccttcagccttttttaccggtctttccgacgacatgatgggcgatatcctgcgggagaccctgcgggccagcaaggtggatttcagctattgcgccaccctgtcgcgccccaccaccattgcgttcgttaagctggttgatggccatgcgacctacgctttttacgacgagaacaccgccggccggatgatcaccgaggccgaacttccggccttgggagcggattgcgaagcgctgcatttcggcgccatcagccttattcccgaaccctgcggcagcacctatgaggcgctgatgacgcgcgagcatgagacccgcgtcatctcgctcgatccgaacattcgtcccggcttcatccagaacaagcagtcgcacatggcccgcatccgccgcatggcggcgatgtctgacatcgtcaagttctcggatgaggacctggcgtggttcggtctggaaggcgacgaggacacgcttgcccgccactggctgcaccacggtgcaaaactcgtcgttgtcacccgtggcgccaagggtgccgtgggttacagcgccaatctcaaggtggaagtggcctccgagcgcgtcgaagtggtcgatacggtcggcgccggcgatacgttcgatgccggcattcttgcttcgctgaaaatgcagggcctgctgaccaaagcgcaggtggcttcgctgagcgaagagcagatcagaaaagctttggcgcttggcgcgaaagccgctgcggtcactgtctcgcgggctggcgcaaatccgcctttcgcgcatgaaatcggtttgtga

SEQ ID NO: 44 - protein, 308 aa, Agrobacterium tumefaciens
MILCCGEALIDMLPRQTTLGEAGFAPYAGGAVFNTAIALGRLGVPSAFFTGLSDDMMGDILRETLRASKVDFSYCATLSRPTTIAFVKLVDGHATYAFYDENTAGRMITEAELPALGADCEALHFGAISLIPEPCGSTYEALMTREHETRVISLDPNIRPGFIQNKQSHMARIRRMAAMSDIVKFSDEDLAWFGLEGDEDTLARHWLHHGAKLVVVTRGAKGAVGYSANLKVEVASERVEVVDTVGAGDTFDAGILASLKMQGLLTKAQVASLSEEQIRKALALGAKAAAVTVSRAGANPPFAHEIGL

SEQ ID NO: 45 - DNA, 1404 bp, Streptococcus mutans
cagctgattatgcgtcagttgaaaccctcgcttcttcaggaactgttgctgtaggtgatagcttacttgaagttaaaaaataagaaatattatcagaaagaccgtaaggtctttttgactgcttaaaagattcagtaacaatagtattaaagccttttggctaactaatacttgaaatttagcaaattatgatataatgttaagtagtccttaagggtagattaagggtattcaaatccaaaaattgatttggtaagttaagtaaaatataagaggtttattatgtctaaattatatggcagcatcgaagctggcggaacaaaatttgtctgtgctgtaggtgatgaaaattttcaaattttagaaaaagttcagttcccaacaacaacaccttatgaaacaatagaaaaaacagttgctttctttaaaaaatttgaagctgatttagccagtgttgccattggttcttttggccctattgatattgatcaaaattcagacacttatggttacattacttcaacaccaaagccaaactgggctaacgttgattttgtcggcttaatttctaaagattttaaaattccattttactttacgacagatgttaattcttctgcttatggggaaacaattgctcgttcaaatgttaaaagtctggtttattatactattggaacaggcattggagcaggggctattcaaaatggcgaattcattggcggtatgggacatacggaagctggacacgtttacatggctccgcatcccaatgatgttcatcatggttttgtaggcacctgtcctttccataaaggctgtttagaaggacttgcagcgggtcctagcttagaggctcgtactggtattcgtggtgagttaattgagcaaaactcagaagtttgggatattcaggcatactacattgctcaggcggctattcaagcgactgtcctttatcgtccgcaagtcattgtatttggcggaggcgttatggcacaagaacatatgctcaatcgggttcgtgaaaaatttacttcacttttgaatgactatcttccagttccagatgttaaagattatattgtgacaccagctgttgcagaaaatggttcagcaacattgggaaatctcgctttagctaaaaagatagcagcgcgttaattaaaaatgaattggaagattaaagcaccttctaatattcaatattaaactgttagaatttacgtgaacgaaattttcattttatgaggataatgaagtgaatataattactcttgatttcctctgaaactagatagtggtatattgaaaaacagaaaggagaacactatggaaggacctttgtttttacaatcacaaatgcataaaaaaatctggggcggcaatcggctcagaaaagaa

SEQ ID NO: 46 - protein, 293 aa, Streptococcus mutans
MSKLYGSIEAGGTKFVCAVGDENFQILEKVQFPTTTPYETIEKTVAFFKKFEADLASVAIGSFGPIDIDQNSDTYGYITSTPKPNWANVDFVGLISKDFKIPFYFTTDVNSSAYGETIARSNVKSLVYYTIGTGIGAGAIQNGEFIGGMGHTEAGHVYMAPHPNDVHHGFVGTCPFHKGCLEGLAAGPSLEARTGIRGELIEQNSEVWDIQAYYIAQAAIQATVLYRPQVIVFGGGVMAQEHMLNRVREKFTSLLNDYLPVPDVKDYIVTPAVAENGSATLGNLALAKKIAAR

SEQ ID NO: 47 - DNA, 915 bp, Escherichia coli
atgtcagccaaagtatgggttttaggggatgcggtcgtagatctcttgccagaatcagacgggcgcctactgccttgtcctggcggcgcgccagctaacgttgcggtgggaatcgccagattaggcggaacaagtgggtttataggtcgggtgggggatgatccttttggtgcgttaatgcaaagaacgctgctaactgagggagtcgatatcacgtatctgaagcaagatgaatggcaccggacatccacggtgcttgtcgatctgaacgatcaaggggaacgttcatttacgtttatggtccgccccagtgccgatctttttttagagacgacagacttgccctgctggcgacatggcgaatggttacatctctgttcaattgcgttgtctgccgagccttcgcgtaccagcgcatttactgcgatgacggcgatccggcatgccggaggttttgtcagcttcgatcctaatattcgtgaagatctatggcaagacgagcatttgctccgcttgtgtttgcggcaggcgctacaactggcggatgtcgtcaagctctcggaagaagaatggcgacttatcagtggaaaaacacagaacgatcaggatatatgcgccctggcaaaagagtatgagatcgccatgctgttggtgactaaaggtgcagaaggggtggtggtctgttatcgaggacaagttcaccattttgctggaatgtctgtgaattgtgtcgatagcacgggggcgggagatgcgttcgttgccgggttactcacaggtctgtcctctacgggattatctacagatgagagagaaatgcgacgaattatcgatctcgctcaacgttgcggagcgcttgcagtaacggcgaaaggggcaatgacagcgctgccatgtcgacaagaactggaatag

SEQ ID NO: 48 - protein, 304 aa, Escherichia coli
MSAKVWVLGDAVVDLLPESDGRLLPCPGGAPANVAVGIARLGGTSGFIGRVGDDPFGALMQRTLLTEGVDITYLKQDEWHRTSTVLVDLNDQGERSFTFMVRPSADLFLETTDLPCWRHGEWLHLCSIALSAEPSRTSAFTAMTAIRHAGGFVSFDPNIREDLWQDEHLLRLCLRQALQLADVVKLSEEEWRLISGKTQNDQDICALAKEYEIAMLLVTKGAEGVVVCYRGQVHHFAGMSVNCVDSTGAGDAFVAGLLTGLSSTGLSTDEREMRRIIDLAQRCGALAVTAKGAMTALPCRQELE

SEQ ID NO: 49 - DNA, 879 bp, Enterococcus faecalis
atgacagaaaaacttttaggaagtatcgaagccggtggcacaaaatttgtatgtggcgttgggacagatgatttgaccatcgtagaacgtgtcagttttcccacaacaaccccagaagaaacaatgaaaaaagtaatagaatttttccaacaatatcctttaaaagcgattgggattggttcatttggtccgattgatattcacgttgattctcctacgtatggttatatcacttctacaccaaaattagcttggcgtaactttgacttgttaggaactatgaaacaacattttgatgtgccaatggcttggacaacggatgtgaatgctgcggcatatggtgagtatgttgctggaaatgggcaacatacatctagttgtgtatattatacaattggaactggtgttggcgctggagcgattcaaaacggtgagtttattgaaggctttagccacccagaaatggggcatgcgttagttcgtcgtcatcctgaagatacgtatgcaggaaattgtccttatcatggagattgtttagaagggattgcagcaggaccagcagttgaaggtcgttctggtaaaaaaggacatttattggaagaggatcataaaacttgggaattagaagcttattatttagcgcaagcggcgtacaatacgactttattattagcgccagaagtgatcattttaggtggcggcgtcatgaaacaacgtcatttgatgccgaaagttcgtgaaaaatttgctgaattagtcaatggatatgtggaaacaccgcctttagaaaaatacttggtgacgcctcttttagaagataatccaggaacaatcggttgctttgccttggcaaaaaaagctttaatggctcaaaaataa

SEQ ID NO: 50 - protein, 292 aa, Enterococcus faecalis
MTEKLLGSIEAGGTKFVCGVGTDDLTIVERVSFPTTTPEETMKKVIEFFQQYPLKAIGIGSFGPIDIHVDSPTYGYITSTPKLAWRNFDLLGTMKQHFDVPMAWTTDVNAAAYGEYVAGNGQHTSSCVYYTIGTGVGAGAIQNGEFIEGFSHPEMGHALVRRHPEDTYAGNCPYHGDCLEGIAAGPAVEGRSGKKGHLLEEDHKTWELEAYYLAQAAYNTTLLLAPEVIILGGGVMKQRHLMPKVREKFAELVNGYVETPPLEKYLVTPLLEDNPGTIGCFALAKKALMAQK

SEQ ID NO: 51 - DNA, 1458 bp, Saccharomyces cerevisiae
ttaagcgccaatgataccaagagacttaccttcggcaattcttttttcggacaatgcagcaataacagcagcacctgcacctgaaccatcctcagctggaacaatcgtaattggatctttgcttgcgtcaccagtccatccatagatatctctcaaacccttagcggcggcttccttgaaacctgggtatttgttatagacagaaccgtcagcggcaatgtgaccagtcttgtaacctctcttttggcaaatagcggcaataccacaaacagctaatctagcagctctggtaccgatcaattcacaaagtcttctaatcaacttacgttctggcagagtggtcttgacaccaaagtccttttggaagatgtcatcagtatcttccaagttttcaaatggatcatcctcgattcttgctgggtaggaggtatccatgatgtatggttgtttcaacttgcttagatcttgatccttcaacatcaagcccttctcgtttaattcaagtaacactagacgcaacaattcacccaagtagtaaccggaggtcatcttttcaaaagcttgttgaccaggtcttggagattgttcgtcgacagcaacatcgtacttggttcttggcaagaccaaatgttcattatcgaaggaaccatattcacaattgatagccattggagagttacttggaatatcgtctgctaatttgccctccaacttttcgatatcggaaacaacatcatagaaagcaccgttgacaccagtaccgaaaatcacacccatcttagtctctgggtcagtgtagtatgaggcaattaaagtaccaacagtatcattaatcaatgctacaatttcaataggcaactctctcttggaaatttcgttttgtagcaatgggacgacatcgtggccttcgacatttggaatatcgaaacccttggtccatctttgcaaaataccttcgttaatcttgttttgggaagctgggtacgagaaggtgaaacctaatggtaaggtgtccttggtgtttagcaattcttgctcgaccataaagtccttcaaagagtcggcaataaaggaccataactcctcttggtgcttagtggttctcatgtcatgtggtagtttatacttggattgagtggtgtcaaaggtatggttaccgctcaacttgaccaacacgactcttaagttagtaccacccaaatcaatggccaaatagttaccagattctttacctgttgggaattccatgacccaaccgggaatcattggaatgttacctcccttctttgtcaaacctttattcaattcgtcgataaagtgcttaacaacctttctcaaggtctcgctgtcaactgtaaacatatcttccaactgatgaatttcatccatcaattccttgggcacatcagccatggaaccctttctagcctgtggtttctttggacctaaatgaaccat

SEQ ID NO: 52 - protein, 485 aa, Saccharomyces cerevisiae
MVHLGPKKPQARKGSMADVPKELMDEIHQLEDMFTVDSETLRKVVKHFIDELNKGLTKKGGNIPMIPGWVMEFPTGKESGNYLAIDLGGTNLRVVLVKLSGNHTFDTTQSKYKLPHDMRTTKHQEELWSFIADSLKDFMVEQELLNTKDTLPLGFTFSYPASQNKINEGILQRWTKGFDIPNVEGHDVVPLLQNEISKRELPIEIVALINDTVGTLIASYYTDPETKMGVIFGTGVNGAFYDVVSDIEKLEGKLADDIPSNSPMAINCEYGSFDNEHLVLPRTKYDVAVDEQSPRPGQQAFEKMTSGYYLGELLRLVLLELNEKGLMLKDQDLSKLKQPYIMDTSYPARIEDDPFENLEDTDDIFQKDFGVKTTLPERKLIRRLCELIGTRAARLAVCGIAAICQKRGYKTGHIAADGSVYNKYPGFKEAAAKGLRDIYGWTGDASKDPITIVPAEDGSGAGAAVIAALSEKRIAEGKSLGIIGA

SEQ ID NO: 53 - DNA, 1461 bp, Saccharomyces cerevisiae
atggttcatttaggtccaaaaaaaccacaagccagaaagggttccatggccgatgtgccaaaggaattgatgcaacaaattgagaattttgaaaaaattttcactgttccaactgaaactttacaagccgttaccaagcacttcatttccgaattggaaaagggtttgtccaagaagggtggtaacattccaatgattccaggttgggttatggatttcccaactggtaaggaatccggtgatttcttggccattgatttgggtggtaccaacttgagagttgtcttagtcaagttgggcggtgaccgtacctttgacaccactcaatctaagtacagattaccagatgctatgagaactactcaaaatccagacgaattgtgggaatttattgccgactctttgaaagcttttattgatgagcaattcccacaaggtatctctgagccaattccattgggtttcaccttttctttcccagcttctcaaaacaaaatcaatgaaggtatcttgcaaagatggactaaaggttttgatattccaaacattgaaaaccacgatgttgttccaatgttgcaaaagcaaatcactaagaggaatatcccaattgaagttgttgctttgataaacgacactaccggtactttggttgcttcttactacactgacccagaaactaagatgggtgttatcttcggtactggtgtcaatggtgcttactacgatgtttgttccgatatcgaaaagctacaaggaaaactatctgatgacattccaccatctgctccaatggccatcaactgtgaatacggttccttcgataatgaacatgtcgttttgccaagaactaaatacgatatcaccattgatgaagaatctccaagaccaggccaacaaacctttgaaaaaatgtcttctggttactacttaggtgaaattttgcgtttggccttgatggacatgtacaaacaaggtttcatcttcaagaaccaagacttgtctaagttcgacaagcctttcgtcatggacacttcttacccagccagaatcgaggaagatccattcgagaacctagaagataccgatgacttgttccaaaatgagttcggtatcaacactactgttcaagaacgtaaattgatcagacgtttatctgaattgattggtgctagagctgctagattgtccgtttgtggtattgctgctatctgtcaaaagagaggttacaagaccggtcacatcgctgcagacggttccgtttacaacagatacccaggtttcaaagaaaaggctgccaatgctttgaaggacatttacggctggactcaaacctcactagacgactacccaatcaagattgttcctgctgaagatggttccggtgctggtgccgctgttattgctgctttggcccaaaaaagaattgctgaaggtaagtccgttggtatcatcggtgcttaa

SEQ ID NO: 54 - protein, 486 aa, Saccharomyces cerevisiae
MVHLGPKKPQARKGSMADVPKELMQQIENFEKIFTVPTETLQAVTKHFISELEKGLSKKGGNIPMIPGWVMDFPTGKESGDFLAIDLGGTNLRVVLVKLGGDRTFDTTQSKYRLPDAMRTTQNPDELWEFIADSLKAFIDEQFPQGISEPIPLGFTFSFPASQNKINEGILQRWTKGFDIPNIENHDVVPMLQKQITKRNIPIEVVALINDTTGTLVASYYTDPETKMGVIFGTGVNGAYYDVCSDIEKLQGKLSDDIPPSAPMAINCEYGSFDNEHVVLPRTKYDITIDEESPRPGQQTFEKMSSGYYLGEILRLALMDMYKQGFIFKNQDLSKFDKPFVMDTSYPARIEEDPFENLEDTDDLFQNEFGINTTVQERKLIRRLSELIGARAARLSVCGIAAICQKRGYKTGHIAADGSVYNRYPGFKEKAANALKDIYGWTQTSLDDYPIKIVPAEDGSGAGAAVIAALAQKRIAEGKSVGIIGA

SEQ ID NO: 55 - DNA, 1374 bp, Escherichia coli
ggtaacactttctatccccattttcacctcgcgcctcctgccgggtggatgaacgatccaaacggcctgatctggtttaacgatcgttatcacgcgttttatcaacatcacccgatgagcgaacactgggggccaatgcactggggacatgccaccagcgacgatatgatccactggcagcatgagcctattgcgctagcgccaggagacgagaatgacaaagacgggtgtttttcaggtagtgctgtcgatgacaatggtgtcctctcacttatctacaccggacacgtctggctcgatggtgcaggtaatgacgatgcaattcgcgaagtacaatgtctggctaccagtcgggatggtattcatttcgagaaacagggtgtgatcctcactccaccagaaggcatcatgcacttccgcgatcctaaagtgtggcgtgaagccgacacatggtggatggtagtcggggcgaaagacccaggcaacacggggcagatcctgctttatcgcggcagttcattgcgtgaatggactttcgatcgcgtactggcccacgctgatgcgggtgaaagctatatgtgggaatgtccggactttttcagccttggcgatcagcattatctgatgttttccccgcagggaatgaatgccgagggatacagttatcgaaatcgctttcaaagtggcgtaatacccggaatgtggtcgccaggacgactttttgcacaatccgggcattttactgaacttgataacgggcatgacttttatgcaccacaaagctttgtagcgaaggatggtcggcgtattgttatcggctggatggatatgtgggaatcgccaatgccctcaaaacgtgaaggctgggcaggctgcatgacgctggcgcgcgagctatcagagagcaatggcaaactcctacaacgcccggtacacgaagctgagtcgttacgccagcagcatcaatctatctctccccgcacaatcagcaataaatatgttttgcaggaaaacgcgcaagcagttgagattcagttgcagtgggcgctgaagaacagtgatgccgaacattacggattacagctcggcgctggaatgcggctgtatattgataaccaatctgagcgacttgttttgtggcggtattacccacacgagaatttagatggctaccgtagtattcccctcccgcagggtgacatgctcgccctaaggatatttatcgatacatcatccgtggaagtatttattaacgacggggaggcggtgatgagtagccgaatatatccgcagccagaagaacgggaactgtcgctctatgcctcccacggagtggctgtgctgcaacatggagcactctggcaactgggttaa

SEQ ID NO: 56 - protein, 477 aa, Escherichia coli
MTQSRLHAAQNALAKLHERRGNTFYPHFHLAPPAGWMNDPNGLIWFNDRYHAFYQHHPMSEHWGPMHWGHATSDDMIHWQHEPIALAPGDENDKDGCFSGSAVDDNGVLSLIYTGHVWLDGAGNDDAIREVQCLATSRDGIHFEKQGVILTPPEGIMHFRDPKVWREADTWWMVVGAKDPGNTGQILLYRGSSLREWTFDRVLAHADAGESYMWECPDFFSLGDQHYLMFSPQGMNAEGYSYRNRFQSGVIPGMWSPGRLFAQSGHFTELDNGHDFYAPQSFVAKDGRRIVIGWMDMWESPMPSKREGWAGCMTLARELSESNGKLLQRPVHEAESLRQQHQSISPRTISNKYVLQENAQAVEIQLQWALKNSDAEHYGLQLGAGMRLYIDNQSERLVLWRYYPHENLDGYRSIPLPQGDMLALRIFIDTSSVEVFINDGEAVMSSRIYPQPEERELSLYASHGVAVLQHGALWQLG

SEQ ID NO: 57 - DNA, 1434 bp, Escherichia coli
atgacgcaatctcgattgcatgcggcgcaaaacgccctagcaaaacttcatgagcaccggggtaacactttctatccccattttcacctcgcgcctcctgccgggtggatgaacgatccaaacggcctgatctggtttaacgatcgttatcacgcgttttatcaacatcatccgatgagcgaacactgggggccaatgcactggggacatgccaccagcgacgatatgatccactggcagcatgagcctattgcgctagcgccaggagacgataatgacaaagacgggtgtttttcaggtagtgctgtcgatgacaatggtgtcctctcacttatctacaccggacacgtctggctcgatggtgcaggtaatgacgatgcaattcgcgaagtacaatgtctggctaccagtcgggatggtattcatttcgagaaacagggtgtgatcctcactccaccagaaggaatcatgcacttccgcgatcctaaagtgtggcgtgaagccgacacatggtggatggtagtcggggcgaaagatccaggcaacacggggcagatcctgctttatcgcggcagttcgttgcgtgaatggaccttcgatcgcgtactggcccacgctgatgcgggtgaaagctatatgtgggaatgtccggactttttcagccttggcgatcagcattatctgatgttttccccgcagggaatgaatgccgagggatacagttaccgaaatcgctttcaaagtggcgtaatacccggaatgtggtcgccaggacgactttttgcacaatccgggcattttactgaacttgataacgggcatgacttttatgcaccacaaagctttttagcgaaggatggtcggcgtattgttatcggctggatggatatgtgggaatcgccaatgccctcaaaacgtgaaggatgggcaggctgcatgacgctggcgcgcgagctatcagagagcaatggcaaacttctacaacgcccggtacacgaagctgagtcgttacgccagcagcatcaatctgtctctccccgcacaatcagcaataaatatgttttgcaggaaaacgcgcaagcagttgagattcagttgcagtgggcgctgaagaacagtgatgccgaacattacggattacagctcggcactggaatgcggctgtatattgataaccaatctgagcgacttgttttgtggcggtattacccacacgagaatttagacggctaccgtagtattcccctcccgcagcgtgacacgctcgccctaaggatatttatcgatacatcatccgtggaagtatttattaacgacggggaagcggtgatgagtagtcgaatctatccgcagccagaagaacgggaactgtcgctttatgcctcccacggagtggctgtgctgcaacatggagcactctggctactgggttaa

SEQ ID NO: 58 - protein, 477 aa, Escherichia coli
MTQSRLHAAQNALAKLHEHRGNTFYPHFHLAPPAGWMNDPNGLIWFNDRYHAFYQHHPMSEHWGPMHWGHATSDDMIHWQHEPIALAPGDDNDKDGCFSGSAVDDNGVLSLIYTGHVWLDGAGNDDAIREVQCLATSRDGIHFEKQGVILTPPEGIMHFRDPKVWREADTWWMVVGAKDPGNTGQILLYRGSSLREWTFDRVLAHADAGESYMWECPDFFSLGDQHYLMFSPQGMNAEGYSYRNRFQSGVIPGMWSPGRLFAQSGHFTELDNGHDFYAPQSFLAKDGRRIVIGWMDMWESPMPSKREGWAGCMTLARELSESNGKLLQRPVHEAESLRQQHQSVSPRTISNKYVLQENAQAVEIQLQWALKNSDAEHYGLQLGTGMRLYIDNQSERLVLWRYYPHENLDGYRSIPLPQRDTLALRIFIDTSSVEVFINDGEAVMSSRIYPQPEERELSLYASHGVAVLQHGALWLLG

SEQ ID NO: 59 - DNA, 1599 bp, Bifidobacterium lactis
atggcaacccttcccaccaatattcccgccaacggcattctgacccccgacccggcgctcgaccctgtgctcacgccgatctcggaccatgccgagcagctgtcactcgccgaagcaggcgtgtcggcactggaaaccacccgcaacgaccgctggtacccgaagttccacattgcctccaatggcgggtggatcaacgacccgaacggcctgtgccgctacaacggacgctggcacgtgttctaccagctgcatccccacggcacacagtggggcccgatgcattggggccacgtctcctccgacaacatggtcgactggcaccgcgaacccatcgccttcgcgccaagcctcgaacaggaacgccacggtgtgttctccggttccgccgtgattggcgacgacggcaagccgtggattttctacaccggccaccgctgggccaacggcaaggacaacaccggaggcgactggcaggtgcagatgctcgccaagccgaacgacgacgaactgaagaccttcacgaaggagggcatgatcatcgactgccccaccgacgaggtggaccaccacttccgcgacccgaaggtgtggaagaccggtgacacctggtatatgaccttcggtgtctcgtcgaaggagcatcgtggccagatgtggctgtacacgtcgagcgacatggtgcactggagcttcgatcgggtgctgttcgagcatccggatccgaacgtgttcatgcttgaatgccccgatttcttcccgatccgcgatgcgcggggcaacgagaaatgggtcatcggcttctccgcgatgggtgccaagccaaatggcttcatgaaccgcaacgtgaacaatgccggctacatggtgggcacatggaagccaggcgagagcttcaagccggagaccgagttccgcctgtgggacgaaggccataacttctatgcaccacagtcgttcaacaccgaagggcgccagatcatgtacggctggatgagcccgttcgtcgcccccatcccgatggaggaggacggctggtgcggcaacctcaccctcccccgcgagatcacgctgggcgatgacggtgacctggtcaccgcccccaccatcgaaatggaggggctgcgcgagaataccataggcttcgactcgctcgaccttggtacgaaccagacctccacgatcctcgacgatgacggcggcgccctggaaatcgagatgagactcgatctgaacaaaaccaccgccgaacgcgccggactgcatgtgcatgccacaagcgacggccactacacggcaatcgtattcgacgcgcagatcggcggcgtcgtcatcgaccggcagaacgtggcgaacggagacaaaggctaccgggtggccaagctcagcgacaccgagctcgcagccgatacgcttgacttgcgcgtgttcatcgaccgcggatgcgtcgaggtctacgtcgacggcggcaagcatgcgatgagctcgtactcgttccctggcgatggcgcacgcgccgtcgaactcgtgagcgaatccggcaccacgcacatcgacaccctcaccatgcactcgctcaagtccatcggactcgagtga

SEQ ID NO: 60 - protein, 532 aa, Bifidobacterium lactis
MATLPTNIPANGILTPDPALDPVLTPISDHAEQLSLAEAGVSALETTRNDRWYPKFHIASNGGWINDPNGLCRYNGRWHVFYQLHPHGTQWGPMHWGHVSSDNMVDWHREPIAFAPSLEQERHGVFSGSAVIGDDGKPWIFYTGHRWANGKDNTGGDWQVQMLAKPNDDELKTFTKEGMIIDCPTDEVDHHFRDPKVWKTGDTWYMTFGVSSKEHRGQMWLYTSSDMVHWSFDRVLFEHPDPNVFMLECPDFFPIRDARGNEKWVIGFSAMGAKPNGFMNRNVNNAGYMVGTWKPGESFKPETEFRLWDEGHNFYAPQSFNTEGRQIMYGWMSPFVAPIPMEEDGWCGNLTLPREITLGDDGDLVTAPTIEMEGLRENTIGFDSLDLGTNQTSTILDDDGGALEIEMRLDLNKTTAERAGLHVHATSDGHYTAIVFDAQIGGVVIDRQNVANGDKGYRVAKLSDTELAADTLDLRVFIDRGCVEVYVDGGKHAMSSYSFPGDGARAVELVSESGTTHIDTLTMHSLKSIGLE

SEQ ID NO: 61 - DNA, 1599 bp, Saccharomyces cerevisiae
atgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttggatccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatag

SEQ ID NO: 62 - protein, 532 aa, Saccharomyces cerevisiae
MLLQAFLFLLAGFAAKISASMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 63 - DNA, 1302 bp, Corynebacterium glutamicum
gtgtgtggggctatgcacacagaactttccagtttgcgccctgcgtaccatgtgactcctccgcagggcaggctcaatgatcccaacggaatgtacgtcgatggcgataccctccacgtctactaccagcacgatccaggtttccccttcgcaccaaagcgcaccggctgggctcacaccaccacgccgttgaccggaccgcagcgattgcagtggacgcacctgcccgacgctctttacccggatgcatcctatgacctggatggatgctattccggtggagccgtatttactgacggcacacttaaacttttctacaccggcaacctaaaaattgacggcaagcgccgcgccacccaaaacctcgtcgaagtcgaggacccaactgggctgatgggcggcattcatcgccgttcgcctaaaaatccgcttatcgacggacccgccagcggtttcacaccccattaccgcgatcccatgatcagccctgatggtgatggttggaaaatggttcttggggcccaacgcgaaaacctcaccggtgcagcggttctataccgctcgacagatcttgaaaactgggaattctccggtgaaatcacctttgacctcagtgatgcacaacctggttctgctcctgatctcgttcccggtggctacatgtgggaatgccccaacctttttacgcttcgcgatgaagaaactggcgaagatctcgacgtgctgattttctgtccacaaggattggaccgaatccacgatgaggttactcactacgcaagctctgaccagtgcggatatgtcgtcggcaagcttgaaggaacgaccttccgcgtcttgcgaggattcagcgagctggatttcggccatgaattctacgcaccgcaggttgcagtaaacggttctgatgcctggctcgtgggctggatggggctgcccgcgcaggatgatcacccaacagttgcacgggaaggatgggtgcactgcctgactgtgccccgcaagcttcatttgcgcaaccacgcgatctatcaagagcttcttctcccagagggggagtcaggggtaatcagatctgtattaggttctgaacctgtccgagtagacatccgaggcaatatttccctcgagtgggatggtgtccgtttgtctgtggatcgtggtggtgatcgtcgcgtagctgaggtaaaacctggcgaattagtgatcgcggacgataatacagccattgagataactgcaggtgatggacaggtttcattcgctttccgggctttcaaaggtgacactattgagagataa

SEQ ID NO: 64 - protein, 433 aa, Corynebacterium glutamicum
MCGAMHTELSSLRPAYHVTPPQGRLNDPNGMYVDGDTLHVYYQHDPGFPFAPKRTGWAHTTTPLTGPQRLQWTHLPDALYPDASYDLDGCYSGGAVFTDGTLKLFYTGNLKIDGKRRATQNLVEVEDPTGLMGGIHRRSPKNPLIDGPASGFTPHYRDPMISPDGDGWKMVLGAQRENLTGAAVLYRSTDLENWEFSGEITFDLSDAQPGSAPDLVPGGYMWECPNLFTLRDEETGEDLDVLIFCPQGLDRIHDEVTHYASSDQCGYVVGKLEGTTFRVLRGFSELDFGHEFYAPQVAVNGSDAWLVGWMGLPAQDDHPTVAREGWVHCLTVPRKLHLRNHAIYQELLLPEGESGVIRSVLGSEPVRVDIRGNISLEWDGVRLSVDRGGDRRVAEVKPGELVIADDNTAIEITAGDGQVSFAFRAFKGDTIER

SEQ ID NO: 65 - DNA, 1473 bp, Leuconostoc mesenteroides
atggaaattcaaaacaaagcaatgttgatcacttatgctgattcgttgggcaaaaacttaaaagatgttcatcaagtcttgaaagaagatattggagatgcgattggtggggttcatttgttgcctttcttcccttcaacaggtgatcgcggttttgcgccagccgattatactcgtgttgatgccgcatttggtgattgggcagatgtcgaagcattgggtgaagaatactatttgatgtttgacttcatgattaaccatatttctcgtgaatcagtgatgtatcaagattttaagaagaatcatgacgattcaaagtataaagatttctttattcgttgggaaaagttctgggcaaaggccggcgaaaaccgtccaacacaagccgatgttgacttaatttacaagcgtaaagataaggcaccaacgcaagaaatcacttttgatgatggcacaacagaaaacttgtggaatacttttggtgaagaacaaattgacattgatgttaattcagccattgccaaggaatttattaagacaacccttgaagacatggtaaaacatggtgctaacttgattcgtttggatgcctttgcgtatgcagttaaaaaagttgacacaaatgacttcttcgttgagccagaaatctgggacactttgaatgaagtacgtgaaattttgacaccattaaaggctgaaattttaccagaaattcatgaacattactcaatccctaaaaagatcaatgatcatggttacttcacctatgactttgcattaccaatgacaacgctttacacattgtattcaggtaagacaaatcaattggcaaagtggttgaagatgtcaccaatgaagcaattcacaacattggacacgcatgatggtattggtgtcgttgatgcccgtgatattctaactgatgatgaaattgactacgcttctgaacaactttacaaggttggcgcgaatgtcaaaaagacatattcatctgcttcatacaacaaccttgatatttaccaaattaactcaacttattattcagcattgggaaatgatgatgcagcatacttgttgagtcgtgtcttccaagtctttgcgcctggaattccacaaatttattacgttggtttgttggcaggtgaaaacgatatcgcgcttttggagtcaactaaagaaggtcgtaatattaaccgtcattactatacgcgtgaagaagttaagtcagaagttaagcgaccagttgttgctaacttattgaagctattgtcatggcgtaatgaaagccctgcatttgatttggctggctcaatcacagttgacacgccaactgatacaacaattgtggtgacacgtcaagatgaaaatggtcaaaacaaagctgtattaacagccgatgcggccaacaaaacttttgaaatcgttgagaatggtcaaactgttatgagcagtgataatttgactcagaactaa

SEQ ID NO: 66 - protein, 490 aa, Leuconostoc mesenteroides
MEIQNKAMLITYADSLGKNLKDVHQVLKEDIGDAIGGVHLLPFFPSTGDRGFAPADYTRVDAAFGDWADVEALGEEYYLMFDFMINHISRESVMYQDFKKNHDDSKYKDFFIRWEKFWAKAGENRPTQADVDLIYKRKDKAPTQEITFDDGTTENLWNTFGEEQIDIDVNSAIAKEFIKTTLEDMVKHGANLIRLDAFAYAVKKVDTNDFFVEPEIWDTLNEVREILTPLKAEILPEIHEHYSIPKKINDHGYFTYDFALPMTTLYTLYSGKTNQLAKWLKMSPMKQFTTLDTHDGIGVVDARDILTDDEIDYASEQLYKVGANVKKTYSSASYNNLDIYQINSTYYSALGNDDAAYLLSRVFQVFAPGIPQIYYVGLLAGENDIALLESTKEGRNINRHYYTREEVKSEVKRPVVANLLKLLSWRNESPAFDLAGSITVDTPTDTTIVVTRQDENGQNKAVLTADAANKTFEIVENGQTVMSSDNLTQN

SEQ ID NO: 67 - DNA, 1515 bp, Bifidobacterium adolescentis
atgaaaaacaaggtgcagctcatcacttacgccgaccgccttggcgacggcaccatcaagtcgatgaccgacattctgcgcacccgcttcgacggcgtgtacgacggcgttcacatcctgccgttcttcaccccgttcgacggcgccgacgcaggcttcgacccgatcgaccacaccaaggtcgacgaacgtctcggcagctgggacgacgtcgccgaactctccaagacccacaacatcatggtcgacgccatcgtcaaccacatgagttgggaatccaagcagttccaggacgtgctggccaagggcgaggagtccgaatactatccgatgttcctcaccatgagctccgtgttcccgaacggcgccaccgaagaggacctggccggcatctaccgtccgcgtccgggcctgccgttcacccactacaagttcgccggcaagacccgcctcgtgtgggtcagcttcaccccgcagcaggtggacatcgacaccgattccgacaagggttgggaatacctcatgtcgattttcgaccagatggccgcctctcacgtcagctacatccgcctcgacgccgtcggctatggcgccaaggaagccggcaccagctgcttcatgaccccgaagaccttcaagctgatctcccgtctgcgtgaggaaggcgtcaagcgcggtctggaaatcctcatcgaagtgcactcctactacaagaagcaggtcgaaatcgcatccaaggtggaccgcgtctacgacttcgccctgcctccgctgctgctgcacgcgctgagcaccggccacgtcgagcccgtcgcccactggaccgacatacgcccgaacaacgccgtcaccgtgctcgatacgcacgacggcatcggcgtgatcgacatcggctccgaccagctcgaccgctcgctcaagggtctcgtgccggatgaggacgtggacaacctcgtcaacaccatccacgccaacacccacggcgaatcccaggcagccactggcgccgccgcatccaatctcgacctctaccaggtcaacagcacctactattcggcgctcgggtgcaacgaccagcactacatcgccgcccgcgcggtgcagttcttcctgccgggcgtgccgcaagtctactacgtcggcgcgctcgccggcaagaacgacatggagctgctgcgtaagacgaataacggccgcgacatcaatcgccattactactccaccgcggaaatcgacgagaacctcaagcgtccggtcgtcaaggccctgaacgcgctcgccaagttccgcaacgagctcgacgcgttcgacggcacgttctcgtacaccaccgatgacgacacgtccatcagcttcacctggcgcggcgaaaccagccaggccacgctgacgttcgagccgaagcgcggtctcggtgtggacaacactacgccggtcgccatgttggaatgggaggattccgcgggagaccaccgttcggatgatctgatcgccaatccgcctgtcgtcgcctga

SEQ ID NO: 68 - protein, 504 aa, Bifidobacterium adolescentis [sequence omitted]

SEQ ID NO: 69 - DNA, 1164 bp, Klebsiella pneumoniae
tcagaatgcctggcggaaaatcgcggcaatctcctgctcgttgcctttacgcgggttcgagaacgcattgccgtctttcagagccatctccgccatgtaggggaagtcggcctcttttactcccagatcgcgcagatgctgcggaataccgatatccatcgacagacgcgtgatagcggcgatggctttttccgccgcgtcgagagtggacagtccggtgatattttcgcccatcagttcagcgatatcggcgaatttctccgggttggcgatcaggttgtagcgggccacatgcggcagcaggacagcgttggccacgccgtgcggcatgtcgtacaggccgcccagctggtgcgccatggcgtgcacgtagccgaggttggcgttattgaaagccatcccggccagcagagaggcataggccatgttttcccgcgcctgcagattgctgccgagggccacggcctggcgcaggttgcgggcgatgaggcggatcgcctgcatggcggcggcgtccgtcaccgggttagcgtctttggagatataggcctctacggcgtgggtcagggcatccatcccggtcgccgcggtcagggcggccggtttaccgatcatcagcagcggatcgttgatagagaccgacggcaggttgcgccagctgacgatcacaaacttcactttggtttcggtgttggtcaggacgcagtggcgggtgacctcgctggcggtgccggcggtggtattgaccgcgacgataggcggcagcgggttggtcagggtctcgattccggcatactggtacagatcgccctcatgggtggcggcgatgccgatgcctttgccgcaatcgtgcgggctgccgccgcccacggtgacgatgatgtcgcactgttcgcggcgaaacacggcgaggccgtcgcgcacgttggtgtctttcgggttcggctcgacgccgtcaaagatcgccacctcgatcccggcctcccgcagataatgcagggttttgtccactgcgccatctttaattgcccgcaggcctttgtcggtgaccagcagggcttttttcccccccagcagctggcagcgttcgccgactacggaaatggcgttggggccaaaaaagttaacgtttggcaccagataatcaaacatacgatagctcat

SEQ ID NO: 70 - protein, 387 aa, Klebsiella pneumoniae
MSYRMFDYLVPNVNFFGPNAISVVGERCQLLGGKKALLVTDKGLRAIKDGAVDKTLHYLREAGIEVAIFDGVEPNPKDTNVRDGLAVFRREQCDIIVTVGGGSPHDCGKGIGIAATHEGDLYQYAGIETLTNPLPPIVAVNTTAGTASEVTRHCVLTNTETKVKFVIVSWRNLPSVSINDPLLMIGKPAALTAATGMDALTHAVEAYISKDANPVTDAAAMQAIRLIARNLRQAVALGSNLQARENMAYASLLAGMAFNNANLGYVHAMAHQLGGLYDMPHGVANAVLLPHVARYNLIANPEKFADIAELMGENITGLSTLDAAEKAIAAITRLSMDIGIPQHLRDLGVKEADFPYMAEMALKDGNAFSNPRKGNEQEIAAIFRQAF

SEQ ID NO: 71 - DNA, 1824 bp, Klebsiella pneumoniae
atgccgttaatagccgggattgatatcggcaacgccaccaccgaggtggcgctggcgtccgactacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttccgcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaa

SEQ ID NO: 72 - DNA, 13669 bp, Artificial Sequence
  Plasmid
tagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattcatttaaatagtcaaaagcctccgaccggaggcttttgactgctaggcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccaagcttgcgggagagaatgatgaacaagagccaacaagttcagacaatcaccctggccgccgcccagcaaatggcggcggcggtggaaaaaaaagccactgagatcaacgtggcggtggtgttttccgtagttgaccgcggaggcaacacgctgcttatccagcggatggacgaggccttcgtctccagctgcgatatttccctgaataaagcctggagcgcctgcagcctgaagcaaggtacccatgaaattacgtcagcggtccagccaggacaatctctgtacggtctgcagctaaccaaccaacagcgaattattatttttggcggcggcctgccagttatttttaatgagcaggtaattggcgccgtcggcgttagcggcggtacggtcgagcaggatcaattattagcccagtgcgccctggattgtttttccgcattataacctgaagcgagaaggtatattatgagctatcgtatgttccgccaggcattctgagtgttaacgaggggaccgtcatgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtatcgggctcagcgcgtccggcgagatagccctcactcatgcccagctgccggcggacgcgccgctggctaccggacacgtcaccgatagcgacgatcaactgcgtacgctcggcgccaacgccgggcagctggttaaagtcctgccgttaagtgagagaaactgaatgtatcgtatctatacccgcaccggggataaaggcaccaccgccctgtacggcggcagccgcatcgagaaagaccatattcgcgtcgaggcctacggcaccgtcgatgaactgatatcccagctgggcgtctgctacgccacgacccgcgacgccgggctgcgggaaagcctgcaccatattcagcagacgctgttcgtgctgggggctgaactggccagcgatgcgcggggcctgacccgcctgagccagacgatcggcgaagaggagatcaccgccctggagcggcttatcgaccgcaatatggccgagagcggcccgttaaaacagttcgtgatcccggggaggaatctcgcctctgcccagctgcacgtggcgcgcacccagtcccgtcggctcgaacgcctgctgacggccatggaccgcgcgcatccgctgcgcgacgcgctcaaacgctacagcaatcgcctgtcggatgccctgttctccatggcgcgaatcgaagagactaggcctgatgcttgcgcttgaactggcctagcaaacacagaaaaaagcccgcacctgacagtgcgggctttttttttcctaggcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccaagcttcaccttttgagccgatgaacaatgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgaactggacggcaaacgccgggaccagtttgacatgatcgaccgatttatcgccgattacgcgatcaacgttgagcgcacagagcaggcaatgcgcctggaggcggtggaaatagcccgtatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctacgcgccgtttaacgccctggcgctgttggtcggttcgcagtgcggccgccccggcgtgttgacgcagtgctcggtggaagaggccaccgagctggagctgggcatgcgtggcttaaccagctacgccgagacggtgtcggtctacggcaccgaagcggtatttaccgacggcgatgatacgccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattactaaaggcgccggggttcagggactgcaaaacggcgcggtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacatcctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcggcagttcgaggtggtgagtgcggtcaacgacatcaatgactatcaggggccgggcaccggctatcgcatctctgccgaacgctgggcggagatcaaaaatattccgggcgtggttcagcccgacaccattgaataaggcggtattcctgtgcaacagacaacccaaattcagccctcttttaccctgaaaacccgcgagggcggggtagcttctgccgatgaacgcgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgctgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacatcgacttagtaagggagtgaccatgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaagcggaggtcagcatgccgttaatagccgggattgatatcggcaacgccaccaccgaggtggcgctggcgtccgactacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttccgcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaaacgggcgctcgcgccagcctctaggtacaaataaaaaaggcacgtcagatgacgtgccttttttcttgtctagagtactggcgaaagggggatgtgctgcaaggcgattaagttgggtaacgccagggttttcccagtcacgacgttgtaaaacgacggccagtgaattcgagctcggtacccggggcggccgcgctagcgcccgatccagctggagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgcatgcctgcagcccgggttaccatttcaacagatcgtccttagcatataagtagtcgtcaaaaatgaattcaacttcgtctgtttcggcattgtagccgccaactctgatggattcgtggtttttgacaatgatgtcacagcctttttcctttaggaagtccaagtcgaaagtagtggcaataccaatgatcttacaaccggcggcttttccggcggcaatacctgctggagcgtcttcaaatactactaccttagatttggaagggtcttgctcattgatcggatatcctaagccattcctgcccttcagatatggttctggatgaggcttaccctgtttgacatcattagcggtaatgaagtactttggtctcctgattcccagatgctcgaaccatttttgtgccatatcacgggtaccggaagttgccacagcccatttctcttttggtagagcgttcaaagcgttgcacagcttaactgcacctgggacttcaatggatttttcaccgtacttgaccggaatttcagcttctaatttgttaacatactcttcattggcaaagtctggagcgaacttagcaatggcatcaaacgttctccaaccatgcgagacttggataacgtgttcagcatcgaaataaggtttgtccttaccgaaatccctccagaatgcagcaatggctggttgagagatgataatggtaccgtcgacgtcgaacaaagcggcgttaactttcaaagatagaggtttagtagtcaatcccataattctagtctgtttcctggatccaataaatctaatcttcatgtagatctaattcttcaatcatgtccggcaggttcttcattgggtagttgttgtaaacgatttggtatacggcttcaaataatgggaagtcttcgacagagccacatgtttccaaccattcgtgaacttctttgcaggtaattaaaccttgagcggattggccattcaacaactccttttcacattcccaggcgtccttaccagaagtagccattagcctagcaaccttgacgtttctaccaccagcgcaggtggtgatcaaatcagcaacaccagcagactcttggtagtatgtttcttctctagattctgggaaaaacatttgaccgaatctgatgatctcacccaaaccgactctttggatggcagcagaagcgttgttaccccagcctagaccttcgacgaaaccacaacctaaggcaacaacgttcttcaaagcaccacagatggagataccagcaacatcttcgatgacactaacgtggaagtaaggtctgtggaacaaggcctttagaaccttatggtcgacgtccttgccctcgcctctgaaatcctttggaatgtggtaagcaactgttgtttcagaccagtgttcttgagcgacttcggtggcaatgttagcaccagatagagcaccacattgaatacctagttcctcagtgatgtaagaggatagcaattggacacctttagcaccaacttcaaaaccctttagacaggagatagctctgacgtgtgaatcaacatgacctttcaattggctacagatacggggcaaaaattgatgtggaatgttgaaaacgatgatgtcgacatccttgactgaatcaatcaagtctggattagcaaccaaattgtcgggtagagtgatgccaggcaagtatttcacgttttgatgtctagtatttatgatttcagtcaatttttcaccattgatctcttcttcgaacacccacatttgtactattggagcgaaaacttctgggtatcccttacaattttcggcaaccaccttggcaatagtagtaccccagttaccagatccaatcacagtaaccttgaaaggcttttcggcagccttcaaagaaacagaagaggaacttctctttctaccagcattcaagtggccggaagttaagtttaatctatcagcagcagcagccatggaattgtcctccttactagtcatggtctgtttcctgtgtgaaattgttatccgctcacaattccacacattatacgagccggatgattaattgtcaacagctcatttcagaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcacgctagccagtacatttaaatggtaccctctagtcaaggccttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagct

SEQ ID NO: 73 - DNA, 13543 bp, Artificial Sequence
  Plasmid
tagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattcatttaaatagtcaaaagcctccgaccggaggcttttgactgctaggcgatctgtgctgtttgccacggtatgcagcaccagcgcgagattatgggctcgcacgctcgactgtcggacgggggcactggaacgagaagtcaggcgagccgtcacgcccttgactatgccacatcctgagcaaataattcaaccactaaacaaatcaaccgcgtttcccggaggtaaccaagcttgcgggagagaatgatgaacaagagccaacaagttcagacaatcaccctggccgccgcccagcaaatggcggcggcggtggaaaaaaaagccactgagatcaacgtggcggtggtgttttccgtagttgaccgcggaggcaacacgctgcttatccagcggatggacgaggccttcgtctccagctgcgatatttccctgaataaagcctggagcgcctgcagcctgaagcaaggtacccatgaaattacgtcagcggtccagccaggacaatctctgtacggtctgcagctaaccaaccaacagcgaattattatttttggcggcggcctgccagttatttttaatgagcaggtaattggcgccgtcggcgttagcggcggtacggtcgagcaggatcaattattagcccagtgcgccctggattgtttttccgcattataacctgaagcgagaaggtatattatgagctatcgtatgttccgccaggcattctgagtgttaacgaggggaccgtcatgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtatcgggctcagcgcgtccggcgagatagccctcactcatgcccagctgccggcggacgcgccgctggctaccggacacgtcaccgatagcgacgatcaactgcgtacgctcggcgccaacgccgggcagctggttaaagtcctgccgttaagtgagagaaactgaatgtatcgtatctatacccgcaccggggataaaggcaccaccgccctgtacggcggcagccgcatcgagaaagaccatattcgcgtcgaggcctacggcaccgtcgatgaactgatatcccagctgggcgtctgctacgccacgacccgcgacgccgggctgcgggaaagcctgcaccatattcagcagacgctgttcgtgctgggggctgaactggccagcgatgcgcggggcctgacccgcctgagccagacgatcggcgaagaggagatcaccgccctggagcggcttatcgaccgcaatatggccgagagcggcccgttaaaacagttcgtgatcccggggaggaatctcgcctctgcccagctgcacgtggcgcgcacccagtcccgtcggctcgaacgcctgctgacggccatggaccgcgcgcatccgctgcgcgacgcgctcaaacgctacagcaatcgcctgtcggatgccctgttctccatggcgcgaatcgaagagactaggcctgatgcttgcgcttgaactggcctagcaaacacagaaaaaagcccgcacctgacagtgcgggctttttttttcctaggcgatctgtgctgtttgccacggtatgcagcaccagcgcgagattatgggctcgcacgctcgactgtcggacgggggcactggaacgagaagtcaggcgagccgtcacgcccttgactatgccacatcctgagcaaataattcaaccactaaacaaatcaaccgcgtttcccggaggtaaccaagcttcaccttttgagccgatgaacaatgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgaactggacggcaaacgccgggaccagtttgacatgatcgaccgatttatcgccgattacgcgatcaacgttgagcgcacagagcaggcaatgcgcctggaggcggtggaaatagcccgtatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctacgcgccgtttaacgccctggcgctgttggtcggttcgcagtgcggccgccccggcgtgttgacgcagtgctcggtggaagaggccaccgagctggagctgggcatgcgtggcttaaccagctacgccgagacggtgtcggtctacggcaccgaagcggtatttaccgacggcgatgatacgccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattactaaaggcgccggggttcagggactgcaaaacggcgcggtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacatcctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcggcagttcgaggtggtgagtgcggtcaacgacatcaatgactatcaggggccgggcaccggctatcgcatctctgccgaacgctgggcggagatcaaaaatattccgggcgtggttcagcccgacaccattgaataaggcggtattcctgtgcaacagacaacccaaattcagccctcttttaccctgaaaacccgcgagggcggggtagcttctgccgatgaacgcgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgctgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacatcgacttagtaagggagtgaccatgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaagcggaggtcagcatgccgttaatagccgggattgatatcggcaacgccaccaccgaggtggcgctggcgtccgactacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttccgcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaaacgggcgctcgcgccagcctctaggtacaaataaaaaaggcacgtcagatgacgtgccttttttcttgtctagcgtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgactagtaaggaggacaattccatggctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattagatttattggatccaggaaacagactagaattatgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaacccgggctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactccagctggatcgggcgctagagtatacatttaaatggtaccctctagtcaaggccttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagct

SEQ ID NO: 74 - DNA, 13543 bp, Artificial Sequence
  Plasmid
tagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattcatttaaatagtcaaaagcctccgaccggaggcttttgactgctaggcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccaagcttgcgggagagaatgatgaacaagagccaacaagttcagacaatcaccctggccgccgcccagcaaatggcggcggcggtggaaaaaaaagccactgagatcaacgtggcggtggtgttttccgtagttgaccgcggaggcaacacgctgcttatccagcggatggacgaggccttcgtctccagctgcgatatttccctgaataaagcctggagcgcctgcagcctgaagcaaggtacccatgaaattacgtcagcggtccagccaggacaatctctgtacggtctgcagctaaccaaccaacagcgaattattatttttggcggcggcctgccagttatttttaatgagcaggtaattggcgccgtcggcgttagcggcggtacggtcgagcaggatcaattattagcccagtgcgccctggattgtttttccgcattataacctgaagcgagaaggtatattatgagctatcgtatgttccgccaggcattctgagtgttaacgaggggaccgtcatgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtatcgggctcagcgcgtccggcgagatagccctcactcatgcccagctgccggcggacgcgccgctggctaccggacacgtcaccgatagcgacgatcaactgcgtacgctcggcgccaacgccgggcagctggttaaagtcctgccgttaagtgagagaaactgaatgtatcgtatctatacccgcaccggggataaaggcaccaccgccctgtacggcggcagccgcatcgagaaagaccatattcgcgtcgaggcctacggcaccgtcgatgaactgatatcccagctgggcgtctgctacgccacgacccgcgacgccgggctgcgggaaagcctgcaccatattcagcagacgctgttcgtgctgggggctgaactggccagcgatgcgcggggcctgacccgcctgagccagacgatcggcgaagaggagatcaccgccctggagcggcttatcgaccgcaatatggccgagagcggcccgttaaaacagttcgtgatcccggggaggaatctcgcctctgcccagctgcacgtggcgcgcacccagtcccgtcggctcgaacgcctgctgacggccatggaccgcgcgcatccgctgcgcgacgcgctcaaacgctacagcaatcgcctgtcggatgccctgttctccatggcgcgaatcgaagagactaggcctgatgcttgcgcttgaactggcctagcaaacacagaaaaaagcccgcacctgacagtgcgggctttttttttcctaggcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccaagcttcaccttttgagccgatgaacaatgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgaactggacggcaaacgccgggaccagtttgacatgatcgaccgatttatcgccgattacgcgatcaacgttgagcgcacagagcaggcaatgcgcctggaggcggtggaaatagcccgtatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctacgcgccgtttaacgccctggcgctgttggtcggttcgcagtgcggccgccccggcgtgttgacgcagtgctcggtggaagaggccaccgagctggagctgggcatgcgtggcttaaccagctacgccgagacggtgtcggtctacggcaccgaagcggtatttaccgacggcgatgatacgccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattactaaaggcgccggggttcagggactgcaaaacggcgcggtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacatcctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcggcagttcgaggtggtgagtgcggtcaacgacatcaatgactatcaggggccgggcaccggctatcgcatctctgccgaacgctgggcggagatcaaaaatattccgggcgtggttcagcccgacaccattgaataaggcggtattcctgtgcaacagacaacccaaattcagccctcttttaccctgaaaacccgcgagggcggggtagcttctgccgatgaacgcgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgctgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacatcgacttagtaagggagtgaccatgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaagcggaggtcagcatgccgttaatagccgggattgatatcggcaacgccaccaccgaggtggcgctggcgtccgactacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttccgcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaaacgggcgctcgcgccagcctctaggtacaaataaaaaaggcacgtcagatgacgtgccttttttcttgtctagcgtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgactagtaaggaggacaattccatggctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattagatttattggatccaggaaacagactagaattatgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaacccgggctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactccagctggatcgggcgctagagtatacatttaaatggtaccctctagtcaaggccttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagct

SEQ ID NO: 75 - DNA, 13402 bp, Artificial Sequence
  Plamid
tagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggggaattaattcccacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcgggaattcatttaaatagtcaaaagcctccgaccggaggcttttgactgctaggcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccaagcttgcgggagagaatgatgaacaagagccaacaagttcagacaatcaccctggccgccgcccagcaaatggcggcggcggtggaaaaaaaagccactgagatcaacgtggcggtggtgttttccgtagttgaccgcggaggcaacacgctgcttatccagcggatggacgaggccttcgtctccagctgcgatatttccctgaataaagcctggagcgcctgcagcctgaagcaaggtacccatgaaattacgtcagcggtccagccaggacaatctctgtacggtctgcagctaaccaaccaacagcgaattattatttttggcggcggcctgccagttatttttaatgagcaggtaattggcgccgtcggcgttagcggcggtacggtcgagcaggatcaattattagcccagtgcgccctggattgtttttccgcattataacctgaagcgagaaggtatattatgagctatcgtatgttccgccaggcattctgagtgttaacgaggggaccgtcatgtcgctttcaccgccaggcgtacgcctgttttacgatccgcgcgggcaccatgccggcgccatcaatgagctgtgctgggggctggaggagcagggggtcccctgccagaccataacctatgacggaggcggtgacgccgctgcgctgggcgccctggcggccagaagctcgcccctgcgggtgggtatcgggctcagcgcgtccggcgagatagccctcactcatgcccagctgccggcggacgcgccgctggctaccggacacgtcaccgatagcgacgatcaactgcgtacgctcggcgccaacgccgggcagctggttaaagtcctgccgttaagtgagagaaactgaatgtatcgtatctatacccgcaccggggataaaggcaccaccgccctgtacggcggcagccgcatcgagaaagaccatattcgcgtcgaggcctacggcaccgtcgatgaactgatatcccagctgggcgtctgctacgccacgacccgcgacgccgggctgcgggaaagcctgcaccatattcagcagacgctgttcgtgctgggggctgaactggccagcgatgcgcggggcctgacccgcctgagccagacgatcggcgaagaggagatcaccgccctggagcggcttatcgaccgcaatatggccgagagcggcccgttaaaacagttcgtgatcccggggaggaatctcgcctctgcccagctgcaccctgatgcttgcgcttgaactggcctagcaaacacagaaaaaagcccgcacctgacagtgcgggctttttttttcctaggcgatctgtgctgtttgccacggtatgcagcaccagcgcgagattatgggctcgcacgctcgactgtcggacgggggcactggaacgagaagtcaggcgagccgtcacgcccttgacaatgccacatcctgagcaaataattcaaccactaaacaaatcaaccgcgtttcccggaggtaaccaagcttcaccttttgagccgatgaacaatgaaaagatcaaaacgatttgcagtactggcccagcgccccgtcaatcaggacgggctgattggcgagtggcctgaagaggggctgatcgccatggacagcccctttgacccggtctcttcagtaaaagtggacaacggtctgatcgtcgaactggacggcaaacgccgggaccagtttgacatgatcgaccgatttatcgccgattacgcgatcaacgttgagcgcacagagcaggcaatgcgcctggaggcggtggaaatagcccgtatgctggtggatattcacgtcagccgggaggagatcattgccatcactaccgccatcacgccggccaaagcggtcgaggtgatggcgcagatgaacgtggtggagatgatgatggcgctgcagaagatgcgtgcccgccggaccccctccaaccagtgccacgtcaccaatctcaaagataatccggtgcagattgccgctgacgccgccgaggccgggatccgcggcttctcagaacaggagaccacggtcggtatcgcgcgctacgcgccgtttaacgccctggcgctgttggtcggttcgcagtgcggccgccccggcgtgttgacgcagtgctcggtggaagaggccaccgagctggagctgggcatgcgtggcttaaccagctacgccgagacggtgtcggtctacggcaccgaagcggtatttaccgacggcgatgatacgccgtggtcaaaggcgttcctcgcctcggcctacgcctcccgcgggttgaaaatgcgctacacctccggcaccggatccgaagcgctgatgggctattcggagagcaagtcgatgctctacctcgaatcgcgctgcatcttcattactaaaggcgccggggttcagggactgcaaaacggcgcggtgagctgtatcggcatgaccggcgctgtgccgtcgggcattcgggcggtgctggcggaaaacctgatcgcctctatgctcgacctcgaagtggcgtccgccaacgaccagactttctcccactcggatattcgccgcaccgcgcgcaccctgatgcagatgctgccgggcaccgactttattttctccggctacagcgcggtgccgaactacgacaacatgttcgccggctcgaacttcgatgcggaagattttgatgattacaacatcctgcagcgtgacctgatggttgacggcggcctgcgtccggtgaccgaggcggaaaccattgccattcgccagaaagcggcgcgggcgatccaggcggttttccgcgagctggggctgccgccaatcgccgacgaggaggtggaggccgccacctacgcgcacggcagcaacgagatgccgccgcgtaacgtggtggaggatctgagtgcggtggaagagatgatgaagcgcaacatcaccggcctcgatattgtcggcgcgctgagccgcagcggctttgaggatatcgccagcaatattctcaatatgctgcgccagcgggtcaccggcgattacctgcagacctcggccattctcgatcggcagttcgaggtggtgagtgcggtcaacgacatcaatgactatcaggggccgggcaccggctatcgcatctctgccgaacgctgggcggagatcaaaaatattccgggcgtggttcagcccgacaccattgaataaggcggtattcctgtgcaacagacaacccaaattcagccctcttttaccctgaaaacccgcgagggcggggtagcttctgccgatgaacgcgccgatgaagtggtgatcggcgtcggccctgccttcgataaacaccagcatcacactctgatcgatatgccccatggcgcgatcctcaaagagctgattgccggggtggaagaagaggggcttcacgcccgggtggtgcgcattctgcgcacgtccgacgtctcctttatggcctgggatgcggccaacctgagcggctcggggatcggcatcggtatccagtcgaaggggaccacggtcatccatcagcgcgatctgctgccgctcagcaacctggagctgttctcccaggcgccgctgctgacgctggagacctaccggcagattggcaaaaacgctgcgcgctatgcgcgcaaagagtcaccttcgccggtgccggtggtgaacgatcagatggtgcggccgaaatttatggccaaagccgcgctatttcatatcaaagagaccaaacatgtggtgcaggacgccgagcccgtcaccctgcacatcgacttagtaagggagtgaccatgagcgagaaaaccatgcgcgtgcaggattatccgttagccacccgctgcccggagcatatcctgacgcctaccggcaaaccattgaccgatattaccctcgagaaggtgctctctggcgaggtgggcccgcaggatgtgcggatctcccgccagacccttgagtaccaggcgcagattgccgagcagatgcagcgccatgcggtggcgcgcaatttccgccgcgcggcggagcttatcgccattcctgacgagcgcattctggctatctataacgcgctgcgcccgttccgctcctcgcaggcggagctgctggcgatcgccgacgagctggagcacacctggcatgcgacagtgaatgccgcctttgtccgggagtcggcggaagtgtatcagcagcggcataagctgcgtaaaggaagctaagcggaggtcagcatgccgttaatagccgggattgatatcggcaacgccaccaccgaggtggcgctggcgtccgactacccgcaggcgagggcgtttgttgccagcgggatcgtcgcgacgacgggcatgaaagggacgcgggacaatatcgccgggaccctcgccgcgctggagcaggccctggcgaaaacaccgtggtcgatgagcgatgtctctcgcatctatcttaacgaagccgcgccggtgattggcgatgtggcgatggagaccatcaccgagaccattatcaccgaatcgaccatgatcggtcataacccgcagacgccgggcggggtgggcgttggcgtggggacgactatcgccctcgggcggctggcgacgctgccggcggcgcagtatgccgaggggtggatcgtactgattgacgacgccgtcgatttccttgacgccgtgtggtggctcaatgaggcgctcgaccgggggatcaacgtggtggcggcgatcctcaaaaaggacgacggcgtgctggtgaacaaccgcctgcgtaaaaccctgccggtggtggatgaagtgacgctgctggagcaggtccccgagggggtaatggcggcggtggaagtggccgcgccgggccaggtggtgcggatcctgtcgaatccctacgggatcgccaccttcttcgggctaagcccggaagagacccaggccatcgtccccatcgcccgcgccctgattggcaaccgttccgcggtggtgctcaagaccccgcagggggatgtgcagtcgcgggtgatcccggcgggcaacctctacattagcggcgaaaagcgccgcggagaggccgatgtcgccgagggcgcggaagccatcatgcaggcgatgagcgcctgcgctccggtacgcgacatccgcggcgaaccgggcacccacgccggcggcatgcttgagcgggtgcgcaaggtaatggcgtccctgaccggccatgagatgagcgcgatatacatccaggatctgctggcggtggatacgtttattccgcgcaaggtgcagggcgggatggccggcgagtgcgccatggagaatgccgtcgggatggcggcgatggtgaaagcggatcgtctgcaaatgcaggttatcgcccgcgaactgagcgcccgactgcagaccgaggtggtggtgggcggcgtggaggccaacatggccatcgccggggcgttaaccactcccggctgtgcggcgccgctggcgatcctcgacctcggcgccggctcgacggatgcggcgatcgtcaacgcggaggggcagataacggcggtccatctcgccggggcggggaatatggtcagcctgttgattaaaaccgagctgggcctcgaggatctttcgctggcggaagcgataaaaaaatacccgctggccaaagtggaaagcctgttcagtattcgtcacgagaatggcgcggtggagttctttcgggaagccctcagcccggcggtgttcgccaaagtggtgtacatcaaggagggcgaactggtgccgatcgataacgccagcccgctggaaaaaattcgtctcgtgcgccggcaggcgaaagagaaagtgtttgtcaccaactgcctgcgcgcgctgcgccaggtctcacccggcggttccattcgcgatatcgcctttgtggtgctggtgggcggctcatcgctggactttgagatcccgcagcttatcacggaagccttgtcgcactatggcgtggtcgccgggcagggcaatattcggggaacagaagggccgcgcaatgcggtcgccaccgggctgctactggccggtcaggcgaattaaacgggcgctcgcgccagcctctaggtacaaataaaaaaggcacgtcagatgacgtgccttttttcttgtctagcgtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgactagtaaggaggacaattccatggctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattagatttattggatccaggaaacagactagaattatgggattgactactaaacctctatctttgaaagttaacgccgctttgttcgacgtcgacggtaccattatcatctctcaaccagccattgctgcattctggagggatttcggtaaggacaaaccttatttcgatgctgaacacgttatccaagtctcgcatggttggagaacgtttgatgccattgctaagttcgctccagactttgccaatgaagagtatgttaacaaattagaagctgaaattccggtcaagtacggtgaaaaatccattgaagtcccaggtgcagttaagctgtgcaacgctttgaacgctctaccaaaagagaaatgggctgtggcaacttccggtacccgtgatatggcacaaaaatggttcgagcatctgggaatcaggagaccaaagtacttcattaccgctaatgatgtcaaacagggtaagcctcatccagaaccatatctgaagggcaggaatggcttaggatatccgatcaatgagcaagacccttccaaatctaaggtagtagtatttgaagacgctccagcaggtattgccgccggaaaagccgccggttgtaagatcattggtattgccactactttcgacttggacttcctaaaggaaaaaggctgtgacatcattgtcaaaaaccacgaatccatcagagttggcggctacaatgccgaaacagacgaagttgaattcatttttgacgactacttatatgctaaggacgatctgttgaaatggtaacccgggctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactccagctggatcgggcgctagagtatacatttaaatggtaccctctagtcaaggccttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgagct

SEQ ID NO: 76 - DNA, 14443 bp, Artificial Sequence
  Plasmid
ttctgataacaaactagcaacaccagaacagcccgtttgcgggcagcaaaacccgtgggaattaattcccctgctcgcgcaggctgggtgccaagctctcgggtaacatcaaggcccgatccttggagcccttcttacagagatgaaaaacaaaccgcgacgccaggcggcatcgcggtctcagagatatgtttacgtagatcgaagagcaccggtgtttaaacgcccttgacgatgccacatcctgagcaaataattcaaccactaaacaaatcaaccgcgtttcccggaggtaaccgagctcatgatcctgtgttgtggtgaagccctgatcgacatgctgccccggcagacgacgctgggtgaggcgggctttgccccttacgcaggcggagcggtcttcaacacggcaattgcgctggggcgtcttggcgtcccttcagccttttttaccggtctttccgacgacatgatgggcgatatcctgcgggagaccctgcgggccagcaaggtggatttcagctattgcgccaccctgtcgcgccccaccaccattgcgttcgttaagctggttgatggccatgcgacctacgctttttacgacgagaacaccgccggccggatgatcaccgaggccgaacttccggccttgggagcggattgcgaagcgctgcatttcggcgccatcagccttattcccgaaccctgcggcagcacctatgaggcgctgatgacgcgcgagcatgagacccgcgtcatctcgctcgatccgaacattcgtcccggcttcatccagaacaagcagtcgcacatggcccgcatccgccgcatggcggcgatgtctgacatcgtcaagttctcggatgaggacctggcgtggttcggtctggaaggcgacgaggacacgcttgcccgccactggctgcaccacggtgcaaaactcgtcgttgtcacccgtggcgccaagggtgccgtgggttacagcgccaatctcaaggtggaagtggcctccgagcgcgtcgaagtggtcgatacggtcggcgccggcgatacgttcgatgccggcattcttgcttcgctgaaaatgcagggcctgctgaccaaagcgcaggtggcttcgctgagcgaagagcagatcagaaaagctttggcgcttggcgcgaaagccgctgcggtcactgtctcgcgggctggcgcaaatccgcctttcgcgcatgaaatcggtttgtgattaattaaagcacgcagtcaaacaaaaaacccgcgccattgcgcgggtttttttatgcccgaaggcgcgccagcacgcagtcaaacaaaaaacccgcgccattgcgcgggtttttttatgcccgaacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtccgtaatacgactcacttaaggccttgactagagggtaccatttaaatgtatactctagcgcccgatccagctggagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagcttgcatgcctgcagcccgggttaccatttcaacagatcgtccttagcatataagtagtcgtcaaaaatgaattcaacttcgtctgtttcggcattgtagccgccaactctgatggattcgtggtttttgacaatgatgtcacagcctttttcctttaggaagtccaagtcgaaagtagtggcaataccaatgatcttacaaccggcggcttttccggcggcaatacctgctggagcgtcttcaaatactactaccttagatttggaagggtcttgctcattgatcggatatcctaagccattcctgcccttcagatatggttctggatgaggcttaccctgtttgacatcattagcggtaatgaagtactttggtctcctgattcccagatgctcgaaccatttttgtgccatatcacgggtaccggaagttgccacagcccatttctcttttggtagagcgttcaaagcgttgcacagcttaactgcacctgggacttcaatggatttttcaccgtacttgaccggaatttcagcttctaatttgttaacatactcttcattggcaaagtctggagcgaacttagcaatggcatcaaacgttctccaaccatgcgagacttggataacgtgttcagcatcgaaataaggtttgtccttaccgaaatccctccagaatgcagcaatggctggttgagagatgataatggtaccgtcgacgtcgaacaaagcggcgttaactttcaaagatagaggtttagtagtcaatcccataattctagtctgtttcctggatccaataaatctaatcttcatgtagatctaattcttcaatcatgtccggcaggttcttcattgggtagttgttgtaaacgatttggtatacggcttcaaataatgggaagtcttcgacagagccacatgtttccaaccattcgtgaacttctttgcaggtaattaaaccttgagcggattggccattcaacaactccttttcacattcccaggcgtccttaccagaagtagccattagcctagcaaccttgacgtttctaccaccagcgcaggtggtgatcaaatcagcaacaccagcagactcttggtagtatgtttcttctctagattctgggaaaaacatttgaccgaatctgatgatctcacccaaaccgactctttggatggcagcagaagcgttgttaccccagcctagaccttcgacgaaaccacaacctaaggcaacaacgttcttcaaagcaccacagatggagataccagcaacatcttcgatgacactaacgtggaagtaaggtctgtggaacaaggcctttagaaccttatggtcgacgtccttgccctcgcctctgaaatcctttggaatgtggtaagcaactgttgtttcagaccagtgttcttgagcgacttcggtggcaatgttagcaccagatagagcaccacattgaatacctagttcctcagtgatgtaagaggatagcaattggacacctttagcaccaacttcaaaaccctttagacaggagatagctctgacgtgtgaatcaacatgacctttcaattggctacagatacggggcaaaaattgatgtggaatgttgaaaacgatgatgtcgacatccttgactgaatcaatcaagtctggattagcaaccaaattgtcgggtagagtgatgccaggcaagtatttcacgttttgatgtctagtatttatgatttcagtcaatttttcaccattgatctcttcttcgaacacccacatttgtactattggagcgaaaacttctgggtatcccttacaattttcggcaaccaccttggcaatagtagtaccccagttaccagatccaatcacagtaaccttgaaaggcttttcggcagccttcaaagaaacagaagaggaacttctctttctaccagcattcaagtggccggaagttaagtttaatctatcagcagcagcagccatggaattgtcctccttactagtcatggtctgtttcctgtgtgaaattgttatccgctcacaattccacacattatacgagccggatgattaattgtcaacagctcatttcagaatatttgccagaaccgttatgatgtcggcgcaaaaaacattatccagaacgggagtgcgccttgagcgacacgaattatgcagtgatttacgacctgcacagccataccacagcttccgatggctgcctgacgccagaagcattggtgcacgctagacaagaaaaaaggcacgtcatctgacgtgccttttttatttgtacctagaggctggcgcgagcgcccgtttaattcgcctgaccggccagtagcagcccggtggcgaccgcattgcgcggcccttctgttccccgaatattgccctgcccggcgaccacgccatagtgcgacaaggcttccgtgataagctgcgggatctcaaagtccagcgatgagccgcccaccagcaccacaaaggcgatatcgcgaatggaaccgccgggtgagacctggcgcagcgcgcgcaggcagttggtgacaaacactttctctttcgcctgccggcgcacgagacgaattttttccagcgggctggcgttatcgatcggcaccagttcgccctccttgatgtacaccactttggcgaacaccgccgggctgagggcttcccgaaagaactccaccgcgccattctcgtgacgaatactgaacaggctttccactttggccagcgggtatttttttatcgcttccgccagcgaaagatcctcgaggcccagctcggttttaatcaacaggctgaccatattccccgccccggcgagatggaccgccgttatctgcccctccgcgttgacgatcgccgcatccgtcgagccggcgccgaggtcgaggatcgccagcggcgccgcacagccgggagtggttaacgccccggcgatggccatgttggcctccacgccgcccaccaccacctcggtctgcagtcgggcgctcagttcgcgggcgataacctgcatttgcagacgatccgctttcaccatcgccgccatcccgacggcattctccatggcgcactcgccggccatcccgccctgcaccttgcgcggaataaacgtatccaccgccagcagatcctggatgtatatcgcgctcatctcatggccggtcagggacgccattaccttgcgcacccgctcaagcatgccgccggcgtgggtgcccggttcgccgcggatgtcgcgtaccggagcgcaggcgctcatcgcctgcatgatggcttccgcgccctcggcgacatcggcctctccgcggcgcttttcgccgctaatgtagaggttgcccgccgggatcacccgcgactgcacatccccctgcggggtcttgagcaccaccgcggaacggttgccaatcagggcgcgggcgatggggacgatggcctgggtctcttccgggcttagcccgaagaaggtggcgatcccgtagggattcgacaggatccgcaccacctggcccggcgcggccacttccaccgccgccattaccccctcggggacctgctccagcagcgtcacttcatccaccaccggcagggttttacgcaggcggttgttcaccagcacgccgtcgtcctttttgaggatcgccgccaccacgttgatcccccggtcgagcgcctcattgagccaccacacggcgtcaaggaaatcgacggcgtcgtcaatcagtacgatccacccctcggcatactgcgccgccggcagcgtcgccagccgcccgagggcgatagtcgtccccacgccaacgcccaccccgcccggcgtctgcgggttatgaccgatcatggtcgattcggtgataatggtctcggtgatggtctccatcgccacatcgccaatcaccggcgcggcttcgttaagatagatgcgagagacatcgctcatcgaccacggtgttttcgccagggcctgctccagcgcggcgagggtcccggcgatattgtcccgcgtccctttcatgcccgtcgtcgcgacgatcccgctggcaacaaacgccctcgcctgcgggtagtcggacgccagcgccacctcggtggtggcgttgccgatatcaatcccggctattaacggcatgctgacctccgcttagcttcctttacgcagcttatgccgctgctgatacacttccgccgactcccggacaaaggcggcattcactgtcgcatgccaggtgtgctccagctcgtcggcgatcgccagcagctccgcctgcgaggagcggaacgggcgcagcgcgttatagatagccagaatgcgctcgtcaggaatggcgataagctccgccgcgcggcggaaattgcgcgccaccgcatggcgctgcatctgctcggcaatctgcgcctggtactcaagggtctggcgggagatccgcacatcctgcgggcccacctcgccagagagcaccttctcgagggtaatatcggtcaatggtttgccggtaggcgtcaggatatgctccgggcagcgggtggctaacggataatcctgcacgcgcatggttttctcgctcatggtcactcccttactaagtcgatgtgcagggtgacgggctcggcgtcctgcaccacatgtttggtctctttgatatgaaatagcgcggctttggccataaatttcggccgcaccatctgatcgttcaccaccggcaccggcgaaggtgactctttgcgcgcatagcgcgcagcgtttttgccaatctgccggtaggtctccagcgtcagcagcggcgcctgggagaacagctccaggttgctgagcggcagcagatcgcgctgatggatgaccgtggtccccttcgactggataccgatgccgatccccgagccgctcaggttggccgcatcccaggccataaaggagacgtcggacgtgcgcagaatgcgcaccacccgggcgtgaagcccctcttcttccaccccggcaatcagctctttgaggatcgcgccatggggcatatcgatcagagtgtgatgctggtgtttatcgaaggcagggccgacgccgatcaccacttcatcggcgcgttcatcggcagaagctaccccgccctcgcgggttttcagggtaaaagagggctgaatttgggttgtctgttgcacaggaataccgccttgttcaatggtgtcgggctgaaccacgcccggaatatttttgatctccgcccagcgttcggcagagatgcgatagccggtgcccggcccctgatagtcattgatgtcgttgaccgcactcaccacctcgaactgccgatcgaaaatggccgaggtctgcaggtaatcgccggtgacccgctggcgcagcatattgagaatattgctggcgatatcctcaaagccgctgcggctcagcgcgccgacaatatcgaggccggtgatgttgcgcttcatcatctcttccaccgcactcagatcctccaccacgttacgcggcggcatctcgttgctgccgtgcgcgtaggtggcggcctccacctcctcgtcggcgattggcggcagccccagctcgcggaaaaccgcctggatcgcccgcgccgctttctggcgaatggcaatggtttccgcctcggtcaccggacgcaggccgccgtcaaccatcaggtcacgctgcaggatgttgtaatcatcaaaatcttccgcatcgaagttcgagccggcgaacatgttgtcgtagttcggcaccgcgctgtagccggagaaaataaagtcggtgcccggcagcatctgcatcagggtgcgcgcggtgcggcgaatatccgagtgggagaaagtctggtcgttggcggacgccacttcgaggtcgagcatagaggcgatcaggttttccgccagcaccgcccgaatgcccgacggcacagcgccggtcatgccgatacagctcaccgcgccgttttgcagtccctgaaccccggcgcctttagtaatgaagatgcagcgcgattcgaggtagagcatcgacttgctctccgaatagcccatcagcgcttcggatccggtgccggaggtgtagcgcattttcaacccgcgggaggcgtaggccgaggcgaggaacgcctttgaccacggcgtatcatcgccgtcggtaaataccgcttcggtgccgtagaccgacaccgtctcggcgtagctggttaagccacgcatgcccagctccagctcggtggcctcttccaccgagcactgcgtcaacacgccggggcggccgcactgcgaaccgaccaacagcgccagggcgttaaacggcgcgtagcgcgcgataccgaccgtggtctcctgttctgagaagccgcggatcccggcctcggcggcgtcagcggcaatctgcaccggattatctttgagattggtgacgtggcactggttggagggggtccggcgggcacgcatcttctgcagcgccatcatcatctccaccacgttcatctgcgccatcacctcgaccgctttggccggcgtgatggcggtagtgatggcaatgatctcctcccggctgacgtgaatatccaccagcatacgggctatttccaccgcctccaggcgcattgcctgctctgtgcgctcaacgttgatcgcgtaatcggcgataaatcggtcgatcatgtcaaactggtcccggcgtttgccgtccagttcgacgatcagaccgttgtccacttttactgaagagaccgggtcaaaggggctgtccatggcgatcagcccctcttcaggccactcgccaatcagcccgtcctgattgacggggcgctgggccagtactgcaaatcgttttgatcttttcattgttcatcggctcaaaaggtgaagcttggttacctccgggaaacgcggttgatttgtttagtggttgaattatttgctcaggatgtggcattgtcaagggcgtgacggctcgcctgacttctcgttccagtgcccccgtccgacagtcgagcgtgcgagcccataatctcgcgctggtgctgcataccgtggcaaacagcacagatcgcctaggaaaaaaaaagcccgcactgtcaggtgcgggcttttttctgtgtttgctaggccagttcaagcgcaagcatcagggtgcagctgggcagaggcgagattcctccccgggatcacgaactgttttaacgggccgctctcggccatattgcggtcgataagccgctccagggcggtgatctcctcttcgccgatcgtctggctcaggcgggtcaggccccgcgcatcgctggccagttcagcccccagcacgaacagcgtctgctgaatatggtgcaggctttcccgcagcccggcgtcgcgggtcgtggcgtagcagacgcccagctgggatatcagttcatcgacggtgccgtaggcctcgacgcgaatatggtctttctcgatgcggctgccgccgtacagggcggtggtgcctttatccccggtgcgggtatagatacgatacattcagtttctctcacttaacggcaggactttaaccagctgcccggcgttggcgccgagcgtacgcagttgatcgtcgctatcggtgacgtgtccggtagccagcggcgcgtccgccggcagctgggcatgagtgagggctatctcgccggacgcgctgagcccgatacccacccgcaggggcgagcttctggccgccagggcgcccagcgcagcggcgtcaccgcctccgtcataggttatggtctggcaggggaccccctgctcctccagcccccagcacagctcattgatggcgccggcatggtgcccgcgcggatcgtaaaacaggcgtacgcctggcggtgaaagcgacatgacggtcccctcgttaacactcagaatgcctggcggaacatacgatagctcataatataccttctcgcttcaggttataatgcggaaaaacaatccagggcgcactgggctaataattgatcctgctcgaccgtaccgccgctaacgccgacggcgccaattacctgctcattaaaaataactggcaggccgccgccaaaaataataattcgctgttggttggttagctgcagaccgtacagagattgtcctggctggaccgctgacgtaatttcatgggtaccttgcttcaggctgcaggcgctccaggctttattcagggaaatatcgcagctggagacgaaggcctcgtccatccgctggataagcagcgtgttgcctccgcggtcaactacggaaaacaccaccgccacgttgatctcagtggcttttttttccaccgccgccgccatttgctgggcggcggccagggtgattgtctgaacttgttggctcttgttcatcattctctcccgcaagcttggttacctccgggaaacgcggttgatttgtttagtggttgaattatttgctcaggatgtggcattgtcaagggcgtgacggctcgcctgacttctcgttccagtgcccccgtccgacagtcgagcgtgcgagcccataatctcgcgctggtgctgcataccgtggcaaacagcacagatcgcctagcagtcaaaagcctccggtcggaggcttttgactatttaaatgaattcccgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaaccggctgaagccggatctgcga

SEQ ID NO: 77 - DNA, 6944 bp, Artificial Sequence
  Plasmid
aattcgcaggaccgtgatacacgggacaggtcactgaatgacgacaatgtcctggaaatcagcgaaccgcgcatctgaagtacatttgagcgactgtaccagaacatgaatgaggcgtttggattaggcgattattagcagggctaagcattttactattattattttccggttgagggatatagagctatcgacaacaaccggaaaaagtttacgtctatattgctgaaggtacaggcgtttccataactatttgctcgcgttttttactcaagaagaaaatgccaaatagcaacatcaggcagacaatacccgaaattgcgaagaaaactgtctggtagcctgcgtggtcaaagagtatcccagtcggcgttgaaagcagcacaatcccaagcgaactggcaatttgaaaaccaatcagaaagatcgtcgacgacaggcgcttatcaaagtttgccacgctgtatttgaagacggatatgacacaaagtggaacctcaatggcatgtaacaacttcactaatgaaataatccaggggttaacgaacagcgcgcaggaaaggatacgcaacgccataatcacaactccgataagtaatgcattttttggccctacccgattcacaaagaaaggaataatcgccatgcacagcgcttcgagtaccacctggaatgagttgagataaccatacaggcgcgttcctacatcgtgtgattcgaataaacctgaataaaagacaggaaaaagttgttgatcaaaaatgttatagaaagaccacgtccccacaataaatatgacgaaaacccagaagtttcgatccttgaaaactgcgataaaatcctctttttttacccctcccgcatctgccgctacgcactggtgatccttatctttaaaacgcatgttgatcatcataaatacagcgccaaatagcgagaccaaccagaagttgatatggggactgatactaaaaaatatgccggcaaagaacgcgccaatagcatagccaaaagatccccaggcgcgcgctgttccatattcgaaatgaaaatttcgcgccattttttcggtgaagctatcaagcaaaccgcatcccgccagataccccaagccaaaaaatagcgcccccagaattagacctacagaaaaattgctttgcagtaacggttcataaacgtaaatcataaacggtccggtcaagaccaggatgaaactcatacaccagatgagcggtttcttcagaccgagtttatcctgaacgatgccgtagaacatcataaatagaatgctggtaaactggttgaccgaataaagtgtacctaattccgtccctgtcaaccctagatgtcctttcagccaaatagcgtataacgaccaccacagcgaccaggaaataaaaaagagaaatgagtaactggatgcaaaacgatagtacgcatttctgaatggaatattcagtgccataattacctgcctgtcgttaaaaaattcacgtcctatttagagataagagcgacttcgccgtttacttctcactattccagttcttgtcgacatggcagcgctgtcattgcccctttcgccgttactgcaagcgctccgcaacgttgagcgagatcgataattcgtcgcatttctctctcatctgtagataatcccgtagaggacagacctgtgagtaacccggcaacgaacgcatctcccgcccccgtgctatcgacacaattcacagacattccagcaaaatggtgaacttgtcctcgataacagaccaccaccccttctgcacctttagtcaccaacagcatggcgatctcatactcttttgccagggcgcatatatcctgatcgttctgtgtttttccactgataagtcgccattcttcttccgagagcttgacgacatccgccagttgtagcgcctgccgcaaacacaagcggagcaaatgctcgtcttgccatagatcttcacgaatattaggatcgaagctgacaaaacctccggcatgccggatcgccgtcatcgcagtaaatgcgctggtacgcgaaggctcggcagacaacgcaattgaacagagatgtaaccattcgccatgtcgccagcagggcaagtctgtcgtctctaaaaaaagatcggcactggggcggaccataaacgtaaatgaacgttccccttgatcgttcagatcgacaagcaccgtggatgtccggtgccattcatcttgcttcagatacgtgatatcgactccctcagttagcagcgttctttgcattaacgcaccaaaaggatcatcccccacccgacctataaacccacttgttccgcctaatctggcgattcccaccgcaacgttagctggcgcgccgccaggacaaggcagtaggcgcccgtctgattctggcaagagatctacgaccgcatcccctaaaacccatactttggctgacatttttttcccttaaattcatctgagttacgcatagtgataaacctctttttcgcaaaatcgtcatggatttactaaaacatgcatattcgatcacaaaacgtcatagttaacgttaacatttgtgatattcatcgcatttatgaaagtaagggactttatttttataaaagttaacgttaacaattcaccaaatttgcttaaccaggatgattaaaatgacgcaatctcgattgcatgcggcgcaaaacgccctagcaaaacttcatgagcaccggggtaacactttctatccccattttcacctcgcgcctcctgccgggtggatgaacgatccaaacggcctgatctggtttaacgatcgttatcacgcgttttatcaacatcatccgatgagcgaacactgggggccaatgcactggggacatgccaccagcgacgatatgatccactggcagcatgagcctattgcgctagcgccaggagacgataatgacaaagacgggtgtttttcaggtagtgctgtcgatgacaatggtgtcctctcacttatctacaccggacacgtctggctcgatggtgcaggtaatgacgatgcaattcgcgaagtacaatgtctggctaccagtcgggatggtattcatttcgagaaacagggtgtgatcctcactccaccagaaggaatcatgcacttccgcgatcctaaagtgtggcgtgaagccgacacatggtggatggtagtcggggcgaaagatccaggcaacacggggcagatcctgctttatcgcggcagttcgttgcgtgaatggaccttcgatcgcgtactggcccacgctgatgcgggtgaaagctatatgtgggaatgtccggactttttcagccttggcgatcagcattatctgatgttttccccgcagggaatgaatgccgagggatacagttaccgaaatcgctttcaaagtggcgtaatacccggaatgtggtcgccaggacgactttttgcacaatccgggcattttactgaacttgataacgggcatgacttttatgcaccacaaagctttttagcgaaggatggtcggcgtattgttatcggctggatggatatgtgggaatcgccaatgccctcaaaacgtgaaggatgggcaggctgcatgacgctggcgcgcgagctatcagagagcaatggcaaacttctacaacgcccggtacacgaagctgagtcgttacgccagcagcatcaatctgtctctccccgcacaatcagcaataaatatgttttgcaggaaaacgcgcaagcagttgagattcagttgcagtgggcgctgaagaacagtgatgccgaacattacggattacagctcggcactggaatgcggctgtatattgataaccaatctgagcgacttgttttgtggcggtattacccacacgagaatttagacggctaccgtagtattcccctcccgcagcgtgacacgctcgccctaaggatatttatcgatacatcatccgtggaagtatttattaacgacggggaagcggtgatgagtagtcgaatctatccgcagccagaagaacgggaactgtcgctttatgcctcccacggagtggctgtgctgcaacatggagcactctggctactgggttaacataatatcaggtggaacaacggatcaacagcgggcaagggatccacgaagcttcccatggtgacgtcaccggttctagatacctaggtgagctctggtaccctctagtcaaggccttaagtgagtcgtattacggactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcttcgcttggtaataaagcccgcttcggcgggcttttttttgttaactacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttctccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtaatgtgagttagctcactcattaggcaccccaggctttacactttatgcttccggctcgtatgttgtgtggaattgtgagcggataacaatttcacacaggaaacagctatgaccatgattacgccaagctacgtaatacgactcactagtgggcagatcttcgaatgcatcgcgcgcaccgtacgtctcgagg

SEQ ID NO: 78 - DNA, 9317 bp, Artificial Sequence
  Plasmid
tcgaggaattcgcaggaccgtgatacacgggacaggtcactgaatgacgacaatgtcctggaaatcagcgaaccgcgcatctgaagtacatttgagcgactgtaccagaacatgaatgaggcgtttggattaggcgattattagcagggctaagcattttactattattattttccggttgagggatatagagctatcgacaacaaccggaaaaagtttacgtctatattgctgaaggtacaggcgtttccataactatttgctcgcgttttttactcaagaagaaaatgccaaatagcaacatcaggcagacaatacccgaaattgcgaagaaaactgtctggtagcctgcgtggtcaaagagtatcccagtcggcgttgaaagcagcacaatcccaagcgaactggcaatttgaaaaccaatcagaaagatcgtcgacgacaggcgcttatcaaagtttgccacgctgtatttgaagacggatatgacacaaagtggaacctcaatggcatgtaacaacttcactaatgaaataatccaggggttaacgaacagcgcgcaggaaaggatacgcaacgccataatcacaactccgataagtaatgcattttttggccctacccgattcacaaagaaaggaataatcgccatgcacagcgcttcgagtaccacctggaatgagttgagataaccatacaggcgcgttcctacatcgtgtgattcgaataaacctgaataaaagacaggaaaaagttgttgatcaaaaatgttatagaaagaccacgtccccacaataaatatgacgaaaacccagaagtttcgatccttgaaaactgcgataaaatcctctttttttacccctcccgcatctgccgctacgcactggtgatccttatctttaaaacgcatgttgatcatcataaatacagcgccaaatagcgagaccaaccagaagttgatatggggactgatactaaaaaatatgccggcaaagaacgcgccaatagcatagccaaaagatccccaggcgcgcgctgttccatattcgaaatgaaaatttcgcgccattttttcggtgaagctatcaagcaaaccgcatcccgccagataccccaagccaaaaaatagcgcccccagaattagacctacagaaaaattgctttgcagtaacggttcataaacgtaaatcataaacggtccggtcaagaccaggatgaaactcatacaccagatgagcggtttcttcagaccgagtttatcctgaacgatgccgtagaacatcataaatagaatgctggtaaactggttgaccgaataaagtgtacctaattccgtccctgtcaaccctagatgtcctttcagccaaatagcgtataacgaccaccacagcgaccaggaaataaaaaagagaaatgagtaactggatgcaaaacgatagtacgcatttctgaatggaatattcagtgccataattacctgcctgtcgttaaaaaattcacgtcctatttagagataagagcgacttcgccgtttacttctcactattccagttcttgtcgacatggcagcgctgtcattgcccctttcgccgttactgcaagcgctccgcaacgttgagcgagatcgataattcgtcgcatttctctctcatctgtagataatcccgtagaggacagacctgtgagtaacccggcaacgaacgcatctcccgcccccgtgctatcgacacaattcacagacattccagcaaaatggtgaacttgtcctcgataacagaccaccaccccttctgcacctttagtcaccaacagcatggcgatctcatactcttttgccagggcgcatatatcctgatcgttctgtgtttttccactgataagtcgccattcttcttccgagagcttgacgacatccgccagttgtagcgcctgccgcaaacacaagcggagcaaatgctcgtcttgccatagatcttcacgaatattaggatcgaagctgacaaaacctccggcatgccggatcgccgtcatcgcagtaaatgcgctggtacgcgaaggctcggcagacaacgcaattgaacagagatgtaaccattcgccatgtcgccagcagggcaagtctgtcgtctctaaaaaaagatcggcactggggcggaccataaacgtaaatgaacgttccccttgatcgttcagatcgacaagcaccgtggatgtccggtgccattcatcttgcttcagatacgtgatatcgactccctcagttagcagcgttctttgcattaacgcaccaaaaggatcatcccccacccgacctataaacccacttgttccgcctaatctggcgattcccaccgcaacgttagctggcgcgccgccaggacaaggcagtaggcgcccgtctgattctggcaagagatctacgaccgcatcccctaaaacccatactttggctgacatttttttcccttaaattcatctgagttacgcatagtgataaacctctttttcgcaaaatcgtcatggatttactaaaacatgcatattcgatcacaaaacgtcatagttaacgttaacatttgtgatattcatcgcatttatgaaagtaagggactttatttttataaaagttaacgttaacaattcaccaaatttgcttaaccaggatgattaaaatgacgcaatctcgattgcatgcggcgcaaaacgccctagcaaaacttcatgagcaccggggtaacactttctatccccattttcacctcgcgcctcctgccgggtggatgaacgatccaaacggcctgatctggtttaacgatcgttatcacgcgttttatcaacatcatccgatgagcgaacactgggggccaatgcactggggacatgccaccagcgacgatatgatccactggcagcatgagcctattgcgctagcgccaggagacgataatgacaaagacgggtgtttttcaggtagtgctgtcgatgacaatggtgtcctctcacttatctacaccggacacgtctggctcgatggtgcaggtaatgacgatgcaattcgcgaagtacaatgtctggctaccagtcgggatggtattcatttcgagaaacagggtgtgatcctcactccaccagaaggaatcatgcacttccgcgatcctaaagtgtggcgtgaagccgacacatggtggatggtagtcggggcgaaagatccaggcaacacggggcagatcctgctttatcgcggcagttcgttgcgtgaatggaccttcgatcgcgtactggcccacgctgatgcgggtgaaagctatatgtgggaatgtccggactttttcagccttggcgatcagcattatctgatgttttccccgcagggaatgaatgccgagggatacagttaccgaaatcgctttcaaagtggcgtaatacccggaatgtggtcgccaggacgactttttgcacaatccgggcattttactgaacttgataacgggcatgacttttatgcaccacaaagctttttagcgaaggatggtcggcgtattgttatcggctggatggatatgtgggaatcgccaatgccctcaaaacgtgaaggatgggcaggctgcatgacgctggcgcgcgagctatcagagagcaatggcaaacttctacaacgcccggtacacgaagctgagtcgttacgccagcagcatcaatctgtctctccccgcacaatcagcaataaatatgttttgcaggaaaacgcgcaagcagttgagattcagttgcagtgggcgctgaagaacagtgatgccgaacattacggattacagctcggcactggaatgcggctgtatattgataaccaatctgagcgacttgttttgtggcggtattacccacacgagaatttagacggctaccgtagtattcccctcccgcagcgtgacacgctcgccctaaggatatttatcgatacatcatccgtggaagtatttattaacgacggggaagcggtgatgagtagtcgaatctatccgcagccagaagaacgggaactgtcgctttatgcctcccacggagtggctgtgctgcaacatggagcactctggctactgggttaacataatatcaggtggaacaacggatcaacagcgggcaagggatccacgaagcttcccatggtgacgtcaccggtaaaccagcaatagacataagcggctatttaacgaccctgccctgaaccgacgaccgggtcgaatttgctttcgaatttctgccattcatccgcttattatacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagggcctcgtgatacgcctatttttataggttaatgtcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggtctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagcc

SEQ ID NO: 79 - DNA, 9317 bp, Artificial Sequence
  Plasmid
tcgaggaattcgcaggaccgtgatacacgggacaggtcactgaatgacgacaatgtcctggaaatcagcgaaccgcgcatctgaagtacatttgagcgactgtaccagaacatgaatgaggcgtttggattaggcgattattagcagggctaagcattttactattattattttccggttgagggatatagagctatcgacaacaaccggaaaaagtttacgtctatattgctgaaggtacaggcgtttccataactatttgctcgcgttttttactcaagaagaaaatgccaaatagcaacatcaggcagacaatacccgaaattgcgaagaaaactgtctggtagcctgcgtggtcaaagagtatcccagtcggcgttgaaagcagcacaatcccaagcgaactggcaatgtgaaaaccaatcagaaagatcgtcgacgacaggcgcttatcaaagtttgccacgctgtatttgaagacggatatgacacaaagtggaacctcaatggcatgtaacaacttcactaatgaaataatccaggggttaacgaacagcgcgcaggaaaggatacgcaacgccataatcacaactccgataagtaatgcattttttggccctacccgattcacaaagaaaggaataatcgccatgcacagcgcttcgagtaccacctggaatgagttgagataaccatacaggcgcgttcctacatcgtgtgattcgaataaacctgaataaaagacaggaaaaagttgttgatcaaaaatgttatagaaagaccacgtccccacaataaatatgacgaaaacccagaagtttcgatccttgaaaactgcgataaaatcctctttttttacccctcccgcatctgccgctacgcactggtgatccttatctttaaaacgcatgttgatcatcataaatacagcgccaaatagcgagaccaaccagaagttgatatggggactgatactaaaaaatatgccggcaaagaacgcgccaatagcatagccaaaagatccccaggcgcgcgctgttccatattcgaaatgaaaatttcgcgccattttttcggtgaagctatcaagcaaaccgcatcccgccagataccccaagccaaaaaatagcgcccccagaattagacctacagaaaaattgctttgcagtaacggttcataaacgtaaatcataaacggtccggtcaagaccaggatgaaactcatacaccagatgagcggtttcttcagaccgagtttatcctgaacgatgccgtagaacatcataaatagaatgctggtaaactggttgaccgaataaagtgtacctaattccgtccctgtcaaccctagatgtcctttcagccaaatagcgtataacgaccaccacagcgaccaggaaataaaaaagagaaatgagtaactggatgcaaaacgatagtacgcatttctgaatggaatattcagtgccataattacctgcctgtcgttaaaaaattcacgtcctatttagagataagagcgacttcgccgtttacttctcactattccagttcttgtcgacatggcagcgctgtcattgcccctttcgccgttactgcaagcgctccgcaacgttgagcgagatcgataattcgtcgcatttctctctcatctgtagataatcccgtagaggacagacctgtgagtaacccggcaacgaacgcatctcccgcccccgtgctatcgacacaattcacagacattccagcaaaatggtgaacttgtcctcgataacagaccaccaccccttctgcacctttagtcaccaacagcatggcgatctcatactcttttgccagggcgcatatatcctgatcgttctgtgtttttccactgataagtcgccattcttcttccgagagcttgacgacatccgccagttgtagcgcctgccgcaaacacaagcggagcaaatgctcgtcttgccatagatcttcacgaatattaggatcgaagctgacaaaacctccggcatgccggatcgccgtcatcgcagtaaatgcgctggtacgcgaaggctcggcagacaacgcaattgaacagagatgtaaccattcgccatgtcgccagcagggcaagtctgtcgtctctaaaaaaagatcggcactggggcggaccataaacgtaaatgaacgttccccttgatcgttcagatcgacaagcaccgtggatgtccggtgccattcatcttgcttcagatacgtgatatcgactccctcagttagcagcgttctttgcattaacgcaccaaaaggatcatcccccacccgacctataaacccacttgttccgcctaatctggcgattcccaccgcaacgttagctggcgcgccgccaggacaaggcagtaggcgcccgtctgattctggcaagagatctacgaccgcatcccctaaaacccatactttggctgacatttttttcccttaaattcatctgagttacgcatagtgataaacctctttttcgcaaaatcgtcatggatttactaaaacatgcatattcgatcacaaaacgtcatagttaacgttaacatttgtgatattcatcgcatttatgaaagtaagggactttatttttataaaagttaacgttaacaattcaccaaatttgcttaaccaggatgattaaaatgacgcaatctcgattgcatgcggcgcaaaacgccctagcaaaacttcatgagcaccggggtaacactttctatccccattttcacctcgcgcctcctgccgggtggatgaacgatccaaacggcctgatctggtttaacgatcgttatcacgcgttttatcaacatcatccgatgagcgaacactgggggccaatgcactggggacatgccaccagcgacgatatgatccactggcagcatgagcctattgcgctagcgccaggagacgataatgacaaagacgggtgtttttcaggtagtgctgtcgatgacaatggtgtcctctcacttatctacaccggacacgtctggctcgatggtgcaggtaatgacgatgcaattcgcgaagtacaatgtctggctaccagtcgggatggtattcatttcgagaaacagggtgtgatcctcactccaccagaaggaatcatgcacttccgcgatcctaaagtgtggcgtgaagccgacacatggtggatggtagtcggggcgaaagatccaggcaacacggggcagatcctgctttatcgcggcagttcgttgcgtgaatggaccttcgatcgcgtactggcccacgctgatgcgggtgaaagctatatgtgggaatgtccggactttttcagccttggcgatcagcattatctgatgttttccccgcagggaatgaatgccgagggatacagttaccgaaatcgctttcaaagtggcgtaatacccggaatgtggtcgccaggacgactttttgcacaatccgggcattttactgaacttgataacgggcatgacttttatgcaccacaaagctttttagcgaaggatggtcggcgtattgttatcggctggatggatatgtgggaatcgccaatgccctcaaaacgtgaaggatgggcaggctgcatgacgctggcgcgcgagctatcagagagcaatggcaaacttctacaacgcccggtacacgaagctgagtcgttacgccagcagcatcaatctgtctctccccgcacaatcagcaataaatatgttttgcaggaaaacgcgcaagcagttgagattcagttgcagtgggcgctgaagaacagtgatgccgaacattacggattacagctcggcactggaatgcggctgtatattgataaccaatctgagcgacttgttttgtggcggtattacccacacgagaatttagacggctaccgtagtattcccctcccgcagcgtgacacgctcgccctaaggatatttatcgatacatcatccgtggaagtatttattaacgacggggaagcggtgatgagtagtcgaatctatccgcagccagaagaacgggaactgtcgctttatgcctcccacggagtggctgtgctgcaacatggagcactctggctactgggttaacataatatcaggtggaacaacggatcaacagcgggcaagggatccacgaagcttcccatggtgacgtcaccggtaaaccagcaatagacataagcggctatttaacgaccctgccctgaaccgacgaccgggtcgaatttgctttcgaatttctgccattcatccgcttattatacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagggcctcgtgatacgcctatttttataggttaatgtcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggtctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagacctgcaggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagcc

SEQ ID NO: 80 - DNA, 100 bp, Artificial Sequence
  Primer
cgtctacccttgttatacctcacaccgcaaggagacgatcatgaccaataatcccccttcagcacagattaagcccggcggtgtaggctggagctgcttc

SEQ ID NO: 81 - DNA, 100 bp, Artificial Sequence
  Primer
gcatcaggcaatgaatacccaatgcgaccagcttcttatatcagaacagccccaacggtttatccgagtagctcaccagccatatgaatatcctccttag

SEQ ID NO: 82 - DNA, 22 bp, Artificial sequence
  Primer
atgaccaataatcccccttcag

SEQ ID NO: 83 - DNA, 21 bp, Artificial Sequence
  Primer
gcttcttatatcagaacagcc

SEQ ID NO: 84 - DNA, 921 bp, Escherichia coli
atgagcgcaagagtatgggtactcggtgatgcggttgttgatttattacccgaaagccaggggagactactacagtgtcctggcggggcgcctgctaatgttgcagtcggtatcgcaaggctgggggggaaaagtgcctttattggcaaagttggcgatgatcctttcggtcgctttatgtatcagacactgagtacagaaaatgttgatacacattatatgtctcttgatcctcaacaacgcacctcaattgtggctgtaggacttgatgagcaaggagaaagaaactttacctttatggtacgcccaagtgccgatctttttttacaacctggtgaccttcctgcatttgggccgggtgaatggctccatctttgttccattgcgctcagtgcagaaccttcccgaagtaccgcatttctggctatggagaaaatacgtcaggctggcggaaacatcagttttgatcccaatatccgcagcgatctctggcagagtgaagcgctattaaggaaataccttgatcgcgcactttcgctggcgaatatcgctaaattgtccgaagaagagttgctattcatcagtggcgaaagccaggttcagcaaggcgcatattcattagtacaacgttattcgttgactttattgcttattacacaaggaaaaaatggcgtacttgtgtattttcaggggcagtttatccactatcccgccaaacctgtttctgtcgtcgatacgaccggggcaggagatgcttttgtcgctggattacttgcaggtctggctgattctggaataccaacaaataccagacagcttgaacgaatcattgcacaagctcagatttgtggtgctctggcgaccacggctaaaggcgcgataaccgccttaccccgacaacacgatctcccttcacaatag

SEQ ID NO: 85 - protein, 306 aa, Escherichia coli
MSARVWVLGDAVVDLLPESQGRLLQCPGGAPANVAVGIARLGGKSAFIGKVGDDPFGRFMYQTLSTENVDTHYMSLDPQQRTSIVAVGLDEQGERNFTFMVRPSADLFLQPGDLPAFGPGEWLHLCSIALSAEPSRSTAFLAMEKIRQAGGNISFDPNIRSDLWQSEALLRKYLDRALSLANIAKLSEEELLFISGESQVQQGAYSLVQRYSLTLLLITQGKNGVLVYFQGQFIHYPAKPVSVVDTTGAGDAFVAGLLAGLADSGIPTNTRQLERIIAQAQICGALATTAKGAITALPRQHDLPSQ

SEQ ID NO: 86 - DNA, 924 bp, Klebsiella pneumoniae
atgaatggaaaaatctgggtactcggcgatgcggtcgtcgatctcctgcccgatggagagggccgcctgctgcaatgccccggcggcgcgccggccaacgtggcggtcggcgtggcgcggctcggcggtgacagcgggtttatcggccgcgtcggcgacgatcccttcggccgttttatgcgtcacaccctggcgcaggagcaagtggatgtgaactatatgcgcctcgatgcggcgcagcgcacctccacggtggtggtcgatctcgatagccacggggagcgcacctttacctttatggtccgtccgagcgccgacctgttccttcagcccgaggatctcccgccgtttgccgccggtcagtggctgcacgtctgctccatcgctctcagcgcggagccgagccgcagcacgacattcgcggcgatggaggcgataaagcgcgccgggggctatgtcagcttcgaccccaatatccgcagcgacctgtggcaggatccgcaggaccttcgcgactgtctcgaccgggcgctggccctcgccgacgccataaaactttcggaagaggagctggcgtttatcagcggcagcgacgacatcgtcagcggcaccgcccggctgaacgcccgcttccagccgacgctactgctggtgacccagggtaaagcgggggtccaggccgccctgcgcgggcaggttagccacttccctgcccgcccggtggtggccgtcgataccaccggcgccggcgatgcctttgtcgccgggctactcgccggcctcgccgcccacggtatcccggacaacctcgcagccctggctcccgacctcgcgctggcgcaaacctgcggcgccctggccaccaccgccaaaggcgccatgaccgccctgccctacagggacgatcttcagcgctcgctgtga

SEQ ID NO: 87 - protein, 307 aa, Klebsiella pneumoniae
MNGKIWVLGDAVVDLLPDGEGRLLQCPGGAPANVAVGVARLGGDSGFIGRVGDDPFGRFMRHTLAQEQVDVNYMRLDAAQRTSTVVVDLDSHGERTFTFMVRPSADLFLQPEDLPPFAAGQWLHVCSIALSAEPSRSTTFAAMEAIKRAGGYVSFDPNIRSDLWQDPQDLRDCLDRALALADAIKLSEEELAFISGSDDIVSGTARLNARFQPTLLLVTQGKAGVQAALRGQVSHFPARPVVAVDTTGAGDAFVAGLLAGLAAHGIPDNLAALAPDLALAQTCGALATTAKGAMTALPYRDDLQRSL

(19) United States
(12) Patent Application Publication
(10) Pub. No.: US 2011/0136190 A1
(43) Pub. Date: Jun. 9, 2011

(54) RECOMBINANT BACTERIA FOR PRODUCING GLYCEROL AND GLYCEROL-DERIVED PRODUCTS FROM SUCROSE

(75) Inventors: ANDREW C. ELIOT, Wilmington, DE (US); Anthony A. Gatenby, Wilmington, DE (US); Tina K. Van Dyk, Wilmington, DE (US)

(73) Assignee: E. I. DU PONT DE NEMOURS AND COMPANY, Wilmington, DE (US)

(21) Appl. No.: 12/943,334

(22) Filed: Nov. 10, 2010

Related U.S. Application Data
(60) Provisional application No. 61/266,605, filed on Dec. 4, 2009.

(51) Int. Cl. C12N 1/21; C12P 7/20; C12P 7/18; C12P 7/42

(52) U.S. Cl. 435/146; 435/252.3; 435/252.33; 435/159; 435/158

(57) ABSTRACT

Recombinant bacteria capable of producing glycerol and glycerol-derived products from sucrose are described. The recombinant bacteria comprise in their genome or on at least one recombinant construct: a nucleotide sequence encoding a polypeptide having sucrose transporter activity; a nucleotide sequence encoding a polypeptide having fructokinase activity; and a nucleotide sequence encoding a polypeptide having sucrose hydrolase activity. These nucleotide sequences are each operably linked to the same or a different promoter. These recombinant bacteria are capable of metabolizing sucrose to produce glycerol and/or glycerol-derived products such as 1,3-propanediol and 3-hydroxypropionic acid.

### FIELD OF THE INVENTION

The invention relates to the fields of microbiology and molecular biology. More specifically, recombinant bacteria having the ability to produce glycerol and glycerol-derived products using sucrose as a carbon source and methods of utilizing such recombinant bacteria are provided.

### BACKGROUND OF THE INVENTION

Many commercially useful microorganisms use glucose as their main carbohydrate source. However, a disadvantage of the use of glucose by microorganisms developed for production of commercially desirable products is the high cost of glucose. The use of sucrose and mixed feedstocks containing sucrose and other sugars as carbohydrate sources for microbial production systems would be more commercially desirable because these materials are readily available at a lower cost.

A production microorganism can function more efficiently when it can utilize any sucrose present in a mixed feedstock. Therefore, when a production microorganism does not have the ability to utilize sucrose efficiently as a major carbon source, it cannot operate as efficiently. For example, bacterial cells typically show preferential sugar use, with glucose being the most preferred. In artificial media containing mixtures of sugars, glucose is typically metabolized to its entirety ahead of other sugars. Moreover, many bacteria lack the ability to utilize sucrose. For example, less than 50% of Escherichia coli strains have the ability to utilize sucrose. Thus, when a production microorganism cannot utilize sucrose as a carbohydrate source, it is desirable to engineer the microorganism so that it can utilize sucrose.

Recombinant bacteria that have been engineered to utilize sucrose by incorporation of sucrose utilization genes have been reported. For example, Livshits et al. (U.S. Pat. No. 6,960,455) describe the production of amino acids using Escherichia coli strains containing genes encoding a metabolic pathway for sucrose utilization. Additionally, Olson et al. (Appl. Microbiol. Biotechnol. 74:1031-1040, 2007) describe Escherichia coli strains carrying genes responsible for sucrose degradation, which produce L-tyrosine or L-phenylalanine using sucrose as a carbon source. However, there is a need for bacterial strains that are capable of producing glycerol and glycerol-derived products using sucrose as carbon source.

### SUMMARY OF THE INVENTION

In one embodiment, the invention provides a recombinant bacterium comprising in its genome or on at least one recombinant construct:
(a) one or more nucleotide sequences encoding a polypeptide or a polypeptide complex having sucrose transporter activity;
(b) a nucleotide sequence encoding a polypeptide having fructokinase activity; and
(c) a nucleotide sequence encoding a polypeptide having sucrose hydrolase activity;

wherein (a), (b) and (c) are each operably linked to the same or a different promoter, further wherein said recombinant bacterium is capable of metabolizing sucrose to produce a product selected from the group consisting of glycerol, 1,3-propanediol and 3-hydroxypropionic acid.

In a second embodiment, the invention provides a process for making glycerol, 1,3-propanediol and/or 3-hydroxypropionic acid from sucrose comprising:
a) culturing the recombinant bacterium disclosed herein in the presence of sucrose; and
b) optionally, recovering the glycerol, 1,3-propanediol and/or 3-hydroxypropionic acid produced.

### BRIEF SEQUENCE DESCRIPTIONS

The following sequences conform with 37 C.F.R. 1.821 1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a bis), and Section 208 and Annex C of the Administrative Instructions). The symbols and format used for nucleotide and amino acid sequence data comply with the rules set forth in 37 C.F.R. §1.822.

TABLE A
Summary of Gene and Protein SEQ ID Numbers

| | Coding | Encoded |
|---|---|---|
| | Sequence | Protein |
| Gene | SEQ ID NO: | SEQ ID NO: |
| GPD1 from Saccharomyces cerevisiae | 1 | 2 |
| GPD2 from Saccharomyces cerevisiae | 3 | 4 |
| GPP1 from Saccharomyces cerevisiae | 5 | 6 |
| GPP2 from Saccharomyces cerevisiae | 7 | 8 |
| dhaB1 from Klebsiella pneumoniae | 9 | 10 |
| dhaB2 from Klebsiella pneumoniae | 11 | 12 |
| dhaB3 from Klebsiella pneumoniae | 13 | 14 |
| aldB from Escherichia coli | 15 | 16 |
| aldA from Escherichia coli | 17 | 18 |
| aldH from Escherichia coli | 19 | 20 |
| galP from Escherichia coli | 21 | 22 |
| cscB from Escherichia coli EC3132 | 23 | 24 |
| cscB from Escherichia coli ATCC13281 | 25 | 26 |
| cscB from Bifidobacterium lactis | 27 | 28 |
| susT1 from Streptococcus pneumoniae | 29 | 30 |
| strain TIGR4 | | |
| susT2 from Streptococcus pneumoniae | 31 | 32 |
| strain TIGR4 | | |
| susX from Streptococcus pneumoniae | 33 | 34 |
| strain TIGR4 | | |
| malE from Streptococcus mutans | 35 | 36 |
| malF from Streptococcus mutans | 37 | 38 |
| malG from Streptococcus mutans | 39 | 40 |
| malK from Streptococcus mutans | 41 | 42 |
| scrK from Agrobacterium tumefaciens | 43 | 44 |
| scrK from Streptococcus mutans | 45 | 46 |
| scrK From Escherichia coli | 84 | 85 |
| scrK from Klebsiella pneumoniae | 86 | 87 |
| cscK from Escherichia coli | 47 | 48 |
| cscK from Enterococcus faecalis | 49 | 50 |
| HXK1 from Saccharomyces cerevisiae | 51 | 52 |
| HXK2 from Saccharomyces cerevisiae | 53 | 54 |
| cscA from Escherichia coli EC3132 | 55 | 56 |
| cscA from Escherichia coli ATCC13281 | 57 | 58 |
| bfrA from Bifidobacterium lactis strain DSM | 59 | 60 |
| 10140^{T} | | |
| SUC2 from Saccharomyces cerevisiae | 61 | 62 |
| scrB from Corynebacterium glutamicum | 63 | 64 |
| sucrose phosphorylase gene from | 65 | 66 |
| Leuconostoc mesenteroides DSM 20193 | | |
| sucP Bifidobacterium adolescentis DSM | 67 | 68 |
| 20083 | | |
| dhaT from Klebsiella pneumoniae | 69 | 70 |

SEQ ID NO:71 is the nucleotide sequence of the coding region of the dhaX gene from Klebsiella pneumoniae.

SEQ ID NO:72 is the nucleotide sequence of plasmid pSYCO101.

SEQ ID NO:73 is the nucleotide sequence of plasmid pSYCO103.

SEQ ID NO:74 is the nucleotide sequence of plasmid pSYCO106.

SEQ ID NO:75 is the nucleotide sequence of plasmid pSYCO109.

SEQ ID NO:76 is the nucleotide sequence of plasmid pSYCO400/AGRO.

SEQ ID NO:77 is the nucleotide sequence of plasmid pScr1 described in Example 1 herein.

SEQ ID NO:78 is the nucleotide sequence of plasmid pBHRcscBKA described in Example 1 herein.

SEQ ID NO:79 is the nucleotide sequence of plasmid pBHRcscBKAmutB described in Example 1 herein.

SEQ ID NOs:80-83 are the nucleotide sequences of primers used to construct strain TTab described in Examples 2-4 herein.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a cell” includes one or more cells and equivalents thereof known to those skilled in the art, and so forth.

In the context of this disclosure, a number of terms and abbreviations are used. The following definitions are provided.

“Open reading frame” is abbreviated as “ORF”.

“Polymerase chain reaction” is abbreviated as “PCR”.

“American Type Culture Collection” is abbreviated as “ATCC”.

The term “recombinant glycerol-producing bacterium” refers to a bacterium that has been genetically engineered to be capable of producing glycerol and/or glycerol-derived products such as 1,3-propanediol and 3-hydroxypropionic acid.

The term “polypeptide or polypeptide complex having sucrose transporter activity” refers to a polypeptide or polypeptide complex that is capable of mediating the transport of sucrose into microbial cells. Examples of polypeptides having sucrose transporter activity include, but are not limited to, sucrose:H+ symporters. Examples of polypeptide complexes having sucrose transporter activity include, but are not limited to, ABC-type transporters. Sucrose:H+ symporters are encoded by, for example, the cscB gene found in E. coli strains such as EC3132 (Jahreis et al., J. Bacteriol. 184:5307-5316, 2002) or ATCC13281 (Olson et al., Appl. Microbiol. Biotechnol. 74:1031-1040, 2007), and Bifidobacterium lactis strain DSM 10140^{T} (Ehrmann et al., Curr. Microbiol. 46(6):391-397, 2003). An example of an ABC-type transporter with activity towards sucrose is the complex encoded by the genes susT1, susT2 and susX in Streptococcus pneumoniae strain TIGR4 (Iyer and Camilli, Molecular Microbiology 66:1-13, 2007). Polypeptides or polypeptide complexes having sucrose transporter activity may also have activity towards other saccharides. An example is the maltose transporter complex of Streptococcus mutans encoded by maIEFGK (Kilic et al., FEMS Microbiol Lett. 266:218, 2007).

The term “polypeptide having fructokinase activity” refers to a polypeptide that has the ability to catalyze the conversion of D-fructose+ATP to fructose-phosphate+ADP. Typical of fructokinase is EC 2.7.1.4. Enzymes that have some ability to phosphorylate fructose, whether or not this activity is their predominant activity, may be referred to as a fructokinase. Abbreviations used for genes encoding fructokinases and proteins having fructokinase activity include, for example, “Frk”, “scrK”, “cscK”, “FK”, and “KHK”. Fructokinase is encoded by the scrK gene in Agrobacterium tumefaciens and Streptococcus mutans; and by the cscK gene in certain Escherichia coli strains.

The term “polypeptide having sucrose hydrolase activity” refers to a polypeptide that has the ability to catalyze the hydrolysis of sucrose to produce glucose and fructose. Such polypeptides are often referred to as “invertases” or “β-fructofuranosidases”. Typical of these enzymes is EC 3.2.1.26. Examples of genes encoding polypeptides having sucrose hydrolase activity are the cscA gene found in E. coli strains EC3132 (Jahreis et al. supra) or ATCC13281 (Olson et al., supra), the bfrA gene from Bifidobacterium lactis strain DSM 10140^{T}, and the SUC2 gene from Saccharomyces cerevisiae (Carlson and Botstein, Cell 28:145, 1982). A polypeptide having sucrose hydrolase activity may also have sucrose phosphate hydrolase activity. An example of such a peptide is encoded by scrB in Corynebacterium glutamicum (Engels et al., FEMS Microbiol Lett. 289:80-89, 2008). A polypeptide having sucrose hydrolase activity may also have sucrose phosphorylase activity. Typical of such an enzyme is EC 2.4.1.7. Examples of genes encoding sucrose phosphorylases having sucrose hydrolase activity are found in Leuconostoc mesenteroides DSM 20193 (Goedl et al., Journal of Biotechnology 129:77-86, 2007) and Bifidobacterium adolescentis DSM 20083 (van den Broek et al., Appl. Microbiol. Biotechnol. 65:219-227, 2004), among others.

The terms “glycerol derivative” and “glycerol-derived products” are used interchangeably herein and refer to a compound that is synthesized from glycerol or in a pathway that includes glycerol. Examples of such products include 3-hydroxypropionic acid, methylglyoxal, 1,2-propanediol, and 1,3-propanediol.

The term “microbial product” refers to a product that is microbially produced, i.e., the result of a microorganism metabolizing a substance. The product may be naturally produced by the microorganism, or the microorganism may be genetically engineered to produce the product.

The terms “phosphoenolpyruvate-sugar phosphotransferase system”, “PTS system”, and “PTS” are used interchangeably herein and refer to the phosphoenolpyruvate-dependent sugar uptake system.

The terms “phosphocarrier protein HPr” and “PtsH” refer to the phosphocarrier protein encoded by ptsH in E. coli. The terms “phosphoenolpyruvate-protein phosphotransferase” and “Ptsl” refer to the phosphotransferase, EC 2.7.3.9, encoded by ptsl in E. coli. The terms “glucose-specific IIA component”, and “Crr” refer to enzymes designated as EC 2.7.1.69, encoded by crr in E. coli. PtsH, Ptsl, and Crr comprise the PTS system.

The term “PTS minus” refers to a microorganism that does not contain a PTS system in its native state or a microorganism in which the PTS system has been inactivated through the inactivation of a PTS gene.

The terms “glycerol-3-phosphate dehydrogenase” and “G3PDH” refer to a polypeptide responsible for an enzyme activity that catalyzes the conversion of dihydroxyacetone phosphate (DHAP) to glycerol 3-phosphate (G3P). In vivo G3PDH may be NAD- or NADP-dependent. When specifically referring to a cofactor specific glycerol-3-phosphate dehydrogenase, the terms “NAD-dependent glycerol-3-phosphate dehydrogenase” and “NADP-dependent glycerol-3-phosphate dehydrogenase” will be used. As it is generally the case that NAD-dependent and NADP-dependent glycerol-3-phosphate dehydrogenases are able to use NAD and NADP interchangeably (for example by the enzyme encoded by gpsA), the terms NAD-dependent and NADP-dependent glycerol-3-phosphate dehydrogenase will be used interchangeably. The NAD-dependent enzyme (EC 1.1.1.8) is encoded, for example, by several genes including GPD1, also referred to herein as DAR1 (coding sequence set forth in SEQ ID NO:1; encoded protein sequence set forth in SEQ ID NO:2), or GPD2 (coding sequence set forth in SEQ ID NO:3; encoded protein sequence set forth in SEQ ID NO:4), or GPD3. The NADP-dependent enzyme (EC 1.1.1.94) is encoded, for example, by gpsA.

The terms “glycerol 3-phosphatase”, “sn-glycerol 3-phosphatase”, “D,L-glycerol phosphatase”, and “G3P phosphatase” refer to a polypeptide having an enzymatic activity that is capable of catalyzing the conversion of glycerol 3-phosphate and water to glycerol and inorganic phosphate. G3P phosphatase is encoded, for example, by GPP1 (coding sequence set forth in SEQ ID NO:5; encoded protein sequence set forth in SEQ ID NO:6), or GPP2 (coding sequence set forth in SEQ ID NO:7; encoded protein sequence set forth in SEQ ID NO:8).

The term “glycerol dehydratase” or “dehydratase enzyme” refers to a polypeptide having enzyme activity that is capable of catalyzing the conversion of a glycerol molecule to the product, 3-hydroxypropionaldehyde (3-HPA).

For the purposes of the present invention the dehydratase enzymes include a glycerol dehydratase (E.C. 4.2.1.30) and a diol dehydratase (E.C. 4.2.1.28) having preferred substrates of glycerol and 1,2-propanediol, respectively. Genes for dehydratase enzymes have been identified in Klebsiella pneumoniae, Citrobacter freundii, Clostridium pasteurianum, Salmonella typhimurium, Klebsiella oxytoca, and Lactobacillus reuteri, among others. In each case, the dehydratase is composed of three subunits: the large or “α” subunit, the medium or “β” subunit, and the small or “γ” subunit. The genes are also described in, for example, Daniel et al. (FEMS Microbiol. Rev. 22, 553 (1999)) and Toraya and Mori (J. Biol. Chem. 274, 3372 (1999)). Genes encoding the large or “α” (alpha) subunit of glycerol dehydratase include dhaB1 (coding sequence set forth in SEQ ID NO:9, encoded protein sequence set forth in SEQ ID NO:10), gldA and dhaB; genes encoding the medium or “β” (beta) subunit include dhaB2 (coding sequence set forth in SEQ ID NO:11, encoded protein sequence set forth in SEQ ID NO:12), gldB, and dhaC; genes encoding the small or “γ” (gamma) subunit include dhaB3 (coding sequence set forth in SEQ ID NO:13, encoded protein sequence set forth in SEQ ID NO:14), gldC, and dhaE. Other genes encoding the large or “α” subunit of diol dehydratase include pduC and pddA; other genes encoding the medium or “β” subunit include pduD and pddB; and other genes encoding the small or “γ” subunit include pduE and pddC.

Glycerol and diol dehydratases are subject to mechanism-based suicide inactivation by glycerol and some other substrates (Daniel et al., FEMS Microbiol. Rev. 22, 553 (1999)). The term “dehydratase reactivation factor” refers to those proteins responsible for reactivating the dehydratase activity. The terms “dehydratase reactivating activity”, “reactivating the dehydratase activity” and “regenerating the dehydratase activity” are used interchangeably and refer to the phenomenon of converting a dehydratase not capable of catalysis of a reaction to one capable of catalysis of a reaction or to the phenomenon of inhibiting the inactivation of a dehydratase or the phenomenon of extending the useful half-life of the dehydratase enzyme in vivo. Two proteins have been identified as being involved as the dehydratase reactivation factor (see, e.g., U.S. Pat. No. 6,013,494 and references therein; Daniel et al., supra; Toraya and Mori, J. Biol. Chem. 274, 3372 (1999); and Tobimatsu et al., J. Bacteriol. 181, 4110 (1999)). Genes encoding one of the proteins include, for example, orfZ, dhaB4, gdrA, pduG and ddrA. Genes encoding the second of the two proteins include, for example, orfX, orf2b, gdrB, pduH and ddrB.

The terms “1,3-propanediol oxidoreductase”, “1,3-propanediol dehydrogenase” and “DhaT” are used interchangeably herein and refer to the polypeptide(s) having an enzymatic activity that is capable of catalyzing the interconversion of 3-HPA and 1,3-propanediol provided the gene(s) encoding such activity is found to be physically or transcriptionally linked to a dehydratase enzyme in its natural (i.e., wild type) setting; for example, the gene is found within a dha regulon as is the case with dhaT from Klebsiella pneumoniae. Genes encoding a 1,3-propanediol oxidoreductase include, but are not limited to, dhaT from Klebsiella pneumoniae, Citrobacter freundii, and Clostridium pasteurianum. Each of these genes encode a polypeptide belonging to the family of type III alcohol dehydrogenases, which exhibits a conserved iron-binding motif, and has a preference for the NAD⁺/NADH linked interconversion of 3-HPA and 1,3-propanediol (Johnson and Lin, J. Bacteriol. 169, 2050 (1987); Daniel et al., J. Bacteriol. 177, 2151 (1995); and Leurs et al., FEMS Microbiol. Lett. 154, 337 (1997)). Enzymes with similar physical properties have been isolated from Lactobacillus brevis and Lactobacillus buchneri (Veiga da Dunha and Foster, Appl. Environ. Microbiol. 58, 2005 (1992)).

The term “dha regulon” refers to a set of associated polynucleotides or open reading frames encoding polypeptides having various biological activities, including but not limited to a dehydratase activity, a reactivation activity, and a 1,3-propanediol oxidoreductase. Typically a dha regulon comprises the open reading frames dhaR, orfY, dhaT, orfX, orfW, dhaB1, dhaB2, dhaB3, and orfZ as described in U.S. Pat. No. 7,371,558.

The terms “aldehyde dehydrogenase” and “Ald” refer to a polypeptide that catalyzes the conversion of an aldehyde to a carboxylic acid. Aldehyde dehydrogenases may use a redox cofactor such as NAD, NADP, FAD, or PQQ. Typical of aldehyde dehydrogenases is EC 1.2.1.3 (NAD-dependent); EC 1.2.1.4 (NADP-dependent); EC 1.2.99.3 (PQQ-dependent); or EC 1.2.99.7 (FAD-dependent). An example of an NADP-dependent aldehyde dehydrogenase is AldB (SEQ ID NO:16), encoded by the E. coli gene aldB (coding sequence set forth in SEQ ID NO:15). Examples of NAD-dependent aldehyde dehydrogenases include AldA (SEQ ID NO:18), encoded by the E. coli gene aldA (coding sequence set forth in SEQ ID NO:17); and AldH (SEQ ID NO:20), encoded by the E. coli gene aldH (coding sequence set forth in SEQ ID NO:19).

The terms “glucokinase” and “Glk” are used interchangeably herein and refer to a protein that catalyzes the conversion of D-glucose+ATP to glucose 6-phosphate+ADP. Typical of glucokinase is EC 2.7.1.2. Glucokinase is encoded by glk in E. coli.

The terms “phosphoenolpyruvate carboxylase” and “Ppc” are used interchangeably herein and refer to a protein that catalyzes the conversion of phosphoenolpyruvate+H₂O+CO₂ to phosphate+oxaloacetic acid. Typical of phosphoenolpyruvate carboxylase is EC 4.1.1.31. Phosphoenolpyruvate carboxylase is encoded by ppc in E. coli.

The terms “glyceraldehyde-3-phosphate dehydrogenase” and “GapA” are used interchangeably herein and refer to a protein having an enzymatic activity capable of catalyzing the conversion of glyceraldehyde 3-phosphate+phosphate+NAD⁺ to 3-phospho-D-glyceroyl-phosphate+NADH+H⁺. Typical of glyceraldehyde-3-phosphate dehydrogenase is EC 1.2.1.12. Glyceraldehyde-3-phosphate dehydrogenase is encoded by gapA in E. coli.

The terms “aerobic respiration control protein” and “ArcA” are used interchangeably herein and refer to a global regulatory protein. The aerobic respiration control protein is encoded by arcA in E. coli.

The terms “methylglyoxal synthase” and “MgsA” are used interchangeably herein and refer to a protein having an enzymatic activity capable of catalyzing the conversion of dihydroxyacetone phosphate to methylglyoxal+phosphate. Typical of methylglyoxal synthase is EC 4.2.3.3. Methylglyoxal synthase is encoded by mgsA in E. coli.

The terms “phosphogluconate dehydratase” and “Edd” are used interchangeably herein and refer to a protein having an enzymatic activity capable of catalyzing the conversion of 6-phospho-gluconate to 2-keto-3-deoxy-6-phospho-gluconate+H₂O. Typical of phosphogluconate dehydratase is EC 4.2.1.12. Phosphogluconate dehydratase is encoded by edd in E. coli.

The term “YciK” refers to a putative enzyme encoded by yciK which is translationally coupled to btuR, the gene encoding Cob(I) alamin adenosyltransferase in E. coli.

The term “cob(I) alamin adenosyltransferase” refers to an enzyme capable of transferring a deoxyadenosyl moiety from ATP to the reduced corrinoid. Typical of cob(I) alamin adenosyltransferase is EC 2.5.1.17. Cob(I) alamin adenosyltransferase is encoded by the gene “btuR” in E. coli, “cobA” in Salmonella typhimurium, and “cobO” in Pseudomonas denitrificans.

The terms “galactose-proton symporter” and “GalP” are used interchangeably herein and refer to a protein having an enzymatic activity capable of transporting a sugar and a proton from the periplasm to the cytoplasm. D-glucose is a preferred substrate for GalP. Galactose-proton symporter is encoded by galP in Escherichia coli (coding sequence set forth in SEQ ID NO:21, encoded protein sequence set forth in SEQ ID NO:22).

The term “non-specific catalytic activity” refers to the polypeptide(s) having an enzymatic activity capable of catalyzing the interconversion of 3-HPA and 1,3-propanediol and specifically excludes 1,3-propanediol oxidoreductase(s). Typically these enzymes are alcohol dehydrogenases. Such enzymes may utilize cofactors other than NAD⁺/NADH, including but not limited to flavins such as FAD or FMN. A gene for a non-specific alcohol dehydrogenase (yqhD) is found, for example, to be endogenously encoded and functionally expressed within E. coli K-12 strains.

The terms “1.6 long GI promoter”, “1.20 short/long GI Promoter”, and “1.5 long GI promoter” refer to polynucleotides or fragments containing a promoter from the Streptomyces lividans glucose isomerase gene as described in U.S. Pat. No. 7,132,527. These promoter fragments include a mutation which decreases their activities as compared to the wild type Streptomyces lividans glucose isomerase gene promoter.

The terms “function” and “enzyme function” are used interchangeably herein and refer to the catalytic activity of an enzyme in altering the rate at which a specific chemical reaction occurs without itself being consumed by the reaction. It is understood that such an activity may apply to a reaction in equilibrium where the production of either product or substrate may be accomplished under suitable conditions.

The terms “polypeptide” and “protein” are used interchangeably herein.

The terms “carbon substrate” and “carbon source” are used interchangeably herein and refer to a carbon source capable of being metabolized by the recombinant bacteria disclosed herein and, particularly, carbon sources comprising fructose and glucose. The carbon source may further comprise other monosaccharides; disaccharides, such as sucrose; oligosaccharides; or polysaccharides.

The terms “host cell” and “host bacterium” are used interchangeably herein and refer to a bacterium capable of receiving foreign or heterologous genes and capable of expressing those genes to produce an active gene product.

The term “production microorganism” as used herein refers to a microorganism, including, but not limited to, those that are recombinant, used to make a specific product such as 1,3-propanediol, glycerol, 3-hydroxypropionic acid, polyunsaturated fatty acids, and the like.

As used herein, “nucleic acid” means a polynucleotide and includes a single or double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” or “nucleic acid fragment” are used interchangeably herein and refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof.

“Gene” refers to a nucleic acid fragment that expresses a specific protein, and which may refer to the coding region alone or may include regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene that is introduced into the host organism by gene transfer. Foreign genes can comprise genes inserted into a non-native organism, genes introduced into a new location within the native host, or chimeric genes.

The term “native nucleotide sequence” refers to a nucleotide sequence that is normally found in the host microorganism.

The term “non-native nucleotide sequence” refers to a nucleotide sequence that is not normally found in the host microorganism.

The term “native polypeptide” refers to a polypeptide that is normally found in the host microorganism.

The term “non-native polypeptide” refers to a polypeptide that is not normally found in the host microorganism.

The terms “encoding” and “coding” are used interchangeably herein and refer to the process by which a gene, through the mechanisms of transcription and translation, produces an amino acid sequence.

The term “coding sequence” refers to a nucleotide sequence that codes for a specific amino acid sequence.

“Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, enhancers, silencers, 5′ untranslated leader sequence (e.g., between the transcription start site and the translation initiation codon), introns, polyadenylation recognition sequences, RNA processing sites, effector binding sites and stem-loop structures.

The term “expression cassette” refers to a fragment of DNA comprising the coding sequence of a selected gene and regulatory sequences preceding (5′ non-coding sequences) and following (3′ non-coding sequences) the coding sequence that are required for expression of the selected gene product. Thus, an expression cassette is typically composed of: 1) a promoter sequence; 2) a coding sequence (i.e., ORF) and, 3) a 3′ untranslated region (e.g., a terminator) that, in eukaryotes, usually contains a polyadenylation site. The expression cassette(s) is usually included within a vector, to facilitate cloning and transformation. Different organisms, including bacteria, yeast, and fungi, can be transformed with different expression cassettes as long as the correct regulatory sequences are used for each host.

“Transformation” refers to the transfer of a nucleic acid molecule into a host organism, resulting in genetically stable inheritance. The nucleic acid molecule may be a plasmid that replicates autonomously, for example, or it may integrate into the genome of the host organism. Host organisms transformed with the nucleic acid fragments are referred to as “recombinant” or “transformed” organisms or “transformants”. “Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance.

“Codon degeneracy” refers to the nature in the genetic code permitting variation of the nucleotide sequence without effecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

The terms “subfragment that is functionally equivalent” and “functionally equivalent subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. Chimeric genes can be designed for use in suppression by linking a nucleic acid fragment or subfragment thereof, whether or not it encodes an active enzyme, in the sense or antisense orientation relative to a promoter sequence.

The term “conserved domain” or “motif” means a set of amino acids conserved at specific positions along an aligned sequence of evolutionarily related proteins. While amino acids at other positions can vary between homologous proteins, amino acids that are highly conserved at specific positions indicate amino acids that are essential in the structure, the stability, or the activity of a protein.

The terms “substantially similar” and “corresponds substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences. Moreover, the skilled artisan recognizes that substantially similar nucleic acid sequences encompassed by this invention are also defined by their ability to hybridize (under moderately stringent conditions, e.g., 0.5×SSC (standard sodium citrate), 0.1% SDS (sodium dodecyl sulfate), 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences are two nucleotide sequences wherein the complement of one of the nucleotide sequences typically has about at least 80% sequence identity, or 90% sequence identity, up to and including 100% sequence identity (i.e., fully complementary) to the other nucleotide sequence.

The term “stringent conditions” or “stringent hybridization conditions” includes reference to conditions under which a probe will selectively hybridize to its target sequence. Probes are typically single stranded nucleic acid sequences which are complementary to the nucleic acid sequences to be detected. Probes are “hybridizable” to the nucleic acid sequence to be detected. Generally, a probe is less than about 1000 nucleotides in length, optionally less than 500 nucleotides in length.

Hybridization methods are well defined. Typically the probe and sample are mixed under conditions which will permit nucleic acid hybridization. This involves contacting the probe and sample in the presence of an inorganic or organic salt under the proper concentration and temperature conditions. Optionally a chaotropic agent may be added. Nucleic acid hybridization is adaptable to a variety of assay formats. One of the most suitable is the sandwich assay format. A primary component of a sandwich-type assay is a solid support. The solid support has adsorbed to it or covalently coupled to it an immobilized nucleic acid probe that is unlabeled and complementary to one portion of the sequence.

Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the thermal melting point (Tₘ) can be approximated from the equation of Meinkoth et al., Anal. Biochem. 138:267-284 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology-Hybridization with Nucleic Acid Probes, Part I, Chapter 2 “Overview of principles of hybridization and the strategy of nucleic acid probe assays”, Elsevier, N.Y. (1993); and Current Protocols in Molecular Biology, Chapter 2, Ausubel et al., Eds., Greene Publishing and Wiley-Interscience, New York (1995). Hybridization and/or wash conditions can be applied for at least 10, 30, 60, 90, 120, or 240 minutes.

“Sequence identity” or “identity” in the context of nucleic acid or polypeptide sequences refers to the nucleic acid bases or amino acid residues in two sequences that are the same when aligned for maximum correspondence over a specified comparison window.

Thus, “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any integer percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, Wis.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, CABIOS. 5:151-153 (1989); Higgins, D. G. et al., Comput. Appl. Biosci. 8:189-191 (1992)) and found in the MegAlign™ program of the LASERGENE bioinformatics computing suite (DNASTAR Inc., Madison, Wis.). For multiple alignments, the default values correspond to GAP PENALTY=10 and GAP LENGTH PENALTY=10. Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program.

The “Clustal W method of alignment” corresponds to the alignment method labeled Clustal W (described by Higgins and Sharp, supra; Higgins, D. G. et al., supra) and found in the MegAlign™ v6.1 program of the LASERGENE bioinformatics computing suite (DNASTAR Inc., Madison, Wis.). Default parameters for multiple alignment correspond to GAP PENALTY=10, GAP LENGTH PENALTY=0.2, Delay Divergen Seqs(%)=30, DNA Transition Weight=0.5, Protein Weight Matrix=Gonnet Series, DNA Weight Matrix=IUB. After alignment of the sequences using the Clustal W program, it is possible to obtain a “percent identity” by viewing the “sequence distances” table in the same program.

“BLASTN method of alignment” is an algorithm provided by the National Center for Biotechnology Information (NCBI) to compare nucleotide sequences using default parameters.

It is well understood by one skilled in the art that many levels of sequence identity are useful in identifying polypeptides, from other species, wherein such polypeptides have the same or similar function or activity. Useful examples of percent identities include, but are not limited to, 50%, 55%, 60%, 65%, 70%, 75%, 80%, 85%, 90%, or 95%, or any integer percentage from 50% to 100%. Indeed, any integer amino acid identity from 50% to 100% may be useful in describing the present invention, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%. Also, of interest is any full-length or partial complement of this isolated nucleotide fragment.

Thus, the invention encompasses more than the specific exemplary nucleotide sequences disclosed herein. For example, alterations in the gene sequence which reflect the degeneracy of the genetic code are contemplated. Also, it is well known in the art that alterations in a gene which result in the production of a chemically equivalent amino acid at a given site, but do not affect the functional properties of the encoded protein are common. Substitutions are defined for the discussion herein as exchanges within one of the following five groups:
1. Small aliphatic, nonpolar or slightly polar residues: Ala, Ser, Thr (Pro, Gly);
2. Polar, negatively charged residues and their amides: Asp, Asn, Glu, Gln;
3. Polar, positively charged residues: His, Arg, Lys;
4. Large aliphatic, nonpolar residues: Met, Leu, Ile, Val (Cys); and
5. Large aromatic residues: Phe, Tyr, Trp.
   Thus, a codon for the amino acid alanine, a hydrophobic amino acid, may be substituted by a codon encoding another less hydrophobic residue (such as glycine) or a more hydrophobic residue (such as valine, leucine, or isoleucine). Similarly, changes which result in substitution of one negatively charged residue for another (such as aspartic acid for glutamic acid) or one positively charged residue for another (such as lysine for arginine) can also be expected to produce a functionally equivalent product. In many cases, nucleotide changes which result in alteration of the N-terminal and C-terminal portions of the protein molecule would also not be expected to alter the activity of the protein.

Each of the proposed modifications is well within the routine skill in the art, as is determination of retention of biological activity of the encoded products. Moreover, the skilled artisan recognizes that substantially similar sequences encompassed by this invention are also defined by their ability to hybridize under stringent conditions, as defined above.

Preferred substantially similar nucleic acid fragments of the instant invention are those nucleic acid fragments whose nucleotide sequences are at least 70% identical to the nucleotide sequence of the nucleic acid fragments reported herein. More preferred nucleic acid fragments are at least 90% identical to the nucleotide sequence of the nucleic acid fragments reported herein. Most preferred are nucleic acid fragments that are at least 95% identical to the nucleotide sequence of the nucleic acid fragments reported herein.

A “substantial portion” of an amino acid or nucleotide sequence is that portion comprising enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Basic Local Alignment Search Tool; Altschul, S. F., et al., J. Mol. Biol., 215:403-410 (1993)). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene-specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence. The instant specification teaches the complete amino acid and nucleotide sequence encoding particular proteins. The skilled artisan, having the benefit of the sequences as reported herein, may now use all or a substantial portion of the disclosed sequences for purposes known to those skilled in this art.

The term “complementary” describes the relationship between two sequences of nucleotide bases that are capable of Watson-Crick base-pairing when aligned in an anti-parallel orientation. For example, with respect to DNA, adenosine is capable of base-pairing with thymine and cytosine is capable of base-pairing with guanine. Accordingly, the instant invention may make use of isolated nucleic acid molecules that are complementary to the complete sequences as reported in the accompanying Sequence Listing and the specification as well as those substantially similar nucleic acid sequences.

The term “isolated” refers to a polypeptide or nucleotide sequence that is removed from at least one component with which it is naturally associated.

“Promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters that cause a gene to be expressed in most cell types at most times are commonly referred to as “constitutive promoters”.

“3′ non-coding sequences”, “transcription terminator” and “termination sequences” are used interchangeably herein and refer to DNA sequences located downstream of a coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory: Cold Spring Harbor, N.Y. (1989). Transformation methods are well known to those skilled in the art and are described infra.

“PCR” or “polymerase chain reaction” is a technique for the synthesis of large quantities of specific DNA segments and consists of a series of repetitive cycles (Perkin Elmer Cetus Instruments, Norwalk, Conn.). Typically, the double-stranded DNA is heat denatured, the two primers complementary to the 3′ boundaries of the target segment are annealed at low temperature and then extended at an intermediate temperature. One set of these three consecutive steps is referred to as a “cycle”.

A “plasmid” or “vector” is an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing an expression cassette(s) into a cell.

The term “genetically altered” refers to the process of changing hereditary material by genetic engineering, transformation and/or mutation.

The term “recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation, natural transduction, natural transposition) such as those occurring without deliberate human intervention.

The terms “recombinant construct”, “expression construct”, “chimeric construct”, “construct”, and “recombinant DNA construct”, are used interchangeably herein. A recombinant construct comprises an artificial combination of nucleic acid fragments, e.g., regulatory and coding sequences that are not found together in nature. For example, a recombinant construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events may result in different levels and patterns of expression (Jones et al., EMBO J. 4:2411-2418 (1985); De Almeida et al., Mol. Gen. Genetics 218:78-86 (1989)), and thus that multiple events may need be screened in order to obtain lines displaying the desired expression level and pattern. Such screening may be accomplished by Southern analysis of DNA, Northern analysis of mRNA expression, immunoblotting analysis of protein expression, or phenotypic analysis, among others.

The term “expression”, as used herein, refers to the production of a functional end-product (e.g., an mRNA or a protein [either precursor or mature]).

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

The term “homologous” refers to proteins or polypeptides of common evolutionary origin with similar catalytic function. The invention may include bacteria producing homologous proteins via recombinant technology.

Disclosed herein are recombinant bacteria comprising in their genome or on at least one recombinant construct: one or more nucleotide sequences encoding a polypeptide or a polypeptide complex having sucrose transporter activity; a nucleotide sequence encoding a polypeptide having fructokinase activity; and a nucleotide sequence encoding a polypeptide having sucrose hydrolase activity. These nucleotide sequences are each operably linked to the same or a different promoter. These recombinant bacteria are capable of metabolizing sucrose to produce glycerol and/or glycerol-derived products such as 1,3-propanediol and 3-hydroxypropionic acid. Bacterial strains capable of producing glycerol and/or glycerol-derived products are highly engineered strains, as described herein below.

Suitable host bacteria for use in the construction of the recombinant bacteria disclosed herein include, but are not limited to organisms of the genera: Escherichia, Streptococcus, Agrobacterium, Bacillus, Corynebacterium, Lactobacillus, Clostridium, Gluconobacter, Citrobacter, Enterobacter, Klebsiella, Aerobacter, Methylobacter, Salmonella, Streptomyces, and Pseudomonas.

In one embodiment the host bacterium is selected from the genera: Escherichia, Klebsiella, Citrobacter, and Aerobacter.

In another embodiment, the host bacterium is Escherichia coli.

In some embodiments, the host bacterium is PTS minus. In these embodiments, the host bacterium is PTS minus in its native state, or may be rendered PTS minus through inactivation of a PTS gene as described below.

In production microorganisms, it is sometimes desirable to unlink the transport of sugars and the use of phosphoenolpyruvate (PEP) for phosphorylation of the sugars being transported.

The term “down-regulated” refers to reduction in, or abolishment of, the activity of active protein(s), as compared to the activity of the wildtype protein(s). The PTS may be inactivated (resulting in a “PTS minus” organism) by down-regulating expression of one or more of the endogenous genes encoding the proteins required in this type of transport. Down-regulation typically occurs when one or more of these genes has a “disruption”, referring to an insertion, deletion, or targeted mutation within a portion of that gene, that results in either a complete gene knockout such that the gene is deleted from the genome and no protein is translated or a protein has been translated such that it has an insertion, deletion, amino acid substitution or other targeted mutation. The location of the disruption in the protein may be, for example, within the N-terminal portion of the protein or within the C-terminal portion of the protein. The disrupted protein will have impaired activity with respect to the protein that was not disrupted, and can be non-functional. Down-regulation that results in low or lack of expression of the protein, could also result via manipulating the regulatory sequences, transcription and translation factors and/or signal transduction pathways or by use of sense, antisense or RNAi technology, etc.

Sucrose transporter polypeptides or polypeptide complexes are polypeptides or polypeptide complexes that are capable of mediating the transport of sucrose into microbial cells. Sucrose transport polypeptides and polypeptide complexes are known, as described above. Examples of polypeptides having sucrose transporter activity include, but are not limited to, CscB from E. coli wild-type strain EC3132 (set forth in SEQ ID NO:24), encoded by gene cscB (coding sequence set forth in SEQ ID NO:23); CscB from E. coli ATCC13281 (set forth in SEQ ID NO:26), encoded by gene cscB (coding sequence set forth in SEQ ID NO:25); and CscB from Bifidobacterium lactis (set forth in SEQ ID NO:28), encoded by gene cscB (coding sequence set forth in SEQ ID NO:27). Examples of polypeptide complexes having sucrose transporter activity include, but are not limited to, the sucrose ABC-type transporter complex from Streptococcus pneumoniae strain TIGR4 comprising three polypeptide subunits set forth in SEQ ID NOs:30, 32, and 34, encoded by genes susT1 (coding sequence set forth in SEQ ID NO:29), susT2 (coding sequence set forth in SEQ ID NO:31), and susX (coding sequence set forth in SEQ ID NO: 33); and the maltose transporter complex of Streptococcus mutans comprising four polypeptide subunits set forth in SEQ ID NOs:36, 38, 40, and 42, encoded by genes malE (coding sequence set forth in SEQ ID NO:35), malF (coding sequence set forth in SEQ ID NO:37), malG (coding sequence set forth in SEQ ID NO:39), and malK (coding sequence set forth in SEQ ID NO:41), respectively.

In one embodiment, the polypeptide having sucrose transporter activity has at least 95% sequence identity, based on the Clustal V method of alignment, to an amino acid sequence as set forth in SEQ ID NO:24, SEQ ID NO:26, or SEQ ID NO:28.

In another embodiment, the polypeptide complex having sucrose transporter activity comprises: a first subunit having at least 95% sequence identity, based on a Clustal V method of alignment, when compared to an amino acid sequence as set forth in SEQ ID NO:30; a second subunit having at least 95% sequence identity, based on a Clustal V method of alignment, when compared to an amino acid sequence as set forth in SEQ ID NO:32; and a third subunit having at least 95% sequence identity, based on a Clustal V method of alignment, when compared to an amino acid sequence as set forth in SEQ ID NO:34.

In another embodiment, the polypeptide complex having sucrose transporter activity comprises: a first subunit having at least 95% sequence identity, based on a Clustal V method of alignment, when compared to an amino acid sequence as set forth in SEQ ID NO:36; a second subunit having at least 95% sequence identity, based on a Clustal V method of alignment, when compared to an amino acid sequence as set forth in SEQ ID NO:38; a third subunit having at least 95% sequence identity, based on a Clustal V method of alignment, when compared to an amino acid sequence as set forth in SEQ ID NO:40; and a fourth subunit having at least 95% sequence identity, based on a Clustal V method of alignment, when compared to an amino acid sequence as set forth in SEQ ID NO:42.

In another embodiment, the polypeptide having sucrose transporter activity corresponds substantially to the amino acid sequence set forth in SEQ ID NO:26.

Polypeptides having fructokinase activity include fructokinases (designated EC 2.7.1.4) and various hexose kinases having fructose phosphorylating activity (EC 2.7.1.3 and EC 2.7.1.1). Fructose phosphorylating activity may be exhibited by hexokinases and ketohexokinases. Representative genes encoding polypeptides from a variety of microorganisms, which may be used to construct the recombinant bacteria disclosed herein, are listed in Table 1. One skilled in the art will know that proteins that are substantially similar to a protein which is able to phosphorylate fructose (such as encoded by the genes listed in Table 1) may also be used.

TABLE 1
Sequences Encoding Enzymes with Fructokinase Activity

| | | | Nucleo- | |
|---|---|---|---|---|
| | | | tide | Protein |
| | | EC | SEQ ID | SEQ ID |
| Source | Gene Name | Number | NO: | NO: |
| Agrobacterium | scrK (fructokinase) | 2.7.1.4 | 43 | 44 |
| tumefaciens | | | | |
| Streptococcus | scrK (fructokinase) | 2.7.1.4 | 45 | 46 |
| mutans | | | | |
| Escherichia | scrK (fructokinase | 2.7.1.4 | 84 | 85 |
| coli | | | | |
| Klebsiella | scrK (fructokinase | 2.7.1.4 | 86 | 87 |
| pneumoniae | | | | |
| Escherichia | cscK (fructokinase) | 2.7.1.4 | 47 | 48 |
| coli | | | | |
| Enterococcus | cscK (fructokinase) | 2.7.1.4 | 49 | 50 |
| faecalis | | | | |
| Saccharomyces | HXK1 (hexokinase) | 2.7.1.1 | 51 | 52 |
| cerevisiae | | | | |
| Saccharomyces | HXK2 (hexokinase) | 2.7.1.1 | 53 | 54 |
| cerevisiae | | | | |

In one embodiment, the polypeptide having fructokinase activity has at least 95% sequence identity, based on the Clustal V method of alignment, to an amino acid sequence as set forth in SEQ ID NO:44, SEQ ID NO:46, SEQ ID NO:48, SEQ ID NO:50, SEQ ID NO:52, SEQ ID NO:54, SEQ ID NO:85, or SEQ ID NO:87.

In another embodiment, the polypeptide having fructokinase activity corresponds substantially to the sequence set forth in SEQ ID NO:48

Polypeptides having sucrose hydrolase activity have the ability to catalyze the hydrolysis of sucrose to produce fructose and glucose. Polypeptides having sucrose hydrolase activity are known, as described above, and include, but are not limited to CscA from E. coli wild-type strain EC3132 (set forth in SEQ ID NO:56), encoded by gene cscA (coding sequence set forth in SEQ ID NO:55), CscA from E. coli ATCC13821 (set forth in SEQ ID NO:58), encoded by gene cscA (coding sequence set forth in SEQ ID NO:57); BfrA from Bifidobacterium lactis strain DSM 10140^{T} (set forth in SEQ ID NO:60), encoded by gene bfrA (coding sequence set forth in SEQ ID NO:59); Suc2p from Saccharomyces cerevisiae (set forth in SEQ ID NO:62), encoded by gene SUC2 (coding sequence set forth in SEQ ID NO:61); ScrB from Corynebacterium glutamicum (set forth in SEQ ID NO:64), encoded by gene scrB (coding sequence set forth in SEQ ID NO:63); sucrose phosphorylase from Leuconostoc mesenteroides DSM 20193 (set forth in SEQ ID NO:66), coding sequence of encoding gene set forth in SEQ ID NO:65; and sucrose phosphorylase from Bifidobacterium adolescentis DSM 20083 (set forth in SEQ ID NO:68), encoded by gene sucP (coding sequence set forth in SEQ ID NO:67).

In one embodiment, the polypeptide having sucrose hydrolase activity has at least 95% sequence identity, based on the Clustal V method of alignment, to an amino acid sequence as set forth in SEQ ID NO:56, SEQ ID NO:58, SEQ ID NO:60, SEQ ID NO:62, SEQ ID NO:64, SEQ ID NO:66, or SEQ ID NO:68.

In another embodiment, the polypeptide having sucrose hydrolase activity corresponds substantially to the amino acid sequence set forth in SEQ ID NO:58.

The coding sequence of genes encoding polypeptides or polypeptide complexes having sucrose transporter activity, polypeptides having fructokinase activity, and polypeptides having sucrose hydrolase activity may be used to isolate nucleotide sequences encoding homologous polypeptides from the same or other microbial species. For example, homologs of the genes may be identified using sequence analysis software, such as BLASTN, to search publicly available nucleic acid sequence databases. Additionally, the isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g. polymerase chain reaction (PCR), Mullis et al., U.S. Pat. No. 4,683,202; ligase chain reaction (LCR), Tabor, S. et al., Proc. Acad. Sci. USA 82, 1074, 1985); or strand displacement amplification (SDA), Walker, et al., Proc. Natl. Acad. Sci. U.S.A., 89: 392, (1992)). For example, the nucleotide sequence encoding the polypeptides described above may be employed as a hybridization probe for the identification of homologs.

One of ordinary skill in the art will appreciate that genes encoding these polypeptides isolated from other sources may also be used in the recombinant bacteria disclosed herein. Additionally, variations in the nucleotide sequences encoding the polypeptides may be made without affecting the amino acid sequence of the encoded polypeptide due to codon degeneracy, and that amino acid substitutions, deletions or additions that produce a substantially similar protein may be included in the encoded protein.

The nucleotide sequences encoding polypeptides or polypeptide complexes having sucrose transporter activity, polypeptides having fructokinase activity, and polypeptides having sucrose hydrolase activity may be isolated using PCR (see, e.g., U.S. Pat. No. 4,683,202) and primers designed to bound the desired sequence, if this sequence is known. Other methods of gene isolation are well known to one skilled in the art such as by using degenerate primers or heterologous probe hybridization. The nucleotide sequences can also be chemically synthesized or purchased from vendors such as DNA2.0 Inc. (Menlo Park, Calif.). Additionally, the entire csc operon may be isolated from the genomic DNA of E. coli strain ATCC13281, as described in detail in Example 1 herein.

Expression of the polypeptides may be effected using one of many methods known to one skilled in the art. For example, the nucleotide sequences encoding the polypeptides described above may be introduced into the bacterium on at least one multicopy plasmid, or by integrating one or more copies of the coding sequences into the host genome. The nucleotide sequences encoding the polypeptides may be introduced into the host bacterium separately (e.g., on separate plasmids) or in any combination (e.g., on a single plasmid, as described in the Examples herein). If the host bacterium contains a gene encoding one of the polynucleotides, then only the remaining nucleotide sequences need to be introduced into the bacterium. For example, if the host bacterium contains a nucleotide sequence encoding a polypeptide having fructokinase activity, only a nucleotide sequence encoding a polypeptide having sucrose transporter activity and a nucleotide sequence encoding a polypeptide having sucrose hydrolase activity need to be introduced into the bacterium to enable sucrose utilization. The introduced coding regions that are either on a plasmid(s) or in the genome may be expressed from at least one highly active promoter. An integrated coding region may either be introduced as a part of a chimeric gene having its own promoter, or it may be integrated adjacent to a highly active promoter that is endogenous to the genome or in a highly expressed operon. Suitable promoters include, but are not limited to, CYC1, HIS3, GAL1, GAL10, ADH1, PGK, PHO5, GAPDH, ADC1, TRP1, URA3, LEU2, ENO, and lac, ara, tet, trp, IP_{L}, IP_{R}, T7, tac, and trc (useful for expression in Escherichia coli) as well as the amy, apr, npr promoters and various phage promoters useful for expression in Bacillus. The promoter may also be the Streptomyces lividans glucose isomerase promoter or a variant thereof, described by Payne et al. (U.S. Pat. No. 7,132,527).

In one embodiment, the recombinant bacteria disclosed herein are capable of producing glycerol. Biological processes for the preparation of glycerol using carbohydrates or sugars are known in yeasts and in some bacteria, other fungi, and algae. Both bacteria and yeasts produce glycerol by converting glucose or other carbohydrates through the fructose-1,6-bisphosphate pathway in glycolysis. In the method of producing glycerol disclosed herein, host bacteria may be used that naturally produce glycerol. In addition, bacteria may be engineered for production of glycerol and glycerol derivatives. The capacity for glycerol production from a variety of substrates may be provided through the expression of the enzyme activities glycerol-3-phosphate dehydrogenase (G3PDH) and/or glycerol-3-phosphatase as described in U.S. Pat. No. 7,005,291. Genes encoding these proteins that may be used for expressing the enzyme activities in a host bacterium are described in U.S. Pat. No. 7,005,291. Suitable examples of genes encoding polypeptides having glycerol-3-phosphate dehydrogenase activity include, but are not limited to, GPD1 from Saccharomyces cerevisiae (coding sequence set forth in SEQ ID NO:1, encoded protein sequence set forth in SEQ ID NO:2) and GPD2 from Saccharomyces cerevisiae (coding sequence set forth in SEQ ID NO:3, encoded protein sequence set forth in SEQ ID NO:4). Suitable examples of genes encoding polypeptides having glycerol-3-phosphatase activity include, but are not limited to, GPP1 from Saccharomyces cerevisiae (coding sequence set forth in SEQ ID NO:5, encoded protein sequence set forth in SEQ ID NO:6) and GPP2 from Saccharomyces cerevisiae (coding sequence set forth in SEQ ID NO:7, encoded protein sequence set forth in SEQ ID NO:8).

Increased production of glycerol may be attained through reducing expression of target endogenous genes. Down-regulation of endogenous genes encoding glycerol kinase and glycerol dehydrogenase activities further enhance glycerol production as described in U.S. Pat. No. 7,005,291. Increased channeling of carbon to glycerol may be accomplished by reducing the expression of the endogenous gene encoding glyceraldehyde 3-phosphate dehydrogenase, as described in U.S. Pat. No. 7,371,558. Down-regulation may be accomplished by using any method known in the art, for example, the methods described above for down-regulation of genes of the PTS system.

Glycerol provides a substrate for microbial production of useful products. Examples of such products, i.e., glycerol derivatives include, but are not limited to, 3-hydroxypropionic acid, methylglyoxal, 1,2-propanediol, and 1,3-propanediol.

In another embodiment, the recombinant bacteria disclosed herein are capable of producing 1,3-propanediol. The glycerol derivative 1,3-propanediol is a monomer having potential utility in the production of polyester fibers and the manufacture of polyurethanes and cyclic compounds. 1,3-Propanediol can be produced by a single microorganism by bioconversion of a carbon substrate other than glycerol or dihydroxyacetone, as described in U.S. Pat. No. 5,686,276. In this bioconversion, glycerol is produced from the carbon substrate, as described above. Glycerol is converted to the intermediate 3-hydroxypropionaldehyde by a dehydratase enzyme, which can be encoded by the host bacterium or can be introduced into the host by recombination. The dehydratase can be glycerol dehydratase (E.C. 4.2.1.30), diol dehydratase (E.C. 4.2.1.28) or any other enzyme able to catalyze this conversion. A suitable example of genes encoding the “α” (alpha), “β” (beta), and “γ” (gamma) subunits of a glycerol dehydratase include, but are not limited to dhaB1 (coding sequence set forth in SEQ ID NO:9), dhaB2 (coding sequence set forth in SEQ ID NO:11), and dhaB3 (coding sequence set forth in SEQ ID NO:13), respectively, from Klebsiella pneumoniae. The further conversion of 3-hydroxypropionaldehyde to 1,3-propandeiol can be catalyzed by 1,3-propanediol dehydrogenase (E.C. 1.1.1.202) or other alcohol dehydrogenases. A suitable example of a gene encoding a 1,3-propanediol dehydrogenase is dhaT from Klebsiella pneumoniae (coding sequence set forth in SEQ ID NO:69, encoded protein sequence set forth in SEQ ID NO:70).

Bacteria can be recombinantly engineered to provide more efficient production of glycerol and the glycerol derivative 1,3-propanediol. For example, U.S. Pat. No. 7,005,291 discloses transformed microorganisms and a method for production of glycerol and 1,3-propanediol with advantages derived from expressing exogenous activities of one or both of glycerol-3-phosphate dehydrogenase and glycerol-3-phosphate phosphatase while disrupting one or both of endogenous activities glycerol kinase and glycerol dehydrogenase.

U.S. Pat. No. 6,013,494 describes a process for the production of 1,3-propanediol using a single microorganism comprising exogenous glycerol-3-phosphate dehydrogenase, glycerol-3-phosphate phosphatase, dehydratase, and 1,3-propanediol oxidoreductase (e.g., dhaT). U.S. Pat. No. 6,136,576 discloses a method for the production of 1,3-propanediol comprising a recombinant microorganism further comprising a dehydratase and protein X (later identified as being a dehydratase reactivation factor peptide).

U.S. Pat. No. 6,514,733 describes an improvement to the process where a significant increase in titer (grams product per liter) is obtained by virtue of a non-specific catalytic activity (distinguished from 1,3-propanediol oxidoreductase encoded by dhaT) to convert 3-hydroxypropionaldehyde to 1,3-propanediol. Additionally, U.S. Pat. No. 7,132,527 discloses vectors and plasmids useful for the production of 1,3-propanediol.

Increased production of 1,3-propanediol may be achieved by further modifications to a host bacterium, including down-regulating expression of some target genes and up-regulating, expression of other target genes, as described in U.S. Pat. No. 7,371,558. For utilization of glucose as a carbon source in a PTS minus host, expression of glucokinase activity may be increased.

Additional genes whose increased or up-regulated expression increases 1,3-propanediol production include genes encoding:
phosphoenolpyruvate carboxylase typically characterized as EC 4.1.1.31
cob(I)alamin adenosyltransferase, typically characterized as EC 2.5.1.17
non-specific catalytic activity that is sufficient to catalyze the interconversion of 3-HPA and 1,3-propanediol, and specifically excludes 1,3-propanediol oxidoreductase(s), typically these enzymes are alcohol dehydrogenases

Genes whose reduced or down-regulated expression increases 1,3-propanediol production include genes encoding:
aerobic respiration control protein
methylglyoxal synthase
acetate kinase
phosphotransacetylase
aldehyde dehydrogenase A
aldehyde dehydrogenase B
triosephosphate isomerase
phosphogluconate dehydratase

In another embodiment, the recombinant bacteria disclosed herein are capable of producing 3-hydroxypropionic acid. 3-Hydroxypropionic acid has utility for specialty synthesis and can be converted to commercially important intermediates by known art in the chemical industry, e.g., acrylic acid by dehydration, malonic acid by oxidation, esters by esterification reactions with alcohols, and 1,3-propanediol by reduction. 3-Hydroxypropionic acid may be produced biologically from a fermentable carbon source by a single microorganism, as described in copending and commonly owned U.S. Patent Application No. 61/187,476. In one representative biosynthetic pathway, a carbon substrate is converted to 3-hydroxypropionaldehyde, as described above for the production of 1,3-propanediol. The 3-hydroxypropionaldehyde is converted to 3-hydroxypropionic acid by an aldehyde dehydrogenase. Suitable examples of aldehyde dehydrogenases include, but are not limited to, AldB (SEQ ID NO:16), encoded by the E. coli gene aldB (coding sequence set forth in SEQ ID NO:15); AldA (SEQ ID NO:18), encoded by the E. coli gene aldA (coding sequence set forth in SEQ ID NO:17); and AldH (SEQ ID NO:20), encoded by the E. coli gene aldH (coding sequence asset forth in SEQ ID NO:19).

Many of the modifications described above to improve 1,3-propanediol production by a recombinant bacterium can also be made to improve 3-hydroxypropionic acid production. For example, the elimination of glycerol kinase prevents glycerol, formed from G3P by the action of G3P phosphatase, from being re-converted to G3P at the expense of ATP. Also, the elimination of glycerol dehydrogenase (for example, gldA) prevents glycerol, formed from DHAP by the action of NAD-dependent glycerol-3-phosphate dehydrogenase, from being converted to dihydroxyacetone. Mutations can be directed toward a structural gene so as to impair or improve the activity of an enzymatic activity or can be directed toward a regulatory gene, including promoter regions and ribosome binding sites, so as to modulate the expression level of an enzymatic activity.

Up-regulation or down-regulation may be achieved by a variety of methods which are known to those skilled in the art. It is well understood that up-regulation or down-regulation of a gene refers to an alteration in the level of activity present in a cell that is derived from the protein encoded by that gene relative to a control level of activity, for example, by the activity of the protein encoded by the corresponding (or non-altered) wild-type gene.

Specific genes involved in an enzyme pathway may be up-regulated to increase the activity of their encoded function(s). For example, additional copies of selected genes may be introduced into the host cell on multicopy plasmids such as pBR322. Such genes may also be integrated into the chromosome with appropriate regulatory sequences that result in increased activity of their encoded functions. The target genes may be modified so as to be under the control of non-native promoters or altered native promoters. Endogenous promoters can be altered in vivo by mutation, deletion, and/or substitution.

Alternatively, it may be useful to reduce or eliminate the expression of certain genes relative to a given activity level. Methods of down-regulating (disrupting) genes are known to those of skill in the art.

Down-regulation can occur by deletion, insertion, or alteration of coding regions and/or regulatory (promoter) regions. Specific down regulations may be obtained by random mutation followed by screening or selection, or, where the gene sequence is known, by direct intervention by molecular biology methods known to those skilled in the art. A particularly useful, but not exclusive, method to effect down-regulation is to alter promoter strength.

Furthermore, down-regulation of gene expression may be used to either prevent expression of the protein of interest or result in the expression of a protein that is non-functional. This may be accomplished for example, by 1) deleting coding regions and/or regulatory (promoter) regions, 2) inserting exogenous nucleic acid sequences into coding regions and/regulatory (promoter) regions, and 3) altering coding regions and/or regulatory (promoter) regions (for example, by making DNA base pair changes). Specific disruptions may be obtained by random mutation followed by screening or selection, or, in cases where the gene sequences in known, specific disruptions may be obtained by direct intervention using molecular biology methods know to those skilled in the art. A particularly useful method is the deletion of significant amounts of coding regions and/or regulatory (promoter) regions.

Methods of altering recombinant protein expression are known to those skilled in the art, and are discussed in part in Baneyx, Curr. Opin. Biotechnol. (1999) 10:411; Ross, et al., J. Bacteriol. (1998) 180:5375; deHaseth, et al., J. Bacteriol. (1998) 180:3019; Smolke and Keasling, Biotechnol. Bioeng. (2002) 80:762; Swartz, Curr. Opin. Biotech. (2001) 12:195; and Ma, et al., J. Bacteriol. (2002) 184:5733.

Recombinant bacteria containing the necessary changes in gene expression for metabolizing sucrose in the production of microbial products including glycerol and glycerol derivatives, as described above, may be constructed using techniques well known in the art, some of which are exemplified in the Examples herein.

The construction of the recombinant bacteria disclosed herein may be accomplished using a variety of vectors and transformation and expression cassettes suitable for the cloning, transformation and expression of coding regions that confer the ability to utilize sucrose in the production of glycerol and its derivatives in a suitable host microorganism. Suitable vectors are those which are compatible with the bacterium employed. Suitable vectors can be derived, for example, from a bacterium, a virus (such as bacteriophage T7 or a M-13 derived phage), a cosmid, a yeast or a plant. Protocols for obtaining and using such vectors are known to those skilled in the art (Sambrook et al., supra).

Initiation control regions, or promoters, which are useful to drive expression of coding regions for the instant invention in the desired host bacterium are numerous and familiar to those skilled in the art. Virtually any promoter capable of driving expression is suitable for use herein. For example, any of the promoters listed above may be used.

Termination control regions may also be derived from various genes native to the preferred hosts. Optionally, a termination site may be unnecessary; however, it is most preferred if included.

For effective expression of the instant polypeptides, nucleotide sequences encoding the polypeptides are linked operably through initiation codons to selected expression control regions such that expression results in the formation of the appropriate messenger RNA.

Particularly useful in the present invention are the vectors pSYCO101, pSYCO103, pSYCO106, and pSYCO109, described in U.S. Pat. No. 7,371,558, and pSYCO400/AGRO, described in U.S. Pat. No. 7,524,660. The essential elements of these vectors are derived from the dha regulon isolated from Klebsiella pneumoniae and from Saccharomyces cerevisiae. Each vector contains the open reading frames dhaB1, dhaB2, dhaB3, dhaX (coding sequence set forth in SEQ ID NO:71), orfX, DAR1, and GPP2 arranged in three separate operons. The nucleotide sequences of pSYCO101, pSYCO103, pSYCO106, pSYCO109, and pSYCO400/AGRO are set forth in SEQ ID NO:72, SEQ ID NO:73, SEQ ID NO:74, SEQ ID NO:75, and SEQ ID NO:76, respectively. The differences between the vectors are illustrated in the chart below [the prefix “p-” indicates a promoter; the open reading frames contained within each “( )” represent the composition of an operon]:

pSYCO101 (SEQ ID NO:72):
p-trc (Dar1_GPP2) in opposite orientation compared to the other 2 pathway operons,
p-1.6 long GI (dhaB1_dhaB2_dhaB3_dhaX), and
p-1.6 long GI (orfY_orfX_orfW).
   pSYCO103 (SEQ ID NO:73):
p-trc (Dar1_GPP2) same orientation compared to the other 2 pathway operons,
p-1.5 long GI (dhaB1_dhaB2_dhaB3_dhaX), and
p-1.5 long GI (orfY_orfX_orfW).
   pSYCO106 (SEQ ID NO:74):
p-trc (Dar1_GPP2) same orientation compared to the other 2 pathway operons,
p-1.6 long GI (dhaB1_dhaB2_dhaB3_dhaX), and
p-1.6 long GI (orfY_orfX_orfW).
   pSYCO109 (SEQ ID NO:75):
p-trc (Dar1_GPP2) same orientation compared to the other 2 pathway operons,
p-1.6 long GI (dhaB1_dhaB2_dhaB3_dhaX), and
p-1.6 long GI (orfY_orfX).
   pSYCO400/AGRO (SEQ ID NO:76):
p-trc (Dar1_GPP2) same orientation compared to the other 2 pathway operons,
p-1.6 long GI (dhaB1_dhaB2_dhaB3_dhaX), and
p-1.6 long GI (orfY_orfX).
p-1.20 short/long GI (scrK) opposite orientation compared to the pathway operons.

Once suitable expression cassettes are constructed, they are used to transform appropriate host bacteria. Introduction of the cassette containing the coding regions into the host bacterium may be accomplished by known procedures such as by transformation (e.g., using calcium-permeabilized cells, or electroporation) or by transfection using a recombinant phage virus (Sambrook et al., supra). Expression cassettes may be maintained on a stable plasmid in a host cell. In addition, expression cassettes may be integrated into the genome of the host bacterium through homologous or random recombination using vectors and methods well known to those skilled in the art. Site-specific recombination systems may also be used for genomic integration of expression cassettes.

In addition to the cells exemplified, cells having single or multiple mutations specifically designed to enhance the production of microbial products including glycerol and/or its derivatives may also be used. Cells that normally divert a carbon feed stock into non-productive pathways, or that exhibit significant catabolite repression may be mutated to avoid these phenotypic deficiencies.

Methods of creating mutants are common and well known in the art. A summary of some methods is presented in U.S. Pat. No. 7,371,558. Specific methods for creating mutants using radiation or chemical agents are well documented in the art. See, for example, Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol. 36, 227 (1992).

After mutagenesis has occurred, mutants having the desired phenotype may be selected by a variety of methods. Random screening is most common where the mutagenized cells are selected for the ability to produce the desired product or intermediate. Alternatively, selective isolation of mutants can be performed by growing a mutagenized population on selective media where only resistant colonies can develop. Methods of mutant selection are highly developed and well known in the art of industrial microbiology. See, for example, Brock, Supra; DeMancilha et al., Food Chem. 14, 313 (1984).

Fermentation media in the present invention comprise sucrose as a carbon substrate. Other carbon substrates such as glucose and fructose may also be present.

In addition to the carbon substrate, a suitable fermentation medium contains, for example, suitable minerals, salts, cofactors, buffers and other components, known to those skilled in the art, suitable for the growth of the cultures and promotion of the enzymatic pathway necessary for production of glycerol and its derivatives, for example 1,3-propanediol. Particular attention is given to Co(II) salts and/or vitamin B₁₂ or precursors thereof in production of 1,3-propanediol.

Adenosyl-cobalamin (coenzyme B₁₂) is an important cofactor for dehydratase activity. Synthesis of coenzyme B₁₂ is found in prokaryotes, some of which are able to synthesize the compound de novo, for example, Escherichia blattae, Klebsiella species, Citrobacter species, and Clostridium species, while others can perform partial reactions. E. coli, for example, cannot fabricate the corrin ring structure, but is able to catalyze the conversion of cobinamide to corrinoid and can introduce the 5′-deoxyadenosyl group. Thus, it is known in the art that a coenzyme B₁₂ precursor, such as vitamin B₁₂, needs be provided in E. coli fermentations. Vitamin B₁₂ may be added continuously to E. coli fermentations at a constant rate or staged as to coincide with the generation of cell mass, or may be added in single or multiple bolus additions.

Although vitamin B₁₂ is added to the transformed E. coli described herein, it is contemplated that other bacteria, capable of de novo vitamin B₁₂ biosynthesis will also be suitable production cells and the addition of vitamin B₁₂ to these bacteria will be unnecessary.

Typically bacterial cells are grown at 25 to 40° C. in an appropriate medium containing sucrose. Examples of suitable growth media for use herein are common commercially prepared media such as Luria Bertani (LB) broth, Sabouraud Dextrose (SD) broth or Yeast medium (YM) broth. Other defined or synthetic growth media may also be used, and the appropriate medium for growth of the particular bacterium will be known by someone skilled in the art of microbiology or fermentation science. The use of agents known to modulate catabolite repression directly or indirectly, e.g., cyclic adenosine 2′:3′-monophosphate, may also be incorporated into the reaction media. Similarly, the use of agents known to modulate enzymatic activities (e.g., methyl viologen) that lead to enhancement of 1,3-propanediol production may be used in conjunction with or as an alternative to genetic manipulations with 1,3-propanediol production strains.

Suitable pH ranges for the fermentation are between pH 5.0 to pH 9.0, where pH 6.0 to pH 8.0 is typical as the initial condition.

Reactions may be performed under aerobic, anoxic, or anaerobic conditions depending on the requirements of the recombinant bacterium. Fed-batch fermentations may be performed with carbon feed, for example, carbon substrate, limited or excess.

Batch fermentation is a commonly used method. Classical batch fermentation is a closed system where the composition of the medium is set at the beginning of the fermentation and is not subject to artificial alterations during the fermentation. Thus, at the beginning of the fermentation, the medium is inoculated with the desired bacterium and fermentation is permitted to occur adding nothing to the system. Typically, however, “batch” fermentation is batch with respect to the addition of carbon source, and attempts are often made at controlling factors such as pH and oxygen concentration. In batch systems, the metabolite and biomass compositions of the system change constantly up to the time the fermentation is stopped. Within batch cultures, cells moderate through a static lag phase to a high growth log phase and finally to a stationary phase where growth rate is diminished or halted. If untreated, cells in the stationary phase will eventually die. Cells in log phase generally are responsible for the bulk of production of end product or intermediate.

A variation on the standard batch system is the Fed-Batch system. Fed-Batch fermentation processes are also suitable for use herein and comprise a typical batch system with the exception that the substrate is added in increments as the fermentation progresses. Fed-Batch systems are useful when catabolite repression is apt to inhibit the metabolism of the cells and where it is desirable to have limited amounts of substrate in the media. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch fermentations are common and well known in the art and examples may be found in Brock, supra.

Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth.

Continuous fermentation allows for the modulation of one factor or any number of factors that affect cell growth or end product concentration. For example, one method will maintain a limiting nutrient such as the carbon source or nitrogen level at a fixed rate and allow all other parameters to moderate. In other systems, a number of factors affecting growth can be altered continuously while the cell concentration, measured by the turbidity of the medium, is kept constant. Continuous systems strive to maintain steady state growth conditions, and thus the cell loss due to medium being drawn off must be balanced against the cell growth rate in the fermentation. Methods of modulating nutrients and growth factors for continuous fermentation processes as well as techniques for maximizing the rate of product formation are well known in the art of industrial microbiology and a variety of methods are detailed by Brock, supra.

It is contemplated that the present invention may be practiced using batch, fed-batch or continuous processes and that any known mode of fermentation would be suitable. Additionally, it is contemplated that cells may be immobilized on a substrate as whole cell catalysts and subjected to fermentation conditions for production of glycerol and glycerol derivatives, such as 1,3-propanediol.

In one embodiment, a process for making glycerol, 1,3-propanediol, and/or 3-hydroxypropionic acid from sucrose is provided. The process comprises the steps of culturing a recombinant bacterium, as described above, in the presence of sucrose, and optionally recovering the glycerol, 1,3-propanediol, and/or 3-hydroxypropionic acid produced. The product may be recovered using methods known in the art. For example, solids may be removed from the fermentation medium by centrifugation, filtration, decantation, or the like. Then, the product may be isolated from the fermentation medium, which has been treated to remove solids as described above, using methods such as distillation, liquid-liquid extraction, or membrane-based separation.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### General Methods

Standard recombinant DNA and molecular cloning techniques described in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987).

Materials and methods suitable for the maintenance and growth of bacterial cultures are well known in the art. Techniques suitable for use in the following Examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989). All reagents, restriction enzymes and materials described for the growth and maintenance of bacterial cells may be obtained from Aldrich Chemicals (Milwaukee, Wis.), BD Diagnostic Systems (Sparks, Md.), Life Technologies (Rockville, Md.), New England Biolabs (Beverly, Mass.), or Sigma Chemical Company (St. Louis, Mo.).

The meaning of abbreviations is as follows: “s” means second(s), “min” means minute(s), “h” means hour(s), “nm” means nanometers, “μL” means microliter(s), “mL” means milliliter(s), “L” means liter(s), “mM” means millimolar, “M” means molar, “g” means gram(s), “μg” means microgram(s), “bp” means base pair(s), “kbp” means kilobase pair(s), “rpm” means revolutions per minute, “ATCC” means American Type Culture Collection, Manassas, Va., “dH₂O” means distilled water.

### Example 1

### Construction of csc Operon Expression Plasmids

This Example illustrates the construction of csc operon expression plasmids pBHRcscBKA and pBHRcscBKAmutB.

Genomic DNA was isolated from E. coli strain ATCC13281 and digested with EcoRI and BamHI. Fragments approximately 4 kbp in length were isolated by Tris-Borate-EDTA agarose gel electrophoresis and ligated with plasmid vector pLitmus28 (New England Biolabs, Beverly, Mass.) that had also been digested with EcoRI and BamHI. The resulting plasmids were used to transform E. coli strain DH5alpha (Invitrogen, Carlsbad, Calif.), and transformants containing the genes required for sucrose utilization were identified by growth on MacConkey sucrose agar (MacConkey agar base from Difco, Sparks, Md.) containing 100 μg/mL ampicillin. Plasmid DNA was isolated from a colony that had acquired the ability to metabolize sucrose, and the plasmid (designated pScr1; set forth in SEQ ID NO:77) was sequenced to identify the region of DNA necessary for sucrose utilization. The insert was 4140 bp in length and contained putatitve open reading frames homologous to the known E. coli sucrose utilization genes cscB, cscK, and cscA (Jahreis et al., J. Bacteriol. 184:5307-5316, 2002).

The csc operon was subsequently moved to plasmid pBHR1 (MobiTec GmbH, Goettingen, Germany) using the following procedure. Plasmid pScr1 was digested with XhoI and treated with Klenow fragment to yield blunt ends, followed by digestion with Agel. The resulting 4175 bp fragment containing the csc genes was isolated by gel purification. The plasmid pBHR1 was digested with Agel and Nael, and the resulting 5142 bp fragment was isolated by gel purification. The two gel purified fragments were then ligated, and the resulting plasmid was used to transform E. coli strain DH5alpha. Transformants were selected by growth on Luria Bertani (LB) agar containing 50 μg/mL kanamycin. Plasmid DNA was isolated from the transformants, and the sequence of the plasmid was verified. The plasmid was designated pBHRcscBKA (set forth in SEQ ID NO:78).

Another expression plasmid was generated by making a single base pair substitution in pBHRcscBKA using the Stratagene QuikChange® Site-Directed Mutagenesis Kit (Stratagene, La Jolla, Calif.). The thymine base at position 4263 was replaced with guanosine, and the resulting plasmid was designated pBHRcscBKAmutB (set forth in SEQ ID NO:79). This substitution resulted in the replacement of a glutamine residue with histidine in the polypeptide encoded by cscB, a change which was reported to alter the transport capabilities of the homologous protein from E. coli strain EC3132 (Jahreis et al., supra).

### Examples 2-4

### Construction of Recombinant E. coli Strains Comprising the csc Operon

These Examples illustrate the construction of recombinant E. coli strains that were transformed with plasmids comprising the csc operon. The consumption of sucrose and the production of the end products 1,3-propanediol (PDO) and glycerol from sucrose by these recombinant strains were demonstrated.

E. coli strain TTab pSYCO109

Strain TTab was generated by deletion of the aldB gene from strain TT aldA, described in U.S. Pat. No. 7,371,558 (Example 17). Briefly, an aldB deletion was made by first replacing 1.5 kbp of the coding region of aldB in E. coli strain MG1655 (available from The American Type Culture Collection as ATCC No: 700926) with the FRT-CmR-FRT cassette of the pKD3 plasmid (Datsenko and Wanner, Proc. Natl. Acad. Sci. USA 97:6640-6645, 2000). A replacement cassette was amplified with the primer pair SEQ ID NO:80 and SEQ ID NO:81 using pKD3 as the template. The primer SEQ ID NO:80 contains 80 bp of homology to the 5′-end of aldB and 20 bp of homology to pKD3. Primer SEQ ID NO:81 contains 80 bp of homology to the 3′ end of aldB and 20 bp homology to pKD3. The PCR products were gel-purified and electroporated into MG1655/pKD46 competent cells (U.S. Pat. No. 7,371,558). Recombinant strains were selected on LB plates with 12.5 mg/L of chloroamphenicol. The deletion of the aldB gene was confirmed by PCR, using the primer pair SEQ ID NO:82 and SEQ ID NO:83. The wild-type strain gave a 1.5 kbp PCR product while the recombinant strain gave a characteristic 1.1 kbp PCR product. A P1 lysate was prepared and used to move the mutation to the TT aldA strain to form the TT aldAΔaldB::Cm strain. A chloramphenicol-resistant clone was checked by genomic PCR with the primer pair SEQ ID NO:82 and SEQ ID NO:83 to ensure that the mutation was present. The chloramphenicol resistance marker was removed using the FLP recombinase (Datsenko and Wanner, supra) to create strain TTab. Strain TTab was then transformed with pSYCO109 (set forth in SEQ ID NO:75), described in U.S. Pat. No. 7,371,558, to generate strain TTab pSYCO109.

As described in the cited references, strain TTab is a derivative of E. coli strain FM5 (ATCC No. 53911) containing the following modifications:
deletion of glpK, gldA, ptsHl, cm edd, arcA, mgsA, qor, ackA, pta, aldA and aldB genes;
upregulation of galP, glk, btuR, ppc, and yqhD genes; and
downregulation of gapA gene.

Plasmid pSYCO109 contains genes encoding a glycerol production pathway (DAR1 and GPP2) and genes encoding a glycerol dehydratase and associated reactivating factor (dhaB123, dhaX, orfX, orfY).

Strain TTab/pSYCO109 was transformed with each of the two csc operon overexpression plasmids pBHRcscBKA and pBHRcscBKAmutB, described in Example 1. Transformants were selected by growth on LB agar containing 50 μg/mL of spectinomycin and 50 μg/mL of kanamycin. Individual colonies were picked and grown overnight at 34° C. with shaking (250 rpm) in LB broth with the same antibiotics. The control strain TTab/pSYCO109 was grown under identical conditions with the exception of the kanamycin.

These overnight cultures were diluted into TM3 medium containing 10.5 g/L sucrose to an optical density of 0.01 units measured at 550 nm. TM3 is a minimal medium containing 13.6 g/L KH₂PO₄, 2.04 g/L citric acid dihydrate, 2 g/L magnesium sulfate heptahydrate, 0.33 g/L ferric ammonium citrate, 0.5 g/L yeast extract, 3 g/L ammonium sulfate, 0.2 g/L CaCl₂.2H₂O, 0.03 g MnSO₄.H₂O, 0.01 g/L NaCl, 1 mg/L FeSO₄.7H₂O, 1 mg/L, CoCl₂.6H₂O, 1 mg/L ZnSO₄.7H₂O, 0.1 mg/L CuSO₄.5H₂O, 0.1 mg/L H₃BO₄, 0.1 mg/L NaMoO₄.2H₂O and sufficient NH₄OH to provide a final pH of 6.8. Vitamin B₁₂ was added to the medium to a concentration of 0.1 mg/L. The cultures were incubated at 34° C. with shaking (225 rpm) for 24 hours. Aliquots were removed at 0, 5, 8, 11, 14, 17, 20 and 23 hours after inoculation, and the concentrations of sucrose, glycerol and 1,3-propanediol (PDO) in the broth were determined by high performance liquid chromatography.

Chromatographic separation was achieved using an Aminex HPX-87P column (Bio-Rad, Hercules, Calif.) with an isocratic mobile phase of dH₂O at a flow rate of 0.5 mL/min and a column temperature of 60° C. Eluted compounds were quantified by refractive index detection with reference to a standard curve prepared from commercially purchased pure compounds dissolved to known concentrations in the TM3 medium. Retention times were sucrose at 12.2 min, 1,3-propanediol at 17.9 min, and glycerol at 23.6 min.

Both csc expression plasmids (Examples 3 and 4) resulted in metabolism of sucrose and production of PDO and glycerol while the parent control strain (Example 2, Comparative) was unable to metabolize sucrose or produce PDO or glycerol under these conditions (see Tables 2-4). The data points given in the tables represents the average of measurements made on two duplicate cultures.

TABLE 2
Sucrose consumption

| | Sucrose (g/L) | | |
|---|---|---|---|
| | Example 2, | | |
| Time | Comparative | Example 3 | Example 4 |
| (h) | Control Strain | +pBHRcscBKA | +pBHRcscBKAmutB |
| 0 | 10.48 | 10.48 | 10.48 |
| 6 | 10.14 | 10.05 | 10.08 |
| 12 | 10.34 | 9.87 | 10.17 |
| 18 | 10.28 | 7.31 | 10.17 |
| 24 | 10.32 | 0.65 | 10.13 |
| 30 | 10.37 | 0.00 | 8.44 |
| 36 | 10.36 | 0.00 | 3.32 |
| 42 | 10.33 | 0.00 | 0.00 |

TABLE 3
PDO Production

| | PDO (g/L) | | |
|---|---|---|---|
| | Example 2, | | |
| Time | Comparative | Example 3 | Example 4 |
| (h) | Control Strain | +pBHRcscBKA | +pBHRcscBKAmutB |
| 0 | 0.00 | 0.00 | 0.00 |
| 6 | 0.00 | 0.00 | 0.00 |
| 12 | 0.00 | 0.00 | 0.00 |
| 18 | 0.00 | 0.41 | 0.00 |
| 24 | 0.00 | 2.20 | 0.02 |
| 30 | 0.00 | 3.15 | 0.24 |
| 36 | 0.00 | 3.15 | 1.35 |
| 42 | 0.00 | 3.06 | 2.82 |

TABLE 4
Glycerol production

| | Glycerol (g/L) | | |
|---|---|---|---|
| | Example 2, | | |
| Time | Comparative | Example 3 | Example 4 |
| (h) | Control Strain | +pBHRcscBKA | +pBHRcscBKAmutB |
| 0 | 0.00 | 0.00 | 0.00 |
| 6 | 0.00 | 0.00 | 0.00 |
| 12 | 0.00 | 0.08 | 0.00 |
| 18 | 0.00 | 0.96 | 0.00 |
| 24 | 0.00 | 3.24 | 0.00 |
| 30 | 0.00 | 2.54 | 0.60 |
| 36 | 0.00 | 2.52 | 2.33 |
| 42 | 0.00 | 2.49 | 2.98 |

What is claimed is:

1. A recombinant bacterium comprising in its genome or on at least one recombinant construct:
(a) one or more nucleotide sequences encoding a polypeptide or a polypeptide complex having sucrose transporter activity;
(b) a nucleotide sequence encoding a polypeptide having fructokinase activity; and
(c) a nucleotide sequence encoding a polypeptide having sucrose hydrolase activity;
wherein (a), (b) and (c) are each operably linked to the same or a different promoter,
further wherein said recombinant bacterium is capable of metabolizing sucrose to produce a product selected from the group consisting of glycerol, 1,3-propanediol and 3-hydroxypropionic acid.

2. The recombinant bacterium of claim 1 wherein the polypeptide having sucrose transporter activity has at least 95% sequence identity, based on a Clustal V method of alignment, when compared to an amino acid sequence as set forth in SEQ ID NO:24, SEQ ID NO:26, or SEQ ID NO:28.

3. The recombinant bacterium of claim 1 wherein the polypeptide complex having sucrose transporter activity comprises:
a) a first subunit having at least 95% sequence identity, based on a Clustal V method of alignment, when compared to an amino acid sequence as set forth in SEQ ID NO:30;
b) a second subunit having at least 95% sequence identity, based on a Clustal V method of alignment, when compared to an amino acid sequence as set forth in SEQ ID NO:32; and
c) a third subunit having at least 95% sequence identity, based on a Clustal V method of alignment, when compared to an amino acid sequence as set forth in SEQ ID NO:34.

4. The recombinant bacterium of claim 1 wherein the polypeptide complex having sucrose transporter activity comprises:
a) a first subunit having at least 95% sequence identity, based on a Clustal V method of alignment, when compared to an amino acid sequence as set forth in SEQ ID NO:36;
b) a second subunit having at least 95% sequence identity, based on a Clustal V method of alignment, when compared to an amino acid sequence as set forth in SEQ ID NO:38;
c) a third subunit having at least 95% sequence identity, based on a Clustal V method of alignment, when compared to an amino acid sequence as set forth in SEQ ID NO:40; and
d) a fourth subunit having at least 95% sequence identity, based on a Clustal V method of alignment, when compared to an amino acid sequence as set forth in SEQ ID NO:42.

5. The recombinant bacterium of claim 1 wherein the polypeptide having fructokinase activity has at least 95% sequence identity, based on a Clustal V method of alignment, when compared to an amino acid sequence as set forth in SEQ ID NO:44, SEQ ID NO:46, SEQ ID NO:48, SEQ ID NO:50, SEQ ID NO:52, SEQ ID NO:54, SEQ ID NO:85, or SEQ ID NO:87.

6. The recombinant bacterium of claim 1 wherein the polypeptide having sucrose hydrolase activity has at least 95% sequence identity, based on a Clustal V method of alignment, when compared to an amino acid sequence as set forth in SEQ ID NO:56, SEQ ID NO:58, SEQ ID NO:60, SEQ ID NO:62, SEQ ID NO:64, SEQ ID NO:66, or SEQ ID NO:68.

7. The recombinant bacterium of claim 1 wherein the polypeptide having sucrose transporter activity corresponds substantially to the sequence set forth in SEQ ID NO:26.

8. The recombinant bacterium of claim 1 wherein the polypeptide having fructokinase activity corresponds substantially to the sequence set forth in SEQ ID NO:48.

9. The recombinant bacterium of claim 1 wherein the polypeptide having sucrose hydrolase activity corresponds substantially to the sequence set forth in SEQ ID NO:58.

10. The recombinant bacterium of any of claims 1-9 wherein said bacterium is selected from the group consisting of the genera: Escherichia, Klebsiella, Citrobacter, and Aerobacter.

11. The recombinant bacterium of claim 10 wherein said bacterium is Escherichia coli.

12. A process for making glycerol, 1,3-propanediol and/or 3-hydroxypropionic acid from sucrose comprising:
a) culturing the recombinant bacterium of any of claims 1-9 in the presence of sucrose; and
b) optionally, recovering the glycerol, 1,3-propanediol and/or 3-hydroxypropionic acid produced.

13. A process for making glycerol, 1,3-propanediol and/or 3-hydroxypropionic acid from sucrose comprising:
a) culturing the recombinant bacterium of claim 10 in the presence of sucrose; and
b) optionally, recovering the glycerol, 1,3-propanediol and/or 3-hydroxypropionic acid produced.
